SEQUENCE LISTING (69 sequences)

SEQ ID NO: 1 - DNA, 16 bp, Homo sapiens
  source 1..16: mol_type = other DNA
tcgtgaggattggtcg

SEQ ID NO: 2 - DNA, 16 bp, Homo sapiens
  source 1..16: mol_type = other DNA
ccgtgcactttggtca

SEQ ID NO: 3 - DNA, 16 bp, Homo sapiens
  source 1..16: mol_type = other DNA
caatgccgtggaagtc

SEQ ID NO: 4 - DNA, 16 bp, Homo sapiens
  source 1..16: mol_type = other DNA
tgaggattggtcggtg

SEQ ID NO: 5 - DNA, 16 bp, Homo sapiens
  source 1..16: mol_type = other DNA
cgtggaagtcaaattt

SEQ ID NO: 6 - DNA, 16 bp, Homo sapiens
  source 1..16: mol_type = other DNA
gatagtgcaagaagta

SEQ ID NO: 7 - DNA, 16 bp, Homo sapiens
  source 1..16: mol_type = other DNA
gggcggtaaataagaa

SEQ ID NO: 8 - DNA, 16 bp, Homo sapiens
  source 1..16: mol_type = other DNA
cgtgcactttggtcat

SEQ ID NO: 9 - DNA, 16 bp, Homo sapiens
  source 1..16: mol_type = other DNA
gcgattgattcgatag

SEQ ID NO: 10 - DNA, 16 bp, Homo sapiens
  source 1..16: mol_type = other DNA
aacagcgattgattcg

SEQ ID NO: 11 - DNA, 16 bp, Mus musculus
  source 1..16: mol_type = other DNA
aaagtcacgattacct

SEQ ID NO: 12 - DNA, 16 bp, Mus musculus
  source 1..16: mol_type = other DNA
ccgtatgctgaatcaa

SEQ ID NO: 13 - DNA, 16 bp, Mus musculus
  source 1..16: mol_type = other DNA
atttaactcattcgat

SEQ ID NO: 14 - DNA, 16 bp, synthetic construct
  misc_feature 1..3: note = RNA
  misc_feature 14..16: note = RNA
  misc_feature 3..13: note = DNA
  source 1..16: mol_type = other DNA
  modified_base 1..2: mod_base = cm
  modified_base 3: mod_base = gm
  modified_base 14: mod_base = um
  modified_base 15: mod_base = cm
  modified_base 16: mod_base = OTHER; note = 2-O-methyladenosine
ccgtgcactttggtca

SEQ ID NO: 15 - DNA, 16 bp, synthetic construct
  misc_feature 1..3: note = RNA
  misc_feature 13..16: note = RNA
  misc_feature 3..12: note = DNA
  source 1..16: mol_type = other DNA
  modified_base 1..2: mod_base = cm
  modified_base 3: mod_base = gm
  modified_base 13: mod_base = gm
  modified_base 14: mod_base = um
  modified_base 15: mod_base = cm
  modified_base 16: mod_base = OTHER; note = 2-O-methyladenosine
ccgtgcactttggtca

SEQ ID NO: 16 - DNA, 16 bp, synthetic construct
  source 1..16: mol_type = other DNA
  misc_feature 1..3: note = RNA
  misc_feature 4..14: note = DNA
  misc_feature 15..16: note = RNA
  modified_base 1..2: mod_base = cm
  modified_base 3: mod_base = gm
  modified_base 15: mod_base = cm
  modified_base 16: mod_base = OTHER; note = 2-O-methyladenosine
ccgtgcactttggtca

SEQ ID NO: 17 - DNA, 16 bp, synthetic construct
  source 1..16: mol_type = other DNA
  misc_feature 1..4: note = RNA
  misc_feature 5..13: note = DNA
  misc_feature 14..16: note = RNA
  modified_base 1..2: mod_base = cm
  modified_base 3: mod_base = gm
  modified_base 4: mod_base = um
  modified_base 14: mod_base = um
  modified_base 15: mod_base = cm
  modified_base 16: mod_base = OTHER; note = 2-O-methyladenosine
ccgtgcactttggtca

SEQ ID NO: 18 - DNA, 16 bp, synthetic construct
  misc_feature 1..4: note = RNA
  misc_feature 5..12: note = DNA
  misc_feature 13..16: note = RNA
  source 1..16: mol_type = other DNA
  modified_base 1..2: mod_base = cm
  modified_base 3: mod_base = gm
  modified_base 4: mod_base = um
  modified_base 13: mod_base = gm
  modified_base 14: mod_base = um
  modified_base 15: mod_base = cm
  modified_base 16: mod_base = OTHER; note = 2-O-methyladenosine
ccgtgcactttggtca

SEQ ID NO: 19 - DNA, 16 bp, synthetic construct
  misc_feature 5..14: note = DNA
  misc_feature 1..4: note = RNA
  misc_feature 15..16: note = RNA
  source 1..16: mol_type = other DNA
  modified_base 1..2: mod_base = cm
  modified_base 3: mod_base = gm
  modified_base 4: mod_base = um
  modified_base 15: mod_base = cm
  modified_base 16: mod_base = OTHER; note = 2-O-methyladenosine
ccgtgcactttggtca

SEQ ID NO: 20 - DNA, 16 bp, synthetic construct
  misc_feature 1..2: note = RNA
  misc_feature 14..16: note = RNA
  misc_feature 3..13: note = DNA
  source 1..16: mol_type = other DNA
  modified_base 1..2: mod_base = cm
  modified_base 14: mod_base = um
  modified_base 15: mod_base = cm
  modified_base 16: mod_base = OTHER; note = 2-O-methyladenosine
ccgtgcactttggtca

SEQ ID NO: 21 - DNA, 16 bp, synthetic construct
  misc_feature 1..2: note = RNA
  misc_feature 13..16: note = RNA
  misc_feature 3..12: note = DNA
  source 1..16: mol_type = other DNA
  modified_base 1..2: mod_base = cm
  modified_base 13: mod_base = gm
  modified_base 14: mod_base = um
  modified_base 15: mod_base = cm
  modified_base 16: mod_base = OTHER; note = 2-O-methyladenosine
ccgtgcactttggtca

SEQ ID NO: 22 - DNA, 16 bp, synthetic construct
  source 1..16: mol_type = other DNA
  misc_feature 1..2: note = RNA
  misc_feature 3..14: note = DNA
  misc_feature 15..16: note = RNA
  modified_base 1..2: mod_base = cm
  modified_base 15: mod_base = cm
  modified_base 16: mod_base = OTHER; note = 2-O-methyladenosine
ccgtgcactttggtca

SEQ ID NO: 23 - DNA, 16 bp, synthetic construct
  source 1..16: mol_type = other DNA
  modified_base 1..3: mod_base = OTHER; note = LNA
  modified_base 14..16: mod_base = OTHER; note = LNA
ccgtgcactttggtca

SEQ ID NO: 24 - DNA, 16 bp, synthetic construct
  source 1..16: mol_type = other DNA
  modified_base 1..3: mod_base = OTHER; note = LNA
  modified_base 13..16: mod_base = OTHER; note = LNA
ccgtgcactttggtca

SEQ ID NO: 25 - DNA, 16 bp, synthetic construct
  source 1..16: mol_type = other DNA
  modified_base 1..3: mod_base = OTHER; note = LNA
  modified_base 15..16: mod_base = OTHER; note = LNA
ccgtgcactttggtca

SEQ ID NO: 26 - DNA, 16 bp, synthetic construct
  source 1..16: mol_type = other DNA
  modified_base 1..4: mod_base = OTHER; note = LNA
  modified_base 14..16: mod_base = OTHER; note = LNA
ccgtgcactttggtca

SEQ ID NO: 27 - DNA, 16 bp, synthetic construct
  source 1..16: mol_type = other DNA
  modified_base 1..4: mod_base = OTHER; note = LNA
  modified_base 13..16: mod_base = OTHER; note = LNA
ccgtgcactttggtca

SEQ ID NO: 28 - DNA, 16 bp, synthetic construct
  source 1..16: mol_type = other DNA
  modified_base 1..4: mod_base = OTHER; note = LNA
  modified_base 15..16: mod_base = OTHER; note = LNA
ccgtgcactttggtca

SEQ ID NO: 29 - DNA, 16 bp, synthetic construct
  source 1..16: mol_type = other DNA
  modified_base 1..2: mod_base = OTHER; note = LNA
  modified_base 15..16: mod_base = OTHER; note = LNA
ccgtgcactttggtca

SEQ ID NO: 30 - DNA, 16 bp, synthetic construct
  source 1..16: mol_type = other DNA
  modified_base 1..2: mod_base = OTHER; note = LNA
  modified_base 14..16: mod_base = OTHER; note = LNA
ccgtgcactttggtca

SEQ ID NO: 31 - DNA, 16 bp, synthetic construct
  source 1..16: mol_type = other DNA
  modified_base 1..2: mod_base = OTHER; note = LNA
  modified_base 13..16: mod_base = OTHER; note = LNA
ccgtgcactttggtca

SEQ ID NO: 32 - DNA, 16 bp, synthetic construct
  misc_feature 1..3: note = RNA
  misc_feature 4..13: note = DNA
  misc_feature 14..16: note = RNA
  source 1..16: mol_type = other DNA
  modified_base 1: mod_base = gm
  modified_base 2: mod_base = cm
  modified_base 3: mod_base = gm
  modified_base 14: mod_base = um
  modified_base 15: mod_base = OTHER; note = 2-O-methyladenosine
  modified_base 16: mod_base = gm
gcgattgattcgatag

SEQ ID NO: 33 - DNA, 16 bp, synthetic construct
  misc_feature 1..3: note = RNA
  misc_feature 4..12: note = DNA
  misc_feature 13..16: note = RNA
  source 1..16: mol_type = other DNA
  modified_base 1: mod_base = gm
  modified_base 2: mod_base = cm
  modified_base 3: mod_base = gm
  modified_base 13: mod_base = OTHER; note = 2-O-methyladenosine
  modified_base 14: mod_base = um
  modified_base 15: mod_base = OTHER; note = 2-O-methyladenosine
  modified_base 16: mod_base = gm
gcgattgattcgatag

SEQ ID NO: 34 - DNA, 16 bp, synthetic construct
  misc_feature 1..3: note = RNA
  misc_feature 4..14: note = DNA
  misc_feature 15..16: note = RNA
  source 1..16: mol_type = other DNA
  modified_base 1: mod_base = gm
  modified_base 2: mod_base = cm
  modified_base 3: mod_base = gm
  modified_base 15: mod_base = OTHER; note = 2-O-methyladenosine
  modified_base 16: mod_base = gm
gcgattgattcgatag

SEQ ID NO: 35 - DNA, 16 bp, synthetic construct
  misc_feature 1..4: note = RNA
  misc_feature 5..13: note = DNA
  misc_feature 14..16: note = RNA
  source 1..16: mol_type = other DNA
  modified_base 1: mod_base = gm
  modified_base 2: mod_base = cm
  modified_base 14: mod_base = um
  modified_base 3: mod_base = gm
  modified_base 4: mod_base = OTHER; note = 2-O-Methyladenosine
  modified_base 15: mod_base = OTHER; note = 2-O-Methyladenosine
  modified_base 16: mod_base = gm
gcgattgattcgatag

SEQ ID NO: 36 - DNA, 16 bp, synthetic construct
  misc_feature 1..4: note = RNA
  misc_feature 5..12: note = DNA
  misc_feature 13..16: note = RNA
  source 1..16: mol_type = other DNA
  modified_base 1: mod_base = gm
  modified_base 2: mod_base = cm
  modified_base 3: mod_base = gm
  modified_base 4: mod_base = OTHER; note = 2-O-Methyladenosine
  modified_base 13: mod_base = OTHER; note = 2-O-Methyladenosine
  modified_base 14: mod_base = um
  modified_base 15: mod_base = OTHER; note = 2-O-Methyladenosine
  modified_base 16: mod_base = gm
gcgattgattcgatag

SEQ ID NO: 37 - DNA, 16 bp, synthetic construct
  misc_feature 1..4: note = RNA
  misc_feature 5..14: note = DNA
  misc_feature 15..16: note = RNA
  source 1..16: mol_type = other DNA
  modified_base 1: mod_base = gm
  modified_base 2: mod_base = cm
  modified_base 3: mod_base = gm
  modified_base 4: mod_base = OTHER; note = 2-O-Methyladenosine
  modified_base 15: mod_base = OTHER; note = 2-O-Methyladenosine
  modified_base 16: mod_base = gm
gcgattgattcgatag

SEQ ID NO: 38 - DNA, 16 bp, synthetic construct
  misc_feature 1..2: note = RNA
  misc_feature 3..13: note = DNA
  misc_feature 14..16: note = RNA
  source 1..16: mol_type = other DNA
  modified_base 1: mod_base = gm
  modified_base 2: mod_base = cm
  modified_base 14: mod_base = um
  modified_base 15: mod_base = OTHER; note = 2-O-Methyladenosine
  modified_base 16: mod_base = gm
gcgattgattcgatag

SEQ ID NO: 39 - DNA, 16 bp, synthetic construct
  misc_feature 1..2: note = RNA
  misc_feature 3..12: note = DNA
  misc_feature 13..16: note = RNA
  source 1..16: mol_type = other DNA
  modified_base 1: mod_base = gm
  modified_base 2: mod_base = cm
  modified_base 13: mod_base = OTHER; note = 2-O-Methyladenosine
  modified_base 14: mod_base = um
  modified_base 15: mod_base = OTHER; note = 2-O-Methyladenosine
  modified_base 16: mod_base = gm
gcgattgattcgatag

SEQ ID NO: 40 - DNA, 16 bp, synthetic construct
  misc_feature 1..2: note = RNA
  misc_feature 3..14: note = DNA
  misc_feature 15..16: note = RNA
  source 1..16: mol_type = other DNA
  modified_base 1: mod_base = gm
  modified_base 2: mod_base = cm
  modified_base 15: mod_base = OTHER; note = 2-O-Methyladenosine
  modified_base 16: mod_base = gm
gcgattgattcgatag

SEQ ID NO: 41 - DNA, 16 bp, synthetic construct
  source 1..16: mol_type = other DNA
  modified_base 1..3: mod_base = OTHER; note = LNA
  modified_base 14..16: mod_base = OTHER; note = LNA
gcgattgattcgatag

SEQ ID NO: 42 - DNA, 16 bp, synthetic construct
  source 1..16: mol_type = other DNA
  modified_base 1..3: mod_base = OTHER; note = LNA
  modified_base 13..16: mod_base = OTHER; note = LNA
gcgattgattcgatag

SEQ ID NO: 43 - DNA, 16 bp, synthetic construct
  source 1..16: mol_type = other DNA
  modified_base 1..3: mod_base = OTHER; note = LNA
  modified_base 15..16: mod_base = OTHER; note = LNA
gcgattgattcgatag

SEQ ID NO: 44 - DNA, 16 bp, synthetic construct
  source 1..16: mol_type = other DNA
  modified_base 1..4: mod_base = OTHER; note = LNA
  modified_base 14..16: mod_base = OTHER; note = LNA
gcgattgattcgatag

SEQ ID NO: 45 - DNA, 16 bp, synthetic construct
  source 1..16: mol_type = other DNA
  modified_base 1..4: mod_base = OTHER; note = LNA
  modified_base 13..16: mod_base = OTHER; note = LNA
gcgattgattcgatag

SEQ ID NO: 46 - DNA, 16 bp, synthetic construct
  source 1..16: mol_type = other DNA
  modified_base 1..4: mod_base = OTHER; note = LNA
  modified_base 15..16: mod_base = OTHER; note = LNA
gcgattgattcgatag

SEQ ID NO: 47 - DNA, 16 bp, synthetic construct
  source 1..16: mol_type = other DNA
  modified_base 1..2: mod_base = OTHER; note = LNA
  modified_base 14..16: mod_base = OTHER; note = LNA
gcgattgattcgatag

SEQ ID NO: 48 - DNA, 16 bp, synthetic construct
  source 1..16: mol_type = other DNA
  modified_base 1..2: mod_base = OTHER; note = LNA
  modified_base 13..16: mod_base = OTHER; note = LNA
gcgattgattcgatag

SEQ ID NO: 49 - DNA, 16 bp, synthetic construct
  source 1..16: mol_type = other DNA
  modified_base 1..2: mod_base = OTHER; note = LNA
  modified_base 15..16: mod_base = OTHER; note = LNA
gcgattgattcgatag

SEQ ID NO: 50 - DNA, 16 bp, synthetic construct
  misc_feature 1..3: note = RNA
  misc_feature 4..13: note = DNA
  misc_feature 14..16: note = RNA
  source 1..16: mol_type = other DNA
  modified_base 1..2: mod_base = OTHER; note = 2-O-Methyladenosine
  modified_base 3: mod_base = cm
  modified_base 14: mod_base = um
  modified_base 15: mod_base = cm
  modified_base 16: mod_base = gm
aacagcgattgattcg

SEQ ID NO: 51 - DNA, 16 bp, synthetic construct
  misc_feature 1..3: note = RNA
  misc_feature 4..12: note = DNA
  misc_feature 13..16: note = RNA
  source 1..16: mol_type = other DNA
  modified_base 1..2: mod_base = OTHER; note = 2-O-Methyladenosine
  modified_base 3: mod_base = cm
  modified_base 13..14: mod_base = OTHER; note = 2-O-Methyladenosine
  modified_base 15: mod_base = cm
  modified_base 16: mod_base = gm
aacagcgattgattcg

SEQ ID NO: 52 - DNA, 16 bp, synthetic construct
  misc_feature 1..3: note = RNA
  misc_feature 4..14: note = DNA
  misc_feature 15..16: note = RNA
  source 1..16: mol_type = other DNA
  modified_base 1..2: mod_base = OTHER; note = 2-O-Methyladenosine
  modified_base 3: mod_base = cm
  modified_base 15: mod_base = cm
  modified_base 16: mod_base = gm
aacagcgattgattcg

SEQ ID NO: 53 - DNA, 16 bp, synthetic construct
  misc_feature 1..4: note = RNA
  misc_feature 5..13: note = DNA
  misc_feature 14..16: note = RNA
  source 1..16: mol_type = other DNA
  modified_base 1..2: mod_base = OTHER; note = 2-O-Methyladenosine
  modified_base 3: mod_base = cm
  modified_base 4: mod_base = OTHER; note = 2-O-Methyladenosine
  modified_base 14: mod_base = um
  modified_base 15: mod_base = cm
  modified_base 16: mod_base = gm
aacagcgattgattcg

SEQ ID NO: 54 - DNA, 16 bp, synthetic construct
  misc_feature 1..4: note = RNA
  misc_feature 5..12: note = DNA
  misc_feature 13..16: note = RNA
  source 1..16: mol_type = other DNA
  modified_base 1..2: mod_base = OTHER; note = 2-O-Methyladenosine
  modified_base 3: mod_base = cm
  modified_base 4: mod_base = OTHER; note = 2-O-Methyladenosine
  modified_base 13..14: mod_base = OTHER; note = 2-O-Methyladenosine
  modified_base 15: mod_base = cm
  modified_base 16: mod_base = gm
aacagcgattgattcg

SEQ ID NO: 55 - DNA, 16 bp, synthetic construct
  misc_feature 1..4: note = RNA
  misc_feature 5..14: note = DNA
  misc_feature 15..16: note = RNA
  source 1..16: mol_type = other DNA
  modified_base 1..2: mod_base = OTHER; note = 2-O-Methyladenosine
  modified_base 3: mod_base = cm
  modified_base 4: mod_base = OTHER; note = 2-O-Methyladenosine
  modified_base 15: mod_base = cm
  modified_base 16: mod_base = gm
aacagcgattgattcg

SEQ ID NO: 56 - DNA, 16 bp, synthetic construct
  misc_feature 1..2: note = RNA
  misc_feature 3..13: note = DNA
  misc_feature 14..16: note = RNA
  source 1..16: mol_type = other DNA
  modified_base 1..2: mod_base = OTHER; note = 2-O-Methyladenosine
  modified_base 14: mod_base = um
  modified_base 15: mod_base = cm
  modified_base 16: mod_base = gm
aacagcgattgattcg

SEQ ID NO: 57 - DNA, 16 bp, synthetic construct
  misc_feature 1..2: note = RNA
  misc_feature 3..12: note = DNA
  misc_feature 13..16: note = RNA
  source 1..16: mol_type = other DNA
  modified_base 1..2: mod_base = OTHER; note = 2-O-Methyladenosine
  modified_base 13..14: mod_base = um
  modified_base 15: mod_base = cm
  modified_base 16: mod_base = gm
aacagcgattgattcg

SEQ ID NO: 58 - DNA, 16 bp, synthetic construct
  misc_feature 1..2: note = RNA
  misc_feature 3..14: note = DNA
  misc_feature 15..16: note = RNA
  source 1..16: mol_type = other DNA
  modified_base 1..2: mod_base = OTHER; note = 2-O-Methyladenosine
  modified_base 15: mod_base = cm
  modified_base 16: mod_base = gm
aacagcgattgattcg

SEQ ID NO: 59 - DNA, 16 bp, synthetic construct
  source 1..16: mol_type = other DNA
  modified_base 1..3: mod_base = OTHER; note = LNA
  modified_base 14..16: mod_base = OTHER; note = LNA
aacagcgattgattcg

SEQ ID NO: 60 - DNA, 16 bp, synthetic construct
  source 1..16: mol_type = other DNA
  modified_base 1..3: mod_base = OTHER; note = LNA
  modified_base 13..16: mod_base = OTHER; note = LNA
aacagcgattgattcg

SEQ ID NO: 61 - DNA, 16 bp, synthetic construct
  source 1..16: mol_type = other DNA
  modified_base 1..3: mod_base = OTHER; note = LNA
  modified_base 15..16: mod_base = OTHER; note = LNA
aacagcgattgattcg

SEQ ID NO: 62 - DNA, 16 bp, synthetic construct
  source 1..16: mol_type = other DNA
  modified_base 1..4: mod_base = OTHER; note = LNA
  modified_base 15..16: mod_base = OTHER; note = LNA
aacagcgattgattcg

SEQ ID NO: 63 - DNA, 16 bp, synthetic construct
  source 1..16: mol_type = other DNA
  modified_base 1..4: mod_base = OTHER; note = LNA
  modified_base 13..16: mod_base = OTHER; note = LNA
aacagcgattgattcg

SEQ ID NO: 64 - DNA, 16 bp, synthetic construct
  source 1..16: mol_type = other DNA
  modified_base 1..4: mod_base = OTHER; note = LNA
  modified_base 15..16: mod_base = OTHER; note = LNA
aacagcgattgattcg

SEQ ID NO: 65 - DNA, 16 bp, synthetic construct
  source 1..16: mol_type = other DNA
  modified_base 1..2: mod_base = OTHER; note = LNA
  modified_base 14..16: mod_base = OTHER; note = LNA
aacagcgattgattcg

SEQ ID NO: 66 - DNA, 16 bp, synthetic construct
  source 1..16: mol_type = other DNA
  modified_base 1..2: mod_base = OTHER; note = LNA
  modified_base 13..16: mod_base = OTHER; note = LNA
aacagcgattgattcg

SEQ ID NO: 67 - DNA, 16 bp, synthetic construct
  source 1..16: mol_type = other DNA
  modified_base 1..2: mod_base = OTHER; note = LNA
  modified_base 15..16: mod_base = OTHER; note = LNA
aacagcgattgattcg

SEQ ID NO: 68 - RNA, 16 nt, synthetic construct
  source 1..16: mol_type = other RNA
  modified_base 1: mod_base = um
  modified_base 2: mod_base = gm
  modified_base 3: mod_base = OTHER; note = 2'-O-Methyladenosine
  modified_base 14: mod_base = cm
  modified_base 15..16: mod_base = gm
tgaccaaagtgcacgg

SEQ ID NO: 69 - DNA, 32 bp, synthetic construct
  source 1..32: mol_type = other DNA
  misc_feature 1..16: note = DNA
  misc_feature 17..32: note = RNA
  misc_recomb 16^17: note = duplex
  modified_base 1..3: mod_base = OTHER; note = LNA
  modified_base 14..16: mod_base = OTHER; note = LNA
  modified_base 17: mod_base = um
  modified_base 18: mod_base = gm
  modified_base 19: mod_base = OTHER; note = 2-O-Methyladenosine
  modified_base 30: mod_base = cm
  modified_base 31..32: mod_base = gm
ccgtgcactttggtcatgaccaaagtgcacgg

(19) United States
(12) Patent Application Publication
(10) Pub. No.: US 2025/0283075 A1
(43) Pub. Date: Sep. 11, 2025

(54) COMPOSITIONS FOR TARGETING FLI-1 AND METHODS OF USE THEREOF

(71) Applicant: MUSC Foundation for Research Development d/b/a Zucker Institute for Innovation Commercialization, Charleston, SC (US)

(72) Inventors: Hongkuan FAN, Mount Pleasant, SC (US); Andrew GOODWIN, Mount Pleasant, SC (US); Perry HALUSHKA, Charleston, SC (US); Pengfei LI, Charleston, SC (US); Yan WU, Charleston, SC (US)

(73) Assignee: MUSC FOUNDATION FOR RESEARCH DEVELOPMENT, Charleston, SC (US)

(21) Appl. No.: 18/685,858

(22) PCT Filed: Aug. 26, 2022
(86) PCT No.: PCT/US2022/075544

§ 371 Date: Feb. 22, 2024

Related U.S. Application Data
(60) Provisional application No. 63/237,746, filed on Aug. 27, 2021.

(51) Int. Cl. C12N 15/113; A61P 25/28

(52) U.S. Cl. CPC C12N 15/113; A61P 25/28; C12N 2310/11; C12N 2310/315; C12N 2310/341

(57) ABSTRACT

The present disclosure provides methods for treating inflammatory disease, such as Alzheimer's disease, by administering an inhibitor of Fli-1, such as an antisense oligonucleotide. Further provided are antisense gapmer oligonucleotides for targeting Fli-1.

### PRIORITY CLAIM

This application claims benefit of priority to U.S. Provisional Application Ser. No. 63/237,746 filed Aug. 27, 2021, the entire contents of which are hereby incorporated by reference.

The invention was made with government support under Grant No. NIH R01 GM130653 awarded by the National Institute of Health (NIH). The government has certain rights in the invention.

### INCORPORATION OF A SEQUENCE LISTING

This application contains a Sequence Listing XML, which has been submitted electronically and is hereby incorporated by reference in its entirety. Said XML Sequence Listing, created on Aug. 25, 2022, is named MESCP0131WO.xml and is 183,337 bytes in size.

### BACKGROUND

### 1. Field

The disclosure relates generally to the field of molecular biology. More particularly, it concerns methods of compositions for targeting FLI-1 and methods of use thereof.

### 2. Related Art

Alzheimer's disease (AD) is a complex disease associated with cognitive impairment, accumulation of amyloid β-peptide (Aβ), vascular dysfunction and neuroinflammation (Jack et al., 2018) (Medrano-Jimenez et al., 2019) (Sweeney et al., 2018). Cerebral vascular dysfunction such as decreases in cerebral blood flow (CBF) and disruption of the blood brain barrier (BBB) has been recognized as an early and pivotal contributor to AD pathogenesis, and a reliable predictor of cognitive decline (Kisler et al., 2017) (Iadecola et al., 2019). BBB damage in patients with AD and animal models is associated with degeneration of brain pericytes, a vascular mural cell that regulates CBF, maintains BBB integrity, mediates neuroinflammation, and exhibits phagocytic activity to remove toxic endogenous proteins, including Aβ (Halliday et al., 2016) (Ma et al., 2018) (Tachibana et al., 2018) (Brown et al, 2019) (Wu et al., 2019). A deficiency of brain pericytes in the murine central nervous system leads to BBB breakdown (Sengillo et al., 2013) (Armulik et al., 2010). Furthermore, reduced pericyte number was observed and correlated with BBB breakdown and Aβ deposition in the hippocampus and retina from patients with AD and animal models (Wu et al., 2019) (Sengillo et al., 2013) (Shi et al., 2020a) (Shi et al., 2020b). Moreover, pericyte deficiency in APP/PS1 mice was reported to accelerate BBB breakdown and increase Aβ accumulation in the brain (Sagare et al., 2013). However, the processes that govern pericyte viability and their role in AD development have not been fully elucidated.

Previous studies have demonstrated that the friend leukemia virus integration 1 (Fli-1), an ETS transcription factor, governs lung pericyte dysfunction and viability via the mediation of pericyte pyroptosis (Li et al., 2018). Specifically, Fli-1 binds to the promoter regions of caspase ⅓ and regulates caspase-⅓ expression (Sohn et al., 2010) (Li et al., 2019). In addition, Fli-1 is involved in a wide spectrum of biological processes, including cancer development, fibrosis, vasculopathy and inflammation (Takahashi et al., 2017) (Akamata et al., 2015) (Theisen et al., 2016) (Sato et al., 2014), and it regulates the expression of important cytokines and matrix metalloproteases (MMP) such as IL-6 and MMP3, which are implicated in AD (Sato et al., 2014) (Cai et al., 2014) (Pillai et al., 2019) (Ho & Ivashkiv, 2010). However, the role of Fli-1 and its impact on pericyte degeneration in AD has not been previously investigated.

### SUMMARY

In a first embodiment, the present disclosure provides methods for preventing and/or treating an inflammatory disease in a subject comprising administering an effective amount of friend leukemia virus integration 1 (Fli-1) inhibitor to the subject.

In some aspects, the Fli-1 inhibitor is a small molecule, such as camptothecin (CPT), topotecan (TPT), A661, or A665.

In certain aspects, the Fli-1 inhibitor comprises an oligonucleotide. In some aspects, the oligonucleotide is double-stranded. In particular aspects, the oligonucleotide is small interfering RNA (siRNA) or short hairpin RNA (shRNA).

In some aspects, the oligonucleotide comprises at least one modified nucleotide. For example, the at least one modification is selected from the group consisting of 2′-O-methyl-ribonucleotides, 2′-fluoro-ribonucleotides, 5′-vinylphosphonate, phosphorothioate linkage, or lipid conjugation.

In some aspects, the oligonucleotide is a single-stranded oligonucleotide, such as a single-stranded antisense oligonucleotide. In specific aspects, the single-stranded antisense oligonucleotide has a length of 14-22 nucleotides, such as 14-17 nucleosides, particularly 16 nucleosides.

In certain aspects, the oligonucleotide comprises one or more chemically-modified nucleobases. In some aspects, the one or more chemically-modified nucleobases is a nuclease-resistant modification. In certain aspects, the nuclease-resistant modification is a modified sugar moiety or a modified internucleoside linkage. In particular aspects, modified sugar moiety is a high-affinity sugar modification. In some aspects, the oligonucleotide comprises one or more locked nucleic acids (LNAs). In specific aspects, the oligonucleotide comprises DNA and/or RNA nucleobases. In certain aspects, the oligonucleotide comprises DNA and RNA nucleobases. In some aspects, the oligonucleotide comprises a phosphorothioate (PS) backbone modification.

In particular aspects, the single-stranded antisense oligonucleotide is gapmer. In specific aspects, the gapmer is a LNA gapmer. In some aspects, the gapmer comprises a 5′-flank, a gap and a 3′-flank. In particular aspects, the gap comprises DNA nucleobases. In specific aspects, the gap comprises 8-12 DNA nucleobases, such as 10 DNA nucleobases. In some aspects, the 5′-flank and/or 3′-flank comprise RNA nucleobases. In particular aspects, the 5′-flank and/or 3′-flank comprise 2-4 RNA nucleobases, such as 3 RNA nucleobases. In specific aspects, the 5′-flank and/or 3′-flank comprise or consist of LNA nucleosides. In some aspects, the gapmer comprises a sequence having 80% sequence identity to SEQ ID NO:1, 2, 3, 4, 5, 6, 7, 8, 9, or 10. In particular aspects, the gapmer comprises a sequence having 80% (e.g., 85%, 90%, 91%, 92%, 93%, 94%, 95%, 96%, 97%, 98%, or 99%) sequence identity to SEQ ID NO:2, 9, or 10. In some aspects, the gapmer comprises a sequence having 80% sequence identity to SEQ ID NO:2. In certain aspects, the gapmer comprises SEQ ID NO: 1, 2, 3, 4, 5, 6, 7, 8, 9, or 10. In certain aspects, the gapmer comprises SEQ ID NO: 2, 9, or 10. In particular aspects, the gapmer comprises SEQ ID NO:2. In specific aspects, the gapmer comprises SEQ ID NO: 14, 23, 24, 25, 41, 48, 59, or 64.

In some aspects, the gapmer comprises at least one 2′ sugar modification. In certain aspects, the gapmer comprises at least one 2′-O-methyl (2′-O-Me), 2′-O-methoxyethyl (2′-O-MOE), or 2′-fluoro (2′-F) modification.

In certain aspects, the gapmer is hydridized to a complementary RNA to form a DNA:RNA heteroduplex. In some aspects, the RNA comprises a sequence of SEQ ID NO: 69. In certain aspects, the DNA:RNA heteroduplex is covalently bound to a lipid. In some aspects, the lipid is cholesterol, a tocopherol, a tocotrienol, a fatty acid, a lipid-soluble vitamin, a glycolipid, or a glyceride. In some aspects, the DNA:RNA heteroduplex is covalently bound to cholesterol. In some aspects, the cholesterol comprises a triethylene glycol spacer (TEG). In certain aspects, the DNA:RNA heteroduplex bound to cholesterol has a sequence of SEQ ID NO: 70.

In some aspects, the Fli-1 inhibitor is a Fli-1 antibody. In certain aspects, the antibody is recombinant. In some aspects, the antibody is an IgG, IgM, IgA or an antigen binding fragment thereof. In certain aspects, the antibody is a Fab′, a F(ab′)2, a F(ab′)3, a monovalent scFv, a bivalent scFv, or a single domain antibody. In some aspects, the antibody is a human, humanized antibody or de-immunized antibody. In some aspects, the antibody is conjugated to an imaging agent, a chemotherapeutic agent, a toxin or a radionuclide. In certain aspects, the Fli-1 antibody is encapsulated in a nanoparticle, liposome, polymeric nanocarrier, polymeric micelle, or nanogel. In some aspects, the Fli-1 antibody is encapsulated in a polymer nanogel. In some aspects, the polymer nanogel is a Poly[(2-(pyridin-2-yldisulfanyl)ethyl acrylate)-co-[poly(ethylene glycol)]] (PDA-PEG) polymer nanogel.

In certain aspects, the inflammatory disease is Alzheimer's disease. In some aspects, the inflammatory disease is systemic lupus erythematosus, sepsis, or multiple sclerosis.

In some aspects, administering the Fli-1 inhibitor results in decreased pericyte loss, improvement in cognitive deficits, reduced Aβ deposition, and/or decreased BBB breakdown. In some aspects, administering the Fli-1 inhibitor results in decreased BACE1, ICAM1 and/or VCAM1 levels in the hippocampus of the subject as compared to expression prior to administering. In certain aspects, administering the Fli-1 inhibitor results in decreased expression of Fli-1 in the brain of the subject as compared to Fli-1 expression prior to administering. In some aspects, administering the Fli-1 inhibitor results in improved spatial learning and/or memory impairment.

In some aspects, the Fli-1 inhibitor is administered more than once. In certain aspects, the Fli-1 inhibitor is administered at least once every week. In some aspects, the Fli-1 inhibitor is administered systemically. In some aspects, the Fli-1 inhibitor is administered topically, orally, intravenously, intraarterially, intrathecally, intranasally or intramuscularly. In certain aspects, the Fli-1 inhibitor is administered to the brain. In particular aspects, the Fli-1 inhibitor is administered by intrahippocampal injection or intravenous injection. In some aspects, the Fli-1 inhibitor is administered by a liposomal, exosomal or nanoparticle formulation. In certain aspects, the Fli-1 inhibitor is administered by extracellular vesicles (EVs). In some aspects, the EVs are derived from mesenchymal stem cells (MSC) s or endothelial progenitor cells (EPCs). In certain aspects, the EVs are administered intranasally. In certain aspects, the nanoparticle formulation comprises brain-targeted nanoparticles, such as brain-targeted nanoparticles capable of crossing the blood brain barrier. In some aspects, the nanoparticles are dual-targeted and dual-responsive nanoparticles (DTDRN). In certain aspects, the brain-targeted nanoparticles are conjugated to scopine and/or glutathione.

In further aspects, the method further comprises administering a second therapeutic agent to the subject.

A further embodiment provides a composition comprising an antisense gapmer oligonucleotide, wherein the gapmer oligonucleotide is 11-25 nucleotide units in length and able to recruit RNaseH when hybridized to Fli-1.

In specific aspects, the gapmer is a LNA gapmer. In some aspects, the gapmer comprises a 5′-flank, a gap and a 3′-flank. In particular aspects, the gap comprises DNA nucleobases. In specific aspects, the gap comprises 8-12 DNA nucleobases, such as 10 DNA nucleobases. In some aspects, the 5′-flank and/or 3′-flank comprise RNA nucleobases. In particular aspects, the 5′-flank and/or 3′-flank comprise 2-4 RNA nucleobases, such as 3 RNA nucleobases. In specific aspects, the 5′-flank and/or 3′-flank comprise or consist of LNA nucleosides. In some aspects, the gapmer comprises a sequence having 80% sequence identity to SEQ ID NO:1, 2, 3, 4, 5, 6, 7, 8, 9, or 10. In particular aspects, the gapmer comprises a sequence having 80% (e.g., 85%, 90%, 91%, 92%, 93%, 94%, 95%, 96%, 97%, 98%, or 99%) sequence identity to SEQ ID NO:2, 9, or 10. In some aspects, the gapmer comprises a sequence having 80% sequence identity to SEQ ID NO:2. In certain aspects, the gapmer comprises SEQ ID NO: 1, 2, 3, 4, 5, 6, 7, 8, 9, or 10. In certain aspects, the gapmer comprises SEQ ID NO: 2, 9, or 10. In particular aspects, the gapmer comprises SEQ ID NO:2. In specific aspects, the gapmer comprises SEQ ID NO: 14, 23, 24, 25, 41, 48, 59, or 64.

In some aspects, the gapmer comprises at least one 2′ sugar modification. In certain aspects, the gapmer comprises at least one 2′-O-methyl (2′-O-Me), 2′-O-methoxyethyl (2′-O-MOE), or 2′-fluoro (2′-F) modification.

In certain aspects, the composition is formulated as a pharmaceutical composition, such as a pharmaceutical composition formulated as liposomes or nanoparticles. In certain aspects, the nanoparticle formulation comprises brain-targeted nanoparticles, such as brain-targeted nanoparticles capable of crossing the blood brain barrier. In some aspects, the nanoparticles are dual-targeted and dual-responsive nanoparticles (DTDRN). In certain aspects, the brain-targeted nanoparticles are conjugated to scopine and/or glutathione.

Another embodiment provides the use of the composition of present embodiments and aspects thereof (e.g., a composition comprising an antisense gapmer oligonucleotide, wherein the gapmer oligonucleotide is 11-25 nucleotide units in length and able to recruit RNaseH when hybridized to Fli-1) for the treatment of an inflammatory disease. In certain aspects, the inflammatory disease is Alzheimer's disease. In some aspects, the inflammatory disease is systemic lupus erythematosus, sepsis, or multiple sclerosis.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1E: Fli-1 levels are increased and pericyte number is decreased in the hippocampus from postmortem brains of AD patients. (FIG. 1A) RNAs were isolated from 100 mg brain hippocampal or superior temporal tissue from human donors with AD (N=11) and cognitively normal controls (N=8), and Fli-1 mRNA levels were determined by RT-PCR. (FIG. 1B) Representative fluorescent images of brain hippocampus stained for Fli-1 (green) and nuclei (DAPI, blue) in human donors with AD (N=10) and cognitively normal controls (N=10). Scale bar: 60 μm. Fli-1 levels were analyzed. (FIG. 1C) Representative fluorescent images of brain hippocampus stained for Fli-1 (green), pericytes (CD13, red) and nuclei (DAPI, blue) in human donors with AD (N=10) and cognitively normal controls (N=10). Scale bar: 60 μm. Representative Fli-1⁺ pericytes were indicated by arrows and Fli-1⁺ pericyte numbers were analyzed. (FIG. 1D) Representative fluorescent images of brain hippocampus stained for pericytes (CD13, red) and nuclei (DAPI, blue) in human donors with AD (N=10) and cognitively normal controls (N=10). Scale bar: 60 μm. CD13⁺ pericyte numbers were analyzed. (FIG. 1E) Representative fluorescent images of brain hippocampus stained for active caspase-3 (green), pericytes (CD13, red) and nuclei (DAPI, blue) in human donors with AD (N=10) and cognitively normal controls (N=10). Scale bar: 60 μm. Representative active caspase-3⁺ pericytes were indicated by arrows and active caspase-3⁺ pericyte numbers were analyzed. Data are expressed as mean±standard error of the mean. *P<0.05 compared to CON group. CON: control; AD: alzheimer's disease.

FIGS. 2A-2G: Up-regulated Fli-1 levels are associated with increased pericyte loss in the hippocampus of 5×FAD mice. The hippocampus was isolated from WT and 5×FAD mice at 6.5 months of age. (FIG. 2A) Fli-1 mRNA and (FIG. 2B) Fli-1 protein levels were determined by RT-PCR and Western blot. N=5 mice/group. (FIG. 2C) Representative fluorescent images of brain hippocampus stained for Fli-1 (green) and nuclei (DAPI, blue). Scale bar: 50 μm. Quantification analysis of Fli-1 fluorescence intensity was performed. N=18 random fields from 3 mice/group. (FIG. 2D) Expression levels of TNFα, IL-6 and MMP3 were determined by RT-PCR. N=5 mice/group. (FIG. 2E) Representative fluorescent images of brain hippocampus stained for Fli-1 (green), pericytes (CD13, red) and nuclei (DAPI, blue). Scale bar: 150/50 μm. Representative Fli-1⁺ pericytes were indicated by arrows. (FIGS. 2F-2G) Fli-1⁺ pericyte numbers and CD13⁺ pericyte numbers were analyzed. N=18 random fields from 3 mice/group. Data are expressed as mean±standard error of the mean. *P<0.05 compared to WT group.

FIGS. 3A-3E: Inhibition of Fli-1 by Fli-1 Gapmer via intrahippocampal injection attenuates cognitive deficits in 5×FAD mice. Fli-1 or control Gapmers were injected into both sides of the hippocampus of 5×FAD mice at 3 and 4.5 months of age. (FIG. 3A) The expression of Fli-1 in the hippocampus was determined by RT-PCR and western blot in WT and 5×FAD mice at 6.5 months of age. N=3-5 mice/per group. Novel Object Recognition (NOR) and Morris Water Maze (MWM) tests were performed on wild type (N=8), 5×FAD mice (N=8), and 5×FAD mice injected with control (N=6) or Fli-1 Gapmers (N=7-8) at 6 months of age. (FIGS. 3B-3C) The frequency and time of recognition of novel object index (frequency to visit and time spent with novel object vs both objects) were recorded and analyzed. (FIGS. 3D-3E) The average time for mice to find the hidden platform was recorded and analyzed. Data are expressed as mean±standard error of the mean. *P<0.05 compared to WT group or 5×FAD+Con Gapmer group. ^{#}P<0.05 compared to 5×FAD+Con Gapmer group.

FIGS. 4A-4F: Inhibition of Fli-1 by Fli-1 Gapmer via intrahippocampal injection ameliorates pericyte loss and BBB dysfunction in the hippocampus of 5×FAD mice. Fli-1 or control Gapmers were injected into both sides of the hippocampus of 5×FAD mice at 3 and 4.5 months of age. The hippocampus was isolated from WT and 5×FAD mice at 6.5 months of age. (FIG. 4A) TNFα, IL-6, IL-10 and MMP3 levels were determined by RT-PCR. N=5 mice/group. FIG. 4 (FIG. 4B) Representative fluorescent images of brain hippocampus stained for Fli-1 (green), pericytes (CD13, red) and nuclei (DAPI, blue). Scale bar: 150/50 μm. Representative Fli-1⁺ pericytes were indicated by arrows. (FIGS. 4C-4D) Fli-1⁺ pericyte numbers and CD13⁺ pericyte numbers were analyzed. N=18 random fields from 3 mice/group. (FIG. 4E) Representative fluorescent images of brain hippocampus stained for IgG (red) and CD31 (green). Scale bar: 25 μm. (FIG. 4F) Quantitative analysis of IgG fluorescence intensity. N=18 random fields from 3 mice/group. Data are expressed as mean±standard error of the mean. *P<0.05 compared to 5×FAD+Con Gapmer group. ^{#}P<0.05 compared to WT group.

FIGS. 5A-5I: Inhibition of Fli-1 by Fli-1 Gapmer via intrahippocampal injection reduces Aβ accumulation in the hippocampus of 5×FAD mice. (FIG. 5A) Representative fluorescent images of brain hippocampus from WT and 5×FAD mice at 6.5 months of age stained for Aβ plaque (Thioflavin-S, green). Scale bar: 100 μm. Fli-1 or control Gapmers were injected into both sides of the hippocampus of 5×FAD mice at 3 and 4.5 months of age. The hippocampus was isolated from 5×FAD mice treated with control or Fli-1 gapmers at 6.5 months of age. (FIG. 5B) Representative fluorescent images of brain hippocampus stained for Aβ plaque (Thioflavin-S, green). Scale bar: 50 μm. (FIGS. 5C-5D) Aβ plaque number and immunoreactive area was analyzed. N=19-24 random fields from 3 mice/group. (FIG. 5E) Representative fluorescent images of brain hippocampus from WT and 5×FAD mice at 6.5 months of age stained for human Aβ (82E1, red) and nuclei (DAPI, blue). Scale bar: 50 μm. (FIG. 5F) Representative fluorescent images of brain hippocampus from 5×FAD mice treated with control or Fli-1 Gapmers stained for human Aβ (82E1, red) and nuclei (DAPI, blue). Scale bar: 50 μm. (FIG. 5G) Human Aβ immunoreactive area was analyzed. N=19 random fields from 3 mice/group. (FIG. 5H) Soluble human Aβ40, (FIG. 5I) Soluble human Aβ42 levels in the hippocampus was detected by ELISA. N=5 mice/group. Data are expressed as mean±standard error of the mean. *P<0.05 compared to 5×FAD+Con Gapmer group. ^{#}P<0.05 compared to WT group.

FIGS. 6A-6G: Inhibition of Fli-1 by Fli-1 Gapmer reduces Aβ accumulation-induced pericyte apoptosis and caspase-3 expression in human brain pericytes. Human brain pericytes were stimulated with freshly aggregated amyloid-β (1-40, 10 μM) or human TNFα (10 ng/ml) for 12 h. (FIG. 6A) Fli-1 levels were determined by RT-PCR. N=3 independent experiments. Human brain pericytes were transfected with Fli-1 or Con Gapmers for 48 h, and treated with freshly aggregated Aβ (1-40, 10 μM) for 5 or 7 consecutive days. (FIG. 6B) Fli-1 levels were determined by RT-PCR. N=3 independent experiments. (FIG. 6C) Cell viability was evaluated by PrestoBlue™ Cell Viability Reagent. Results are expressed as percentage of Con Gapmer alone group. N=12 replicates from four independent experiments. (FIG. 6D) Caspase-3 mRNA levels were measured by RT-PCR. N=3 independent experiments. (FIG. 6E) Representative fluorescent images of brain pericytes stained for TUNEL (green) or active caspase-3 (red). Scale bar: 100 μm. Quantification analysis of (FIG. 6F) TUNEL fluorescence intensity and (FIG. 6G) active caspase-3 expression were shown. N=10 random fields from three independent experiments. Data are expressed as mean±standard error of the mean. *P<0.05 compared to control or Con Gapmer group. ^{#}P<0.05 compared to Aβ40+Con Gapmer group. Con: control.

FIG. 7: Mechanism by which Fli-1 regulates pericyte loss, BBB function, and Aβ accumulation and brain hypoperfusion in AD. Aβ induces increased Fli-1 levels in pericytes, which drive caspase ⅓ expression, leading to pericyte loss and BBB dysfunction. BBB dysfunction results in inflammatory monocyte infiltration, which contribute to neuro-inflammation. Neuro-inflammation leads to Aβ accumulation, adhesion molecule expression, neutrophil arrest in capillaries and brain hypoperfusion.

FIGS. 8A-8D: Fli-1 Gapmer suppresses Fli-1 levels in both human and mouse pericytes. Human brain pericytes (FIG. 8A), human lung pericytes (FIG. 8B) and mouse brain pericytes (FIGS. 8C, 8D) were transfected with Fli-1 Gapmers or mFli-1 Gapmer for 48 hr and Fli-1 mRNA levels were determined. *p<0.05 compared to control Gapmer group.

FIGS. 9A-9D: Fli-1 Gapmer suppresses Fli-1 levels in both human and mouse pericytes. Human brain pericytes (FIGS. 9A, 9B) and mouse lung pericytes (FIGS. 9C, 9D) were transfected with Fli-1 Gapmer 2 for 24 hr and Fli-1 protein levels were determined. *p<0.05 compared to control Gapmer group. N=3.

FIG. 10: Details of human Fli-1 Gapmer Design. Human Fli-1 Gapmer 2 sequence (SEQ ID NO:2) and structure is shown.

FIGS. 11A-11C: Different Fli-1 Gapmer 2 modification exert different effectiveness in human and mouse pericytes. Human brain pericytes (FIG. 11A), human lung pericytes (FIG. 11B) and mouse brain pericytes (FIG. 11C) were transfected with Fli-1 Gapmer 2 with 2′-O-Me or different position of LNA modification for 48 hr and Fli-1 mRNA levels were determined. *p<0.05 compared to control Gapmer group.

FIGS. 12A-12F: Expression of TNFα, IL-6 and MMP3 in AD patients. RNAs were isolated from 100 mg brain hippocampal tissue or superior temporal tissue of human donors with AD (N=11) and cognitively normal controls (N=8). The expression levels of TNFα, IL-6 and MMP3 were determined by RT-PCR in the (FIGS. 12A-12C) hippocampus and (FIGS. 12D-12F) superior temporal. Data are expressed as mean±standard error of the mean. *P<0.05 compared to CON group. CON: control; AD: Alzheimer's disease.

FIGS. 13A-13D: Increased Fli-1 along with increased TNFα, IL-6 and MMP3 in the cortex of 5×FAD mice. The cortex was isolated from WT and 5×FAD mice at 6.5 months of age. (FIG. 13A) Fli-1 mRNA levels were determined by RT-PCR. N=5 mice/group. Expression levels of (FIG. 13B) TNFα, (FIG. 13C) IL-6 and (FIG. 13D) MMP3 were determined by RT-PCR. N=5 mice/group. Data are expressed as mean±standard error of the mean. *P<0.05 compared to WT group.

FIG. 14: The experimental design in vivo. The in vivo experimental design including the timeline of intrahippocampal injection of Control or Fli-1 Gapmers, behavioral test, RT-PCR, western blot, immunostaining and ELISA.

FIGS. 15A-15F: Intrahippocampal injection of Fli-1 Gapmer didn't affect expression levels of Fli-1, TNFα, IL-6 and MMP3, Aβ40, and Aβ42 levels in the cortex of 5×FAD mice. Fli-1 or control Gapmers were injected into both sides of the hippocampus of 5×FAD mice at 3 and 4.5 months of age. The cortex was isolated from WT and 5×FAD mice at 6.5 months of age. (FIG. 15A) Fli-1, (FIG. 15B) TNFα, (FIG. 15C) IL-6 and (FIG. 15D) MMP3 levels were determined by RT-PCR. (FIG. 15E) Soluble human Aβ40 levels and (FIG. 15F) Soluble human Aβ42 levels in the cortex was detected by ELISA. N=5 mice/group. Data are expressed as mean±standard error of the mean. *P<0.05 compared to WT group.

FIGS. 16A-16B: Fli-1 Gapmer treatment reduces BACE1 levels in the hippocampus of 5×FAD mice. Fli-1 or control Gapmers were injected into both sides of the hippocampus of 5×FAD mice at 3 and 4.5 months of age. BACE1 levels in the hippocampus of in WT and 5×FAD mice (FIG. 16A), and 5×FAD mice treated with control or Fli-1 Gapmers (FIG. 16B) were determined at 6.5 months of age. *p<0.05 compared to control Gapmer treated group. N=5 mice/group

FIGS. 17A-17D: ICAM1 and VCAM1 levels were higher in the hippocampus in 5×FAD mice, and they were suppressed in the Fli-1 Gapmer treatment group. The hippocampus was isolated from WT and 5×FAD mice at 6.5 months of age, and ICAM1 (FIG. 17A) and VCAM1 (FIG. 17B) mRNA levels were determined. *p<0.05 compared to WT group. N=5 mice/group. Fli-1 or control Gapmers were injected into both sides of the hippocampus of 5×FAD mice. Mice were sacrificed at 6.5 months of age and ICAM1 (FIG. 17C) and VCAM1 (FIG. 17D) levels in the hippocampus were determined. *p<0.05 compared to control Gapmer treated group. N=5 mice/group.

FIGS. 18A-18B: Intrathecal injection of Fli-1 Gapmer decrease Fli-1 levels in the brain. 5 nmol/kg of Fli-1 Gapmer-FAM were injected into the intrathecal space in WT mice. Brain sections were analyzed at 24 h after injection. The FAM-labeled Fli-1 Gapmer (green) around nuclei (DAPI, blue) were observed in both hippocampus and cortex (FIG. 18A). 5 nmol/kg of Fli-1 Gapmer were injected into the intrathecal space in WT mice. Fli-1 levels in the hippocampus and cortex were determined at 7 days after injection (FIG. 18B). p<0.05, N=4-6 mice/group.

FIGS. 19A-19C: Intrathecal Fli-1 Gapmer treatment ameliorated spatial learning and memory impairment in 5×FAD mice. Fli-1 or control Gapmers were injected into intrathecal space of 5×FAD mice at 3, 4, and 5 months of age. Novel Object Recognition (NOR) test was performed on 5×FAD mice injected with control (N=9) or Fli-1 (N=9) Gapmers at 6 months of age. The frequency (FIG. 19A) and time (FIG. 19B) of recognition of novel object index were recorded and analyzed. Morris Water Maze (MWM) tests were performed on 5×FAD mice injected with control (N=10) or Fli-1 (N=11) Gapmers at 6 months of age and the average time for mice to find the hidden platform (FIG. 19C) was recorded and analyzed. *p<0.05 compared control Gapmer treated group.

### DESCRIPTION OF ILLUSTRATIVE EMBODIMENTS

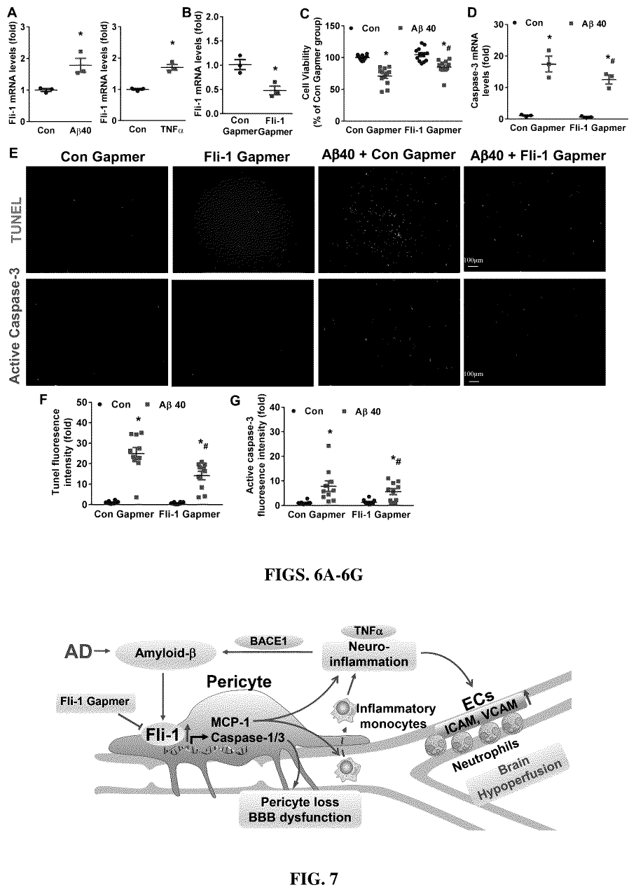

Alzheimer's disease (AD) pathology is characterized by cerebral plaques containing aggregates of various amyloid-β (Aβ) peptides derived from amyloid precursor protein (APP), as well as by neurofibrillary tangles (NFTs) containing hyperphosphorylated and aggregated tau protein. However, it is now recognized that vascular dysfunction, such as decreases in cerebral blood flow (CBF) and disruption of the blood brain barrier (BBB), could contribute to and precede AD onset and progression (Kisler et al., 2017) (Iadecola et al., 2019). In the brain, specialized cells called pericytes are integral to proper vascular function, as they play a major role in regulating CBF and maintaining BBB integrity (Brown et al., 2019). Most importantly, pericytes also play a critical role in the removal of Aβ (Ma et al., 2018). Thus, pericyte functions are increasingly recognized as paramount to healthy brain activity, and dysfunction may underlie or exacerbate neurodegenerative diseases such as AD (Brown et al., 2019). Elucidating the mechanisms by which pericyte dysfunction leads to cognitive dysfunction in AD will result in a better understanding of the pathogenesis of AD and the development of a novel treatment strategy for AD (FIG. 7).

Brain pericytes regulate cerebral blood flow, maintain the integrity of the blood-brain barrier (BBB) and facilitate the removal of amyloid β (Aβ) which is critical to healthy brain activity. Pericyte loss has been observed in brains from patients with Alzheimer's disease (AD) and animal models. It has been demonstrated that friend leukemia virus integration 1 (Fli-1), an ETS transcription factor, governs pericyte viability in murine sepsis; however, the role of Fli-1 and its impact on pericyte loss in AD remains unknown.

Fli-1 is a member of the ETS transcription factor family that was first identified in erythroleukemias induced by Friend Murine Leukemia Virus (F-MuLV). Fli-1 is activated through retroviral insertional mutagenesis in 90% of F-MuLV-induced erythroleukemias. The constitutive activation of Fli-1 in erythroblasts leads to a dramatic shift in the Epo/Epo-R signal transduction pathway, blocking erythroid differentiation, activating the Ras pathway, and resulting in massive Epo-independent proliferation of erythroblasts. These results suggest that Fli-1 overexpression in erythroblasts alters their responsiveness to Epo and triggers abnormal proliferation by switching the signaling event(s) associated with terminal differentiation to proliferation.

In the present studies, the potential mechanisms by which Fli-1 may modulate pericyte stability within the BBB and contribute to AD pathogenesis were determined. It was found that Fli-1 expression is increased in the brain, including within pericytes from patients with AD and 5×FAD mice. Specifically, it was demonstrated that Fli-1 expression was up-regulated in postmortem brains from a cohort of human AD donors and in 5×FAD mice, which corresponded with a decreased pericyte number, elevated inflammatory mediators TNFα and IL-6, and increased Aβ accumulation as compared to cognitively normal individuals and WT mice. It was further shown that inhibition of Fli-1 by an antisense oligonucleotide Gapmer administrated via intrahippocampal injection retards pericyte loss, ameliorates cognitive deficits, reduces Aβ deposition, and decelerates BBB breakdown in 5×FAD mice. Knockdown of Fli-1 prevented Aβ accumulation-induced human brain pericyte apoptosis in vitro. Suppression of Fli-1 with gapmers ameliorated Aβ deposition, inflammation, BBB breakdown, and spatial learning and memory impairment in AD. Thus, Fli-1 contributes to pericyte degeneration and cognitive impairment in AD and provides a novel therapeutic target to modify AD progression.

Accordingly, in certain embodiments, the present disclosure provides methods of treating inflammatory diseases, such as AD, by inhibiting Fli-1 in a subject with AD. Fli-1 may be inhibited by antisense oligonucleotides, such as locked nucleic acid (LNA) gapmers, or with Fli-1 antibodies. In particular aspects, Fli-1 is inhibited by a gapmer having a sequence of SEQ ID NO: 2. In some aspects, the Fli-1 antisense oligonucleotide may be administered to the brain by liposomes, such as by intrathecal delivery, or nanoparticles (e.g., brain-targeting nanoparticles), such as by intravenous administration. The nanoparticles may be dual-targeted and dual-responsive nanoparticles (DTDRN) as described in U.S. Patent Application No. US20160346208A1; incorporated herein by reference in its entirety. In other aspects, compounds that inhibit Fli-1 transcriptional activity are used to prevent and/or treat AD development, such as diterpenoid-like compound described in Liu et al., 2019; incorporated herein by reference in its entirety. Further, Fli-1 inhibition as described herein may be used to treat diseases, such as cancer or systemic lupus erythematosus.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “about” means, in general, within a standard deviation of the stated value as determined using a standard analytical technique for measuring the stated value. The terms can also be used by referring to plus or minus 5% of the stated value.

The phrase “effective amount” or “therapeutically effective” means a dosage of a drug or agent sufficient to produce a desired result. The desired result can be subjective or objective improvement in the recipient of the dosage, increased lung growth, increased lung repair, reduced tissue edema, increased DNA repair, decreased apoptosis, a decrease in tumor size, a decrease in the rate of growth of cancer cells, a decrease in metastasis, or any combination of the above.

“Subject” and “patient” refer to either a human or non-human, such as primates, mammals, and vertebrates. In particular embodiments, the subject is a human.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a tumor or malignancy, delay or slowing of tumor growth and/or metastasis, and an increased lifespan as compared to that expected in the absence of treatment.

As used herein, “nucleoside” means a compound comprising a nucleobase moiety and a sugar moiety. Nucleosides include, but are not limited to, naturally occurring nucleosides (as found in DNA and RNA) and modified nucleosides. Nucleosides may be linked to a phosphate moiety.

As used herein, “chemical modification” means a chemical difference in a compound when compared to a naturally occurring counterpart. Chemical modifications of oligonucleotides include nucleoside modifications (including sugar moiety modifications and nucleobase modifications) and internucleoside linkage modifications. In reference to an oligonucleotide, chemical modification does not include differences only in nucleobase sequence.

As used herein, “nucleotide” means a nucleoside further comprising a phosphate linking group. As used herein, “linked nucleosides” may or may not be linked by phosphate linkages and thus includes but is not limited to “linked nucleotides.” As used herein, “linked nucleosides” are nucleosides that are connected in a continuous sequence (i.e., no additional nucleosides are present between those that are linked).

As used herein, “nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Nucleobases may be naturally occurring or may be modified.

As used herein the terms, “unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methyl C), and uracil (U).

As used herein, “modified nucleobase” means any nucleobase that is not a naturally occurring nucleobase.

As used herein, “modified nucleoside” means a nucleoside comprising at least one chemical modification compared to naturally occurring RNA or DNA nucleosides. Modified nucleosides comprise a modified sugar moiety and/or a modified nucleobase.

As used herein, “locked nucleic acid nucleoside” or “LNA” means a nucleoside comprising a bicyclic sugar moiety comprising a 4′-CH₂—O-2′ bridge.

As used herein, “2′-substituted nucleoside” means a nucleoside comprising a substituent at the 2′-position other than H or OH. Unless otherwise indicated, a 2′-substituted nucleoside is not a bicyclic nucleoside.

As used herein, “2′-deoxynucleoside” means a nucleoside comprising 2′-H furanosyl sugar moiety, as found in naturally occurring deoxyribonucleosides (DNA). In certain embodiments, a 2′-deoxynucleoside may comprise a modified nucleobase or may comprise an RNA nucleobase (e.g., uracil).

As used herein, “phosphorous moiety” refers to a to monovalent P(V) phosphorus radical group. In certain embodiments, a phosphorus moiety is selected from: a phosphate, phosphonate, alkylphosphonate, vinylphosphonate, aminoalkyl phosphonate, phosphorothioate, phosphoramidite, alkylphosphonothioate, phosphorodithioate, thiophosphoramidate, phosphotriester and the like.

The term “phosphate moiety” as used herein, refers to a terminal phosphate group that includes unmodified phosphates (—O—P(═O)(OH)OH) as well as modified phosphates. Modified phosphates include but are not limited to phosphates in which one or more of the O and OH groups is replaced with H, O, S, N(R), alkyl, alkenyl, or alkynl where R is H, an amino protecting group or unsubstituted or substituted alkyl.

As used herein, “phosphate stabilizing modification” refers to a modification that results in stabilization of a 5′-phosphate moiety of the 5′-terminal nucleoside of an oligonucleotide, relative to the stability of an unmodified 5′-phosphate of an unmodified nucleoside under biologic conditions. Such stabilization of a 5′-phophate group includes but is not limited to resistance to removal by phosphatases. Phosphate stabilizing modifications include, but are not limited to, modification of one or more of the atoms that binds directly to the phosphorus atom, modification of one or more atoms that link the phosphorus to the 5′-carbon of the nucleoside, and modifications at one or more other positions of the nucleoside that result in stabilization of the phosphate. In certain embodiments, a phosphate stabilizing modification comprises a carbon linking the phosphorous atom to the 5′-carbon of the sugar. Phosphate moieties that are stabilized by one or more phosphate stabilizing modification are referred to herein as “stabilized phosphate moieties.”

As used herein, “oligonucleotide” means a compound comprising a plurality of linked nucleosides. In certain embodiments, an oligonucleotide comprises one or more unmodified ribonucleosides (RNA) and/or unmodified deoxyribonucleosides (DNA) and/or one or more modified nucleosides.

As used herein “oligonucleoside” means an oligonucleotide in which none of the internucleoside linkages contains a phosphorus atom. As used herein, oligonucleotides include oligonucleosides.

As used herein, “modified oligonucleotide” means an oligonucleotide comprising at least one modified nucleoside and/or at least one modified internucleoside linkage.

As used herein “internucleoside linkage” means a covalent linkage between adjacent nucleosides in an oligonucleotide.

As used herein “naturally occurring internucleoside linkage” means a 3′ to 5′ phosphodiester linkage.

As used herein, “modified internucleoside linkage” means any internucleoside linkage other than a naturally occurring internucleoside linkage.

As used herein, “oligomeric compound” means a polymeric structure comprising two or more sub-structures. In certain embodiments, an oligomeric compound comprises an oligonucleotide. In certain embodiments, an oligomeric compound comprises one or more conjugate groups and/or terminal groups. In certain embodiments, an oligomeric compound consists of an oligonucleotide. Oligomeric compounds also include naturally occurring nucleic acids.

As used herein, “terminal group” means one or more atom(s) attached to either, or both, the 3′ end or the 5′ end of an oligonucleotide. In certain embodiments a terminal group is a conjugate group. In certain embodiments, a terminal group comprises one or more terminal nucleosides.

As used herein, “conjugate” means an atom or group of atoms bound to an oligonucleotide or oligomeric compound. In general, conjugate groups modify one or more properties of the compound to which they are attached, including, but not limited to pharmacodynamic, pharmacokinetic, binding, absorption, cellular distribution, cellular uptake, charge and/or clearance properties.

As used herein, “conjugate linking group” means any atom or group of atoms used to attach a conjugate to an oligonucleotide or oligomeric compound.

As used herein, “single-stranded” means an oligomeric compound that is not hybridized to its complement and which lacks sufficient self-complementarity to form a stable self-duplex.

As used herein, “antisense compound” means a compound comprising or consisting of an oligonucleotide at least a portion of which is complementary to a target nucleic acid to which it is capable of hybridizing, resulting in at least one antisense activity.

As used herein, “antisense activity” means any detectable and/or measurable change attributable to the hybridization of an antisense compound to its target nucleic acid. In certain embodiments, antisense activity is a reduction of a gain-of-function of an expanded repeat-containing nucleic acid. In certain embodiments, antisense activity is a correction of a loss-of-function of an expanded repeat-containing nucleic acid.

As used herein, “detecting” or “measuring” means that a test or assay for detecting or measuring is performed. Such detection and/or measuring may result in a value of zero. Thus, if a test for detection or measuring results in a finding of no activity (activity of zero), the step of detecting or measuring the activity has nevertheless been performed.

As used herein, “detectable and/or measureable activity” means a statistically significant activity that is not zero.

As used herein, “essentially unchanged” means little or no change in a particular parameter, particularly relative to another parameter which changes much more. In certain embodiments, a parameter is essentially unchanged when it changes less than 5%. In certain embodiments, a parameter is essentially unchanged if it changes less than two-fold while another parameter changes at least ten-fold. For example, in certain embodiments, an antisense activity is a change in the amount of a target nucleic acid. In certain such embodiments, the amount of a non-target nucleic acid is essentially unchanged if it changes much less than the target nucleic acid does, but the change need not be zero.

As used herein, “expression” means the process by which a gene ultimately results in a protein. Expression includes, but is not limited to, transcription, post-transcriptional modification (e.g., splicing, polyadenylation, addition of 5′-cap), and translation.

As used herein, “target nucleic acid” means a nucleic acid molecule to which an antisense compound hybridizes.

As used herein, “targeting” or “targeted to” means the association of an antisense compound to a particular target nucleic acid molecule or a particular region of a target nucleic acid molecule. An antisense compound targets a target nucleic acid if it is sufficiently complementary to the target nucleic acid to allow hybridization under physiological conditions.

As used herein, the term “expanded repeat-containing RNA” means a mutant RNA molecule having a nucleobase sequence that includes a repeat region having a predetermined number of nucleobases repeats, wherein the presence or length of the repeat region affects the normal processing, function, or activity of the RNA or corresponding protein.

As used herein, the term “corresponding wild-type RNA” means the non-mutant version of the expanded repeat-containing RNA having normal function and activity. Typically, corresponding wild-type RNA molecules comprise a repeat region which is shorter than that of an expanded repeat-containing RNA.

As used herein, “selectivity” refers to the ability of an antisense compound to exert an antisense activity on a target nucleic acid to a greater extent than on a non-target nucleic acid.

As used herein, “nucleobase complementarity” or “complementarity” when in reference to nucleobases means a nucleobase that is capable of standard Watson-Crick base pairing with another nucleobase. For example, adenine (A) is complementary to thymine (T) or uracil (U), and cytosine is complementary to guanine. In certain embodiments, complementary nucleobase means a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and thus, are still capable of nucleobase complementarity.

As used herein, “non-complementary” in reference to nucleobases means a pair of nucleobases that do not form hydrogen bonds with one another.

As used herein, “complementary” in reference to oligomeric compounds (e.g., linked nucleosides, oligonucleotides, or nucleic acids) means the capacity of such oligomeric compounds or regions thereof to hybridize to another oligomeric compound or region thereof through nucleobase complementarity. Complementary oligomeric compounds need not have nucleobase complementarity at each nucleoside. Rather, some mismatches are tolerated. In certain embodiments, complementary oligomeric compounds or regions are complementary at 70% of the nucleobases (70% complementary). In certain embodiments, complementary oligomeric compounds or regions are 80% complementary. In certain embodiments, complementary oligomeric compounds or regions are 90% complementary. In certain embodiments, complementary oligomeric compounds or regions are 95% complementary. In certain embodiments, complementary oligomeric compounds or regions are 100% complementary.

As used herein, “mismatch” means a nucleobase of a first oligomeric compound that is not capable of pairing with a nucleobase at a corresponding position of a second oligomeric compound, when the first and second oligomeric compound are aligned. Either or both of the first and second oligomeric compounds may be oligonucleotides.

As used herein, “hybridization” means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleobases.

As used herein, “specifically hybridizes” means the ability of an oligomeric compound to hybridize to one nucleic acid site with greater affinity than it hybridizes to another nucleic acid site.

As used herein, “fully complementary” in reference to an oligonucleotide or portion thereof means that each nucleobase of the oligonucleotide or portion thereof is capable of pairing with a nucleobase of a complementary nucleic acid or contiguous portion thereof. Thus, a fully complementary region comprises no mismatches or unhybridized nucleobases in either strand.

As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

As used herein, “percent identity” means the number of nucleobases in a first nucleic acid that are the same type (independent of chemical modification) as nucleobases at corresponding positions in a second nucleic acid, divided by the total number of nucleobases in the first nucleic acid.

As used herein, “modulation” means a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression. As a further example, modulation of expression can include a change in splice site selection of pre-mRNA processing, resulting in a change in the absolute or relative amount of a particular splice-variant compared to the amount in the absence of modulation.

As used herein, “motif” means a pattern of chemical modifications in an oligonucleotide or a region thereof. Motifs may be defined by modifications at certain nucleosides and/or at certain linking groups of an oligonucleotide.

As used herein, “nucleoside motif” means a pattern of nucleoside modifications in an oligonucleotide or a region thereof. The linkages of such an oligonucleotide may be modified or unmodified. Unless otherwise indicated, motifs herein describing only nucleosides are intended to be nucleoside motifs. Thus, in such instances, the linkages are not limited.

As used herein, “sugar motif” means a pattern of sugar modifications in an oligonucleotide or a region thereof.

As used herein, “linkage motif” means a pattern of linkage modifications in an oligonucleotide or region thereof. The nucleosides of such an oligonucleotide may be modified or unmodified. Unless otherwise indicated, motifs herein describing only linkages are intended to be linkage motifs. Thus, in such instances, the nucleosides are not limited.

As used herein, “nucleobase modification motif” means a pattern of modifications to nucleobases along an oligonucleotide. Unless otherwise indicated, a nucleobase modification motif is independent of the nucleobase sequence.

As used herein, “sequence motif” means a pattern of nucleobases arranged along an oligonucleotide or portion thereof. Unless otherwise indicated, a sequence motif is independent of chemical modifications and thus may have any combination of chemical modifications, including no chemical modifications.

As used herein, “type of modification” in reference to a nucleoside or a nucleoside of a “type” means the chemical modification of a nucleoside and includes modified and unmodified nucleosides. Accordingly, unless otherwise indicated, a “nucleoside having a modification of a first type” may be an unmodified nucleoside.

By the term “derivatives thereof” in connection with nucleotides (e.g., LNA and derivatives thereof) is understood that the nucleotide, in addition to the bridging of the furan ring, can be further derivatized. For example, the base of the nucleotide, in addition to adenine, guanine, cytosine, uracil and thymine, can be a derivative thereof, or the base can be substituted with other bases. Such bases includes heterocyclic analogues and tautomers thereof. Illustrative examples of nucleobases are xanthine, diaminopurine, 8-oxo-N6-methyladenine, 7-deazaxanthine, 7-deazaguanine, N41N4-ethanocytosin, N61N6-ethano-2,6-diaminopurine, 5-methylcytosine, 5-(C3-C6)-alkynylcytosine, 5-fluorouracil, 5-bromouracil, pseudoisocytosine, 2-hydroxy-5-methyl-4-triazolopyridin, isocytosine, isoguanin, inosine, N6-alylpurines, N6-acylpurines, N6-benzylpurine, N6-halopurine, N6-vinylpurine, N6-acetylenic purine, N6-acyl purine, 1V6-hydroxyalkyl purine, 1V6-thioalkyl purine, N2-alkylpurines, N4-alkylpyrimidines, acylpyrimidines, N4-benzylpurine, /V4-halopyrimidines, N4-vinylpyrimidines, /1/4-acetylenic pyrimidines, N4-acyl pyrimidines, N4-hydroxyalkyl pyrimidines, N6-thioalkyl pyrimidines, thymine, cytosine, 6-azapyrimidine, including 6-azacytosine, 2- and/or 4-mercaptopyrimidine, uracil, C5-alkylpyrimidines, C5-benzylpyrimidines, C5-halopyrimidines, C5-vinylpyrimidine, C5-acetylenic pyrimidine, C6-acyl pyrimidine, C5-hydroxyalkyl purine, C5-amidopyrimidine, C5-cyanopyrimidine, C5-nitropyrimidine, C5-aminopyrimdine, N2-alkylpurines, N2-alkyl-6-thiopurines, azacytidinyl, trazolopyridinyl, 5-azauracilyl, imidazolopyridinyl, pyrrolopyrimidinyl, and pyrazolopyrimidinyl. Functional oxygen and nitrogen groups on the base can be protected as necessary or desired. Suitable protecting groups are well known to those skilled in the art, and included trinnethylsilyl, dimethylhexylsilyl, t-butyldimenthylsilyl, and t-butyldiphenylsilyl, trityl, alkyl groups, acyl groups such as acetyl and propionyl, methanesulfonyl, and p-toluenesulfonyl. Preferred bases include cytosine, methyl cytosine, uracil, thymine, adenine and guanine. In addition to the derivatisation of the base, both locked and non-locked nucleotides can be derivatised on the ribose moiety. For example, a 2′ substituent can be introduced, such as a substituent selected from the group consisting of halogen (such as fluor), C1-C9 alkoxy (such as methoxy, ethoxy, n-propoxy or i-propoxy), C1-C9 aminoalkoxy (such as aminomethoxy and aminoethoxy), allyloxy, imidazolealkoxy, and polyethyleneglycol, or a 5′ substituent (such as a substituent as defined above for the 2′ position) can be introduced.

By the terms “internucleoside linkage” and “linkage between the nucleotide units” (which is used interchangeably) are to be understood the divalent linker group that forms the covalent linking of two adjacent nucleosides, between the 3′ carbon atom on the first nucleoside and the 5′ carbon atom on the second nucleoside (said nucleosides being 3,5′ dideoxy). The oligonucleotides of the present disclosure comprises sequences in which both locked and non-locked nucleotides independently can be derivatized on the internucleoside linkage which is a linkage consisting of preferably 2 to 4 groups/atoms selected from —CH₂—, —O—, —S—, —NRH—, >C═O, >C═NRH, >C═S, -51(R″)2-, —SO—, —S(O)2-, —P(O)2-, —PO(Bl3)-, —P(O,S)—, —P(S)2-, —PO(R″)—, —PO(OCH3)-, and —PO(NHRH)—, where RH is selected form hydrogen and C1.6-alkyl, and R″ is selected from C1_6-alkyl and phenyl. Illustrative examples of such internucleoside linkages are —CH₂—CH₂—CH₂—, —CH₂—CO—CH₂—, —CH₂—, —O—CH₂—O—, —O—CH₂—CH₂—, —O—CH₂—CH(R5)-, —CH₂—CH₂—O—, —NRH—CH₂—CH₂—, —CH₂—NRH—, —CH₂—NRH—CH₂—, —O—CH₂—CH₂—NRH—, —NR′—CO—O—, —NRH—CO—NRH—, —NRH—CS—NRH—, —NRH—C(═NRH)—NRH—, —NRH—CO—CH2-NRH—, —O—OO—O—, —O—CO—CH2-O—, —O—CH₂—OO—O—, —CO—NRH—, —O—CO—NRH—, —NRH—CO—CH₂—, —O—CH₂—CO—NRH—, —O—CH₂—CH₂—NRH—, —CH═N—O—, —CH₂—NRH—O—, —CH₂—O—N(R5)-, —CH₂—O—NRH—, —CO—NRH—CH₂—, —CH₂—NRH—O—, —CH2-NRH—OO—, —O—NRH—CH2-, —O—NRH—, —O—CH₂—S—, —S—CH₂—O—, —CH₂—CH2-S—, —O—CH₂—CH₂—S—, —S—CH₂—CH(R5)-, —S—CH₂—CH₂—, —S—CH₂—CH₂—O—, —S—CH₂—CH₂—S—, —CF12-S—CF12-, —CH₂—SO—CH₂—, —CH₂—SO₂—CH₂—, —O—SO—O—, —O—S(O)2-O—, —O-5(O)2-CH₂—, —O—S(O)2-NRH—, —NRH—S(O)2-CH₂—, —O—S(O)2-CH₂—, —O—P(O)2-O—, —O—P(O,S)—O—, —O—P(S)2-O—, —S—P(O)2-O—, —S—P(O,S)—O—, —S—P(S)2-O—, —O—P(O)2-S—, —O—P(O,S)—S—, —O—P(S)2-S—, —S—P(O)2-S—, —S—P(O,S)—S—, —S—P(S)2-S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR″)—O—, —O—P(O)2-NRI1-, —NR′—P(O)2-O—, —O—P(O,NRH)—O—, —P(O)2-O—, —O—P(O)2-CH₂—, and —O—Si(R″)2-O—; where R5 is selected from hydrogen and C1-6-alkyl, RH is selected form hydrogen and C1-6-alkyl, and R″ is selected from C1-6-alkyl and phenyl. —CH₂—CO—NRH—, —CH₂—NRH—O—, —S—CH₂—O—, —O—P(O,S)—O—, —NRH—P(O)2-O—, —O—P(O,NRH)—O—, —O—PO(R″)—O—, —O—PO(CH₃)—O—, and —O—PO(NHRN)—O—, where RH is selected from hydrogen and C1-6-alkyl, and R″ is selected from C1-5-alkyl and phenyl, are especially preferred. The nucleotides units may also contain a 3′-Terminal group or a 5′-terminal group, preferably —OH.

As used herein, “differently modified” mean chemical modifications or chemical substituents that are different from one another, including absence of modifications. Thus, for example, a MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.

As used herein, “the same type of modifications” refers to modifications that are the same as one another, including absence of modifications. Thus, for example, two unmodified DNA nucleosides have “the same type of modification,” even though the DNA nucleoside is unmodified. Such nucleosides having the same type of modification may comprise different nucleobases.

As used herein, “separate regions” means portions of an oligonucleotide wherein the chemical modifications or the motif of chemical modifications of any neighboring portions include at least one difference to allow the separate regions to be distinguished from one another.

As used herein, “parenteral administration,” means administration through injection or infusion. Parenteral administration includes, but is not limited to, subcutaneous administration, intravenous administration, or intramuscular administration.

As used herein, “systemic administration” means administration to an area other than the intended locus of activity. Examples or systemic administration are subcutaneous administration and intravenous administration, and intraperitoneal administration.

As used herein, “intravenous administration” means administration into a vein.

As used herein, “intrathecal” or “IT” means administration into the CSF under the arachnoid membrane which covers the brain and spinal cord. IT injection is performed through the theca of the spinal cord into the subarachnoid space, where a pharmaceutical agent is injected into the sheath surrounding the spinal cord.

### II. FLI-1 INHIBITION

In certain aspects, the present disclosure concerns methods and compositions for the inhibition of Fli-1. The inhibition may be by oligonucleotides, such as antisense oligonucleotides, particularly gapmers. The oligonucleotides bind to target sequences, such as Fli-1.

The length of the oligonucleotide may be 10-50 bases, and more specifically 13-30 bases, such as 13, 14, 15, 16, 17, 18, 19, 20, 21, 22, 23, 24, 25, 26, 27, 28, 29, or 30 nucleobases. The oligonucleotide or oligonucleotide analog may, in particular, be RNA and include one or more modified and/or non-natural nucleobases. The oligonucleotide or oligonucleotide analog may contain DNA as well as RNA nucleobases, such as terminal thymidine residues.

The present antisense oligonucleotides may be single stranded antisense oligonucleotides, which comprise one or more 2′sugar modified nucleosides, such as one or more LNA nucleosides or one or more 2′ MOE nucleosides. The antisense oligonucleotide may be capable of modulating the expression of a target nucleic acid, such as a target pre-mRNA, mRNA, microRNA, long non-coding RNA or viral RNA, in a cell which is expressing the target RNA—in vivo or in vitro. In some embodiments, the single stranded antisense oligonucleotide further comprises phosphorothioate internucleoside linkages. The single stranded antisense oligonucleotide may, for example may be in the form of a gapmer oligonucleotide, a mixmer oligonucleotide or a totalmer oligonucleotide. The single stranded antisense oligonucleotide mixmer may be for use in modulating a splicing event in a target pre-mRNA. The single stranded antisense oligonucleotide mixmer may be for use in inhibiting the expression of a target microRNA. The single stranded antisense oligonucleotide mixmer may be for use in inhibiting the interaction between a long non-coding RNA and chromatin, thereby alleviating chromatin (such as PRC2) mediated repression of one or more mRNAs. The single stranded antisense oligonucleotide gapmer may be for inhibition of a target pre-mRNA, a target mRNA, a target viral RNA, or a target long non-coding RNA.

The oligonucleotide or oligonucleotide analog may be double-stranded or single-stranded, including ds-RNAs and ss-siRNAs. Single-stranded antisense oligonucleotides do not require the RNAi machinery and are a different strategy for silencing gene expression. Single-stranded antisense oligonucleotides may comprise locked nucleic acids (LNAs). LNA nucleotides are constrained by a bond between the 2′ and 4′ positions of the ribose ring. This constraint “locks” the nucleotide into a position that is ideal for base-pairing and the introduction of a handful of LNA nucleotides into single-stranded antisense oligonucleotides can tailor the affinity. Other modifications that may be used include but are not limited to 2′-O-methoxyethyl RNA and tricycloDNA. A person of skill in the art will understand additional modifications that can be used to tailor single-stranded antisense oligonucleotide binding affinity and nucleobase stability.

In particular, the single-stranded antisense oligonucleotides used herein may be gapmers, such as with LNA modifications at 3′ and 5′ end, clustered around a DNA gap (e.g., 8, 9, 10, 11, or 12 DNA nucleobases), which are capable of recruiting RNase H and cleavage of the RNA target. Particularly contemplated sequences include, but are not limited to, human Fli-1 Gapmer 2 sequence (SEQ ID NO:2) as shown in FIG. 11, a sequence comprising 80% sequence identity to SEQ ID NO:2, or SEQ ID NO:2 with one, two, three, four or more mutations.

Commercially available equipment routinely used for the support-media-based synthesis of oligomeric compounds and related compounds is sold by several vendors including, for example, Biolytic (Fremont, CA), Bioautomation (Irving, TX), General Electric, as well as others. Suitable solid phase techniques, including automated synthesis techniques, are described in Scozzari and Capaldi, “Oligonucleotide Manufacturing and Analytic Processes for 2′-O-(2-methoxyethyl-Modified Oligonucleotides” in Crooke, S. T. (ed.) Antisense Drug Technology: Principles, Strategies, and Applications, Second Edition (2007).

In other aspects, the inhibition of Fli-1 may comprise a small molecule compound. In some aspects, the small molecule inhibitor of Fli-1 is a diterpenoid-like compound that inhibits Fli-1 transcriptional activity, such as A661 or A665 as described in Liu et al., 2019; incorporated by reference herein in its entirety. In certain aspects, the small molecule is camptothecin (CPT) or topotecan (TPT) (Wang et al., 2021).

### A. RNA Interference

In some aspects, Fli-1 inhibition comprises RNA interference. RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity. Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene. It is generally accepted that RNAi acts post-transcriptionally, targeting RNA transcripts for degradation. It appears that both nuclear and cytoplasmic RNA can be targeted.

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 20 to 23 nucleotides in length are most effective. This length reflects the lengths of digestion products resulting from the processing of much longer RNAs as described above. siRNA and double-stranded RNA have been described in U.S. Pat. Nos. 6,506,559 and 6,573,099, as well as in U.S. Patent Applications US2003/0051263, US2003/0055020, US2004/0265839, US2002/0168707, US2003/0159161, and US2004/0064842, all of which are herein incorporated by reference in their entirety.

Several further modifications to siRNA sequences have been suggested in order to alter their stability or improve their effectiveness. It has been suggested that synthetic complementary 21-mer RNAs having di-nucleotide overhangs (i.e., 19 complementary nucleotides+3′ non-complementary dimers) may provide the greatest level of suppression. These protocols may use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang. More recently, it has been suggested that it may be preferable for the overhangs to use 2′-OMe-uridine (2′-OMe-U). It may also be preferable to use a single 3′ overhang in the antisense strand and have no overhang in the sense strand.

Short hairpin RNA (shRNA) is a sequence of RNA that makes a tight hairpin turn that can be used to silence gene expression. shRNA is transcribed by RNA polymerase III. shRNA production in a mammalian cell can sometimes cause the cell to mount an interferon response as the cell seeks to defend itself from what it perceives as viral attack. Paddison et al. (2002) examined the importance of stem and loop length, sequence specificity, and presence of overhangs in determining shRNA activity. The authors found some interesting results. For example, they showed that the length of the stem and loop of functional shRNAs could vary. Stem lengths could range anywhere from 25 to 29 nt and loop size could range between 4 to 23 nt without affecting silencing activity. Presence of G-U mismatches between the 2 strands of the shRNA stem did not lead to a decrease in potency. Complementarity between the portion of the stem that binds to the target mRNA (antisense strand) and the mRNA, on the other hand, was shown to be critical. Single base mismatches between the antisense strand of the stem and the mRNA abolished silencing. It has been reported that presence of 2 nt 3′-overhangs is critical for siRNA activity. Presence of overhangs on shRNAs, however, did not seem to be important. Some of the functional shRNAs that were either chemically synthesized or in vitro transcribed, for example, did not have predicted 3′ overhangs.

dsRNA can be synthesized using well-described methods. Briefly, sense and antisense RNA are synthesized from DNA templates using T7 polymerase (MEGAscript, Ambion). After the synthesis is complete, the DNA template is digested with DNaseI and RNA purified by phenol/chloroform extraction and isopropanol precipitation. RNA size, purity and integrity are assayed on denaturing agarose gels. Sense and antisense RNA are diluted in potassium citrate buffer and annealed at 80° C. for 3 min to form dsRNA. The sum of the individual dsRNA species is designated as a “dsRNA library.”

The making of siRNAs has been mainly through direct chemical synthesis; through processing of longer, double-stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single-stranded RNA-oligomers followed by the annealing of the two single-stranded oligomers into a double-stranded RNA.

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single-stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

Several groups have developed expression vectors that continually express siRNAs in stably transfected mammalian cells. Some of these plasmids are engineered to express shRNAs lacking poly(A) tails. Transcription of shRNAs is initiated at a polymerase III (pol III) promoter and is believed to be terminated at position 2 of a 4-5-thymine transcription termination site. shRNAs are thought to fold into a stem-loop structure with 3′ UU-overhangs. Subsequently, the ends of these shRNAs are processed, converting the shRNAs into ˜21 nt siRNA-like molecules. The siRNA-like molecules can, in turn, bring about gene-specific silencing in the transfected mammalian cells.

More generally, most any oligo- or polynucleotide may be made by any technique known to one of ordinary skill in the art, such as chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described in U.S. Pat. No. 5,705,629, each incorporated herein by reference. In the methods of the present disclosure, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

### B. Antisense Oligonucleotide

In some embodiments, Fli-1 antisense oligonucleotides are provided herein. Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G: C) and adenine paired with either thymine (A: T) or uracil (A: U). Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs may include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

### C. Gapmers

The present antisense oligonucleotide, or contiguous nucleotide sequence thereof, may be a gapmer. The antisense gapmers are commonly used to inhibit a target nucleic acid via RNase H mediated degradation. A gapmer oligonucleotide comprises at least three distinct structural regions a 5′-flank, a gap and a 3′-flank, F-G-F′ in the ‘5->3’ orientation. The “gap” region (G) comprises a stretch of contiguous DNA nucleotides which enable the oligonucleotide to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e., are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

Exemplary FLI-1 gapmer sequences are provided below.

Human Gapmer-1
(SEQ ID NO: 1)
5′-T*C*G*T*G*A*G*G*A*T*T*G*G*T*C*G-3′
Human Gapmer-2: Human gapmer targeting both
human and mouse Fli-1
(SEQ ID NO: 2)
5′-C*C*G*T*G*C*A*C*T*T*T*G*G*T*C*A-3′
Human Gapmer-3
(SEQ ID NO: 3)
5′-C*A*A*T*G*C*C*G*T*G*G*A*A*G*T*C-3′
Human Gapmer-4
(SEQ ID NO: 4)
5′-T*G*A*G*G*A*T*T*G*G*T*C*G*G*T*G-3′
Human Gapmer-5
(SEQ ID NO: 5)
5′-C*G*T*G*G*A*A*G*T*C*A*A*A*T*T*T-3′
Human Gapmer-6
(SEQ ID NO: 6)
5′-G*A*T*A*G*T*G*C*A*A*G*A*A*G*T*A-3′
Human Gapmer-7
(SEQ ID NO: 7)
5′-G*G*G*C*G*G*T*A*A*A*T*A*A*G*A*A-3′
Human Gapmer-8
(SEQ ID NO: 8)
5′-C*G*T*G*C*A*C*T*T*T*G*G*T*C*A*T-3′
Human Gapmer-9 Human gapmer targeting both
human and mouse Fli-1
(SEQ ID NO: 9)
5′-G*C*G*A*T*T*G*A*T*T*C*G*A*T*A*G-3′
Human Gapmer-10 Human gapmer targeting both
human and mouse Fli-1
(SEQ ID NO: 10)
5′-A*A*C*A*G*C*G*A*T*T*G*A*T*T*C*G-3′
Mouse Gapmer-1
(SEQ ID NO: 11)
5′-A*A*A*G*T*C*A*C*G*A*T*T*A*C*C*T-3′
Mouse Gapmer-2
(SEQ ID NO: 12)
5′-C*C*G*T*A*T*G*C*T*G*A*A*T*C*A*A-3′
Mouse Gapmer-3
(SEQ ID NO: 13)
5′-A*T*T*T*A*A*C*T*C*A*T*T*C*G*A*T-3′

### Modification of Human Gapmer-2

DNA base: G, A, T, C
LNA base: +G, +A, +T, +C
Phosphorothioated DNA base: G*, A*, T*, C*
2′-O-methyl RNA base: mG, mA, mU, mC
Gm21
(SEQ ID NO: 14)
5′-mC*mC*mG*T*G*C*A*C*T*T*T*G*G*mU*mC*mA-3′
Gm22
(SEQ ID NO: 15)
5′-mC*mC*mG*T*G*C*A*C*T*T*T*G*mG*mU*mC*mA-3′
Gm23
(SEQ ID NO: 16)
5′-mC*mC*mG*T*G*C*A*C*T*T*T*G*G*T*mC*mA-3′
Gm24
(SEQ ID NO: 17)
5′-mC*mC*mG*mU*G*C*A*C*T*T*T*G*G*mU*mC*mA-3′
Gm25
(SEQ ID NO: 18)
5′-mC*mC*mG*mU*G*C*A*C*T*T*T*G*mG*mU*mC*mA-3′
Gm26
(SEQ ID NO: 19)
5′-mC*mC*mG*mU*G*C*A*C*T*T*T*G*G*T*mC*mA-3′
Gm27
(SEQ ID NO: 20)
5′-mC*mC*G*T*G*C*A*C*T*T*T*G*G*mU*mC*mA-3′
Gm28
(SEQ ID NO: 21)
5′-mC*mC*G*T*G*C*A*C*T*T*T*G*mG*mU*mC*mA-3′
Gm29
(SEQ ID NO: 22)
5′-mC*mC*G*T*G*C*A*C*T*T*T*G*G*T*mC*mA-3′
LNA base: (+G, +A, +T,+C)
G21:
(SEQ ID NO: 23)
5′-+C*+C*+G*T*G*C*A*C*T*T*T*G*G*+T*+C*+A-3′
G22:
(SEQ ID NO: 24)
5′-+C*+C*+G*T*G*C*A*C*T*T*T*G*+G*+T*+C*+A-3′
G23:
(SEQ ID NO: 25)
5′-+C*+C*+G*T*G*C*A*C*T*T*T*G*G*T*+C*+A-3′
G24:
(SEQ ID NO: 26)
5′-+C*+C*+G*+T*G*C*A*C*T*T*T*G*G*+T*+C*+A-3′
G25:
(SEQ ID NO: 27)
5′-+C*+C*+G*+T*G*C*A*C*T*T*T*G*+G*+T*+C*+A-3′
G26:
(SEQ ID NO: 28)
5′-+C*+C*+G*+T*G*C*A*C*T*T*T*G*G*T*+C*+A-3′
G27:
(SEQ ID NO: 29)
5′-+C*+C*G*T*G*C*A*C*T*T*T*G*G*T*+C*+A-3′
G28:
(SEQ ID NO: 30)
5′-+C*+C*G*T*G*C*A*C*T*T*T*G*G*+T*+C*+A-3′
G29:
(SEQ ID NO: 31)
5′-+C*+C*G*T*G*C*A*C*T*T*T*G*+G*+T*+C*+A-3′

### Modification of Human Gapmer-9

Gm91
(SEQ ID NO: 32)
5′-mG*mC*mG*A*T*T*G*A*T*T*C*G*A*mU*mA*mG-3′
Gm92
(SEQ ID NO: 33)
5′-mG*mC*mG*A*T*T*G*A*T*T*C*G*mA*mU*mA*mG-3′
Gm93
(SEQ ID NO: 34)
5′-mG*mC*mG*A*T*T*G*A*T*T*C*G*A*T*mA*mG-3′
Gm94
(SEQ ID NO: 35)
5′-mG*mC*mG*mA*T*T*G*A*T*T*C*G*A*mU*mA*mG-3′
Gm95
(SEQ ID NO: 36)
5′-mG*mC*mG*mA*T*T*G*A*T*T*C*G*mA*mU*mA*mG-3′
Gm96
(SEQ ID NO: 37)
5′-mG*mC*mG*mA*T*T*G*A*T*T*C*G*A*T*mA*mG-3′
Gm97
(SEQ ID NO: 38)
5′-mG*mC*G*A*T*T*G*A*T*T*C*G*A*mU*mA*mG-3′
Gm98
(SEQ ID NO: 39)
5′-mG*mC*G*A*T*T*G*A*T*T*C*G*mA*mU*mA*mG-3′
Gm99
(SEQ ID NO: 40)
5′-mG*mC*G*A*T*T*G*A*T*T*C*G*A*T*mA*mG-3′
G91
(SEQ ID NO: 41)
5′-+G*+C*+G*A*T*T*G*A*T*T*C*G*A*+T*+A*+G-3′
G92
(SEQ ID NO: 42)
5′-+G*+C*+G*A*T*T*G*A*T*T*C*G*+A*+T*+A*+G-3′
G93
(SEQ ID NO: 43)
5′-+G*+C*+G*A*T*T*G*A*T*T*C*G*A*T*+A*+G-3′
G94
(SEQ ID NO: 44)
5′-+G*+C*+G*+A*T*T*G*A*T*T*C*G*A*+T*+A*+G-3′
G95
(SEQ ID NO: 45)
5′-+G*+C*+G*+A*T*T*G*A*T*T*C*G*+A*+T*+A*+G-3′
G96
(SEQ ID NO: 46)
5′-+G*+C*+G*+A*T*T*G*A*T*T*C*G*A*T*+A*+G-3′
G97
(SEQ ID NO: 47)
5′-+G*+C*G*A*T*T*G*A*T*T*C*G*A*+T*+A*+G-3′
G98
(SEQ ID NO: 48)
5′-+G*+C*G*A*T*T*G*A*T*T*C*G*+A*+T*+A*+G-3′
G99
(SEQ ID NO: 49)
5′-+G*+C*G*A*T*T*G*A*T*T*C*G*A*T*+A*+G-3′

### Modification of Human Gapmer-10

Gm101
(SEQ ID NO: 50)
5′-mA*mA*mC*A*G*C*G*A*T*T*G*A*T*mU*mC*mG-3′
Gm102
(SEQ ID NO: 51)
5′-mA*mA*mC*A*G*C*G*A*T*T*G*A*mU*mU*mC*mG-3′
Gm103
(SEQ ID NO: 52)
5′-mA*mA*mC*A*G*C*G*A*T*T*G*A*T*T*mC*mG-3′
Gm104
(SEQ ID NO: 53)
5′-mA*mA*mC*mA*G*C*G*A*T*T*G*A*T*mU*mC*mG-3′
Gm105
(SEQ ID NO: 54)
5′-mA*mA*mC*mA*G*C*G*A*T*T*G*A*mU*mU*mC*mG-3′
Gm106
(SEQ ID NO: 55)
5′-mA*mA*mC*mA*G*C*G*A*T*T*G*A*T*T*mC*mG-3′
Gm107
(SEQ ID NO: 56)
5′-mA*mA*C*A*G*C*G*A*T*T*G*A*T*mU*mC*mG-3′
Gm108
(SEQ ID NO: 57)
5′-mA*mA*C*A*G*C*G*A*T*T*G*A*mU*mU*mC*mG-3′
Gm109
(SEQ ID NO: 58)
5′-mA*mA*C*A*G*C*G*A*T*T*G*A*T*T*mC*mG-3′
G101
(SEQ ID NO: 59)
5′-+A*+A*+C*A*G*C*G*A*T*T*G*A*T*+T*+C*+G-3′
G102
(SEQ ID NO: 60)
5′-+A*+A*+C*A*G*C*G*A*T*T*G*A*+T*+T*+C*+G-3′
G103
(SEQ ID NO: 61)
5′-+A*+A*+C*A*G*C*G*A*T*T*G*A*T*T*+C*+G-3′
G104
(SEQ ID NO: 62)
5′-+A*+A*+C*+A*G*C*G*A*T*T*G*A*T*+T*+C*+G-3′
G105
(SEQ ID NO: 63)
5′-+A*+A*+C*+A*G*C*G*A*T*T*G*A*+T*+T*+C*+G-3′
G106
(SEQ ID NO: 64)
5′-+A*+A*+C*+A*G*C*G*A*T*T*G*A*T*T*+C*+G-3′
G107
(SEQ ID NO: 65)
5′-+A*+A*C*A*G*C*G*A*T*T*G*A*T*+T*+C*+G-3′
G108
(SEQ ID NO: 66)
5′-+A*+A*C*A*G*C*G*A*T*T*G*A*+T*+T*+C*+G-3′
G109
(SEQ ID NO: 67)
5′-+A*+A*C*A*G*C*G*A*T*T*G*A*T*T*+C*+G-3′

In a gapmer design, the 5′ and 3′ most nucleosides of the gap region are DNA nucleosides, and are positioned adjacent to a sugar modified nucleoside of the 5′ (F) or 3′ (F′) region respectively. The flanks may be further defined by having at least one sugar modified nucleoside at the end most distant from the gap region, i.e., at the 5′ end of the 5′ flank and at the 3′ end of the 3′ flank.

Regions F-G-F′ form a contiguous nucleotide sequence. Antisense oligonucleotides of the present disclosure, or the contiguous nucleotide sequence thereof, may comprise a gapmer region of formula F-G-F′. The overall length of the gapmer design F-G-F′ may be, for example 12 to 32 nucleosides, such as 13 to 24, such as 14 to 22 nucleosides, such as from 14 to 17, such as 16 to 18 nucleosides.

By way of example, the gapmer oligonucleotide of the present disclosure can be represented by the following formulae: F₁₋₈-G5-16-F′₁₋₈, such as F₁₋₈-G7-16-F′2-8, with the proviso that the overall length of the gapmer regions F-G-F′ is at least 12, such as at least 14 nucleotides in length.

Regions F, G and F′ are further defined below and can be incorporated into the F-G-F′ formula.

Gapmer—Region G: Region G (gap region) of the gapmer is a region of nucleosides which enables the oligonucleotide to recruit RNaseH, such as human RNase H1, typically DNA nucleosides. RNaseH is a cellular enzyme which recognizes the duplex between DNA and RNA, and enzymatically cleaves the RNA molecule. Suitable gapmers may have a gap region (G) of at least 5 or 6 contiguous DNA nucleosides, such as 5-16 contiguous DNA nucleosides, such as 6-15 contiguous DNA nucleosides, such as 7-14 contiguous DNA nucleosides, such as 8-12 contiguous DNA nucleotides, such as 8-12 contiguous DNA nucleotides in length. The gap region G may, in some embodiments consist of 6, 7, 8, 9, 10, 11, 12, 13, 14, 15 or 16 contiguous DNA nucleosides. Cytosine (C) DNA in the gap region may in some instances be methylated, such residues are either annotated as 5-methyl-cytosine (^{me}C or with an e instead of a c). Methylation of Cytosine DNA in the gap is advantageous if cg dinucleotides are present in the gap to reduce potential toxicity, the modification does not have significant impact on efficacy of the oligonucleotides.

In some embodiments the gap region G may consist of 6, 7, 8, 9, 10, 11, 12, 13, 14, 15 or 16 contiguous phosphorothioate linked DNA nucleosides. In some embodiments, all internucleoside linkages in the gap are phosphorothioate linkages.

Whilst traditional gapmers have a DNA gap region, there are numerous examples of modified nucleosides which allow for RNaseH recruitment when they are used within the gap region. Modified nucleosides which have been reported as being capable of recruiting RNaseH when included within a gap region include, for example, alpha-L-LNA, C4′ alkylated DNA (as described in PCT/EP2009/050349 and Vester et al., Bioorg. Med. Chem. Lett. 18 (2008) 2296-2300, both incorporated herein by reference), arabinose derived nucleosides like ANA and 2′F-ANA (Mangos et al. 2003 J. AM. CHEM. SOC. 125, 654-661), UNA (unlocked nucleic acid) (as described in Fluiter et al., Mol. Biosyst., 2009, 10, 1039 incorporated herein by reference). UNA is unlocked nucleic acid, typically where the bond between C2 and C3 of the ribose has been removed, forming an unlocked “sugar” residue. The modified nucleosides used in such gapmers may be nucleosides which adopt a 2′ endo (DNA like) structure when introduced into the gap region, i.e. modifications which allow for RNaseH recruitment). In some embodiments the DNA Gap region (G) described herein may optionally contain 1 to 3 sugar modified nucleosides which adopt a 2′ endo (DNA like) structure when introduced into the gap region.

Region G—“Gap-Breaker”: Alternatively, there are numerous reports of the insertion of a modified nucleoside which confers a 3′ endo conformation into the gap region of gapmers, whilst retaining some RNaseH activity. Such gapmers with a gap region comprising one or more 3′endo modified nucleosides are referred to as “gap-breaker” or “gap-disrupted” gapmers, see for example WO2013/022984. Gap-breaker oligonucleotides retain sufficient region of DNA nucleosides within the gap region to allow for RNaseH recruitment. The ability of gapbreaker oligonucleotide design to recruit RNaseH is typically sequence or even compound specific—see Rukov et al. 2015 Nucl. Acids Res. Vol. 43 pp. 8476-8487, which discloses “gapbreaker” oligonucleotides which recruit RNaseH which in some instances provide a more specific cleavage of the target RNA. Modified nucleosides used within the gap region of gap-breaker oligonucleotides may for example be modified nucleosides which confer a 3′endo confirmation, such 2′-O-methyl (OMe) or 2′-O-MOE (MOE) nucleosides, or beta-D LNA nucleosides (the bridge between C2′ and C4′ of the ribose sugar ring of a nucleoside is in the beta conformation), such as beta-D-oxy LNA or ScET nucleosides.

As with gapmers containing region G described above, the gap region of gap-breaker or gap-disrupted gapmers, have a DNA nucleoside at the 5′ end of the gap (adjacent to the 3′ nucleoside of region F), and a DNA nucleoside at the 3′ end of the gap (adjacent to the 5′ nucleoside of region F′). Gapmers which comprise a disrupted gap typically retain a region of at least 3 or 4 contiguous DNA nucleosides at either the 5′ end or 3′ end of the gap region. Exemplary designs for gap-breaker oligonucleotides include: F₁₋₈-[D₃₋₄-E₁-D₃₋₄]-F′₁₋₈; F₁₋₈-[D₁₋₄-E₁-D₃₋₄]-F′₁₋₈: or F₁₋₈-[D₃₋₄-E₁-D₁₋₄]-F′₁₋₈ wherein region G is within the brackets [Dₙ-Eᵣ-Dₘ], D is a contiguous sequence of DNA nucleosides, E is a modified nucleoside (the gap-breaker or gap-disrupting nucleoside), and F and F′ are the flanking regions as defined herein, and with the proviso that the overall length of the gapmer regions F-G-F′ is at least 12, such as at least 14 nucleotides in length.

In some embodiments, region G of a gap disrupted gapmer comprises at least 6 DNA nucleosides, such as 6, 7, 8, 9, 10, 11, 12, 13, 14, 15 or 16 DNA nucleosides. As described above, the DNA nucleosides may be contiguous or may optionally be interspersed with one or more modified nucleosides, with the proviso that the gap region G is capable of mediating RNaseH recruitment.

Gapmer—Flanking Regions, F and F′: Region F is positioned immediately adjacent to the 5′ DNA nucleoside of region G. The 3′ most nucleoside of region F is a sugar modified nucleoside, such as a high affinity sugar modified nucleoside, for example a 2′ substituted nucleoside, such as a MOE nucleoside, or an LNA nucleoside.

Region F′ is positioned immediately adjacent to the 3′ DNA nucleoside of region G. The 5′ most nucleoside of region F′ is a sugar modified nucleoside, such as a high affinity sugar modified nucleoside, for example a 2′ substituted nucleoside, such as a MOE nucleoside, or an LNA nucleoside.

Region F is 1-8 contiguous nucleotides in length, such as 2-6, such as 3-4 contiguous nucleotides in length. Advantageously the 5′ most nucleoside of region F is a sugar modified nucleoside. In some embodiments the two 5′ most nucleoside of region F are sugar modified nucleoside. In some embodiments the 5′ most nucleoside of region F is an LNA nucleoside. In some embodiments the two 5′ most nucleoside of region F are LNA nucleosides. In some embodiments the two 5′ most nucleoside of region F are 2′ substituted nucleoside nucleosides, such as two 3′ MOE nucleosides. In some embodiments the 5′ most nucleoside of region F is a 2′ substituted nucleoside, such as a MOE nucleoside.

Region F′ is 2-8 contiguous nucleotides in length, such as 3-6, such as 4-5 contiguous nucleotides in length. Advantageously, embodiments the 3′ most nucleoside of region F′ is a sugar modified nucleoside. In some embodiments the two 3′ most nucleoside of region F′ are sugar modified nucleoside. In some embodiments the two 3′ most nucleoside of region F′ are LNA nucleosides. In some embodiments the 3′ most nucleoside of region F′ is an LNA nucleoside. In some embodiments the two 3′ most nucleoside of region F′ are 2′ substituted nucleoside nucleosides, such as two 3′ MOE nucleosides. In some embodiments the 3′ most nucleoside of region F′ is a 2′ substituted nucleoside, such as a MOE nucleoside.

It should be noted that when the length of region F or F′ is one, it is advantageously an LNA nucleoside. In some embodiments, region F and F′independently consists of or comprises a contiguous sequence of sugar modified nucleosides. In some embodiments, the sugar modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units. In some embodiments, region F and F′ independently comprises both LNA and a 2′ substituted modified nucleosides (mixed wing design).

In some embodiments, region F and F′ consists of only one type of sugar modified nucleosides, such as only MOE or only beta-D-oxy LNA or only ScET. Such designs are also termed uniform flanks or uniform gapmer design.

In some embodiments, all the nucleosides of region F or F′, or F and F′ are LNA nucleosides, such as independently selected from beta-D-oxy LNA, ENA or ScET nucleosides. In some embodiments region F consists of 1-5, such as 2-4, such as 3-4 such as 1, 2, 3, 4 or 5 contiguous LNA nucleosides. In some embodiments, all the nucleosides of region F and F′ are beta-D-oxy LNA nucleosides.

In some embodiments, all the nucleosides of region F or F′, or F and F′ are 2′ substituted nucleosides, such as OMe or MOE nucleosides. In some embodiments region F consists of 1, 2, 3, 4, 5, 6, 7, or 8 contiguous OMe or MOE nucleosides. In some embodiments only one of the flanking regions can consist of 2′ substituted nucleosides, such as OMe or MOE nucleosides. In some embodiments it is the 5′ (F) flanking region that consists 2′ substituted nucleosides, such as OMe or MOE nucleosides whereas the 3′ (F′) flanking region comprises at least one LNA nucleoside, such as beta-D-oxy LNA nucleosides or cET nucleosides. In some embodiments it is the 3′ (F′) flanking region that consists 2′ substituted nucleosides, such as OMe or MOE nucleosides whereas the 5′ (F) flanking region comprises at least one LNA nucleoside, such as beta-D-oxy LNA nucleosides or cET nucleosides.

In some embodiments, all the modified nucleosides of region F and F′ are LNA nucleosides, such as independently selected from beta-D-oxy LNA, ENA or ScET nucleosides, wherein region F or F′, or F and F′ may optionally comprise DNA nucleosides (an alternating flank, see definition of these for more details). In some embodiments, all the modified nucleosides of region F and F′ are beta-D-oxy LNA nucleosides, wherein region F or F′, or F and F′ may optionally comprise DNA nucleosides (an alternating flank, see definition of these for more details).

In some embodiments the 5′ most and the 3′ most nucleosides of region F and F′ are LNA nucleosides, such as beta-D-oxy LNA nucleosides or ScET nucleosides.

In some embodiments, the internucleoside linkage between region F and region G is a phosphorothioate internucleoside linkage. In some embodiments, the internucleoside linkage between region F′ and region G is a phosphorothioate internucleoside linkage. In some embodiments, the internucleoside linkages between the nucleosides of region F or F′, F and F′ are phosphorothioate internucleoside linkages.

Further gapmer designs are disclosed in WO 2004/046160, WO 2007/146511 and WO 2008/113832, hereby incorporated by reference.

LNA Gapmer: An LNA gapmer is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In some embodiments the LNA gapmer is of formula: [LNA]₁₋₅-[region G]-[LNA]₁₋₅, wherein region G is as defined in the Gapmer region G definition.

MOE Gapmers: A MOE gapmers is a gapmer wherein regions F and F′ consist of MOE nucleosides. In some embodiments the MOE gapmer is of design [MOE]₁₋₈-[Region G]-[MOE]₁₋₈, such as [MOE]₂₋₇-[Region G]₅₋₁₆-[MOE]₂₋₇, such as [MOE]₃₋₆-[Region G]-[MOE]₃₋₆, wherein region G is as defined in the Gapmer definition. MOE gapmers with a 5-10-5 design (MOE-DNA-MOE) have been widely used in the art.

Mixed Wing Gapmer: A mixed wing gapmer is an LNA gapmer wherein one or both of region F and F′ comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units, such as a MOE nucleosides. In some embodiments wherein at least one of region F and F′, or both region F and F′ comprise at least one LNA nucleoside, the remaining nucleosides of region F and F′ are independently selected from the group consisting of MOE and LNA. In some embodiments wherein at least one of region F and F′, or both region F and F′ comprise at least two LNA nucleosides, the remaining nucleosides of region F and F′ are independently selected from the group consisting of MOE and LNA. In some mixed wing embodiments, one or both of region F and F′ may further comprise one or more DNA nucleosides. Mixed wing gapmer designs are disclosed in WO 2008/049085 and WO 2012/109395, both of which are hereby incorporated by reference.

Alternating Flank Gapmers: Flanking regions may comprise both LNA and DNA nucleoside and are referred to as “alternating flanks” as they comprise an alternating motif of LNA-DNA-LNA nucleosides. Gapmers comprising such alternating flanks are referred to as “alternating flank gapmers”. “Alternative flank gapmers” are thus LNA gapmer oligonucleotides where at least one of the flanks (F or F′) comprises DNA in addition to the LNA nucleoside(s). In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F and/or F′ region are LNA nucleosides. Alternating flank LNA gapmers are disclosed in WO 2016/127002. An alternating flank region may comprise up to 3 contiguous DNA nucleosides, such as 1 to 2 or 1 or 2 or 3 contiguous DNA nucleosides. The alternating flak can be annotated as a series of integers, representing a number of LNA nucleosides (L) followed by a number of DNA nucleosides (D), for example [L]₁₋₃-[D]₁₋₄-[L]₁₋₃ or [L]₁₋₂-[D]₁₋₂-[L]₁₋₂-[D]₁₋₂-[L]₁₋₂. In oligonucleotide designs these will often be represented as numbers such that 2-2-1 represents 5′ [L]2-[D]2-[L] 3′, and 1-1-1-1-1 represents 5′ [L]-[D]-[L]-[D]-[L] 3′. The length of the flank (region F and F′) in oligonucleotides with alternating flanks may independently be 3 to 10 nucleosides, such as 4 to 8, such as 5 to 6 nucleosides, such as 4, 5, 6 or 7 modified nucleosides. In some embodiments only one of the flanks in the gapmer oligonucleotide is alternating while the other is constituted of LNA nucleotides. It may be advantageous to have at least two LNA nucleosides at the 3′ end of the 3′ flank (F′), to confer additional exonuclease resistance.

Region D′ or D″ in an Oligonucleotide: The oligonucleotide of the disclosure may in some embodiments comprise or consist of the contiguous nucleotide sequence of the oligonucleotide which is complementary to the target nucleic acid, such as the gapmer F-G-F′, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein.

The addition of region D′ or D″ may be used for the purpose of joining the contiguous nucleotide sequence, such as the gapmer, to a conjugate moiety or another functional group. When used for joining the contiguous nucleotide sequence with a conjugate moiety is can serve as a biocleavable linker. Alternatively, it may be used to provide exonuclease protection or for ease of synthesis or manufacture.

### D. Modified Nucleotides

In certain embodiments, compounds of the disclosure comprise one or more modified nucleosides comprising a modified sugar moiety. Such compounds comprising one or more sugar-modified nucleosides may have desirable properties, such as enhanced nuclease stability or increased binding affinity with a target nucleic acid relative to an oligonucleotide comprising only nucleosides comprising naturally occurring sugar moieties. In certain embodiments, modified sugar moieties are substituted sugar moieties. In certain embodiments, modified sugar moieties are sugar surrogates. Such sugar surrogates may comprise one or more substitutions corresponding to those of substituted sugar moieties.

In certain embodiments, modified sugar moieties are substituted sugar moieties comprising one or more non-bridging sugar substituent, including but not limited to substituents at the 2′ and/or 5′ positions. Examples of sugar substituents suitable for the 2′-position, include, but are not limited to: 2′-F, 2′-OCH₃ (“OMe” or “O-methyl”), and 2′-O(CH₂)₂OCH₃ (“MOE”). In certain embodiments, sugar substituents at the 2′ position is selected from allyl, amino, azido, thio, O-allyl, O—C₁-C₁₀ alkyl, O—C₁-C₁₀ substituted alkyl; OCF₃, O(CH₂)₂SCH₃, O(CH₂)₂—O—N(Rm)(Rn), and O—CH₂—C(═O)—N(Rm)(Rn), where each Rm and Rn is, independently, H or substituted or unsubstituted C₁-C₁₀ alkyl. Examples of sugar substituents at the 5′-position, include, but are not limited to: 5′-methyl (R or S); 5′-vinyl, and 5′-methoxy. In particular embodiments, nucleic acids may be modified with a 5′-vinylphosphonate to increase the stability of the nucleic acid and improve tissue accumulation. In certain embodiments, substituted sugars comprise more than one non-bridging sugar substituent, for example, T-F-5′-methyl sugar moieties (see, e.g., PCT Application WO 2008/101157, for additional 5′,2′-bis substituted sugar moieties and nucleosides).

Nucleosides comprising 2′-substituted sugar moieties are referred to as 2′-substituted nucleosides. In certain embodiments, a 2′-substituted nucleoside comprises a 2′-substituent group selected from halo, allyl, amino, azido, SH, CN, OCN, CF₃, OCF₃, O, S, or N(Rₘ)-alkyl; O, S, or N(Rₘ)-alkenyl; O, S or N(Rₘ)-alkynyl; O-alkylenyl-O-alkyl, alkynyl, alkaryl, aralkyl, O-alkaryl, O-aralkyl, O(CH₂)₂SCH₃, O(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In certain embodiments, a 2′-substituted nucleoside comprises a 2′-substituent group selected from F, NH₂, N₃, OCF₃, O—CH₃, O(CH₂)₃NH₂, CH₂—CH═CH₂, O—CH₂—CH═CH₂, OCH₂CH₂OCH₃, O(CH₂)₂SCH₃, O—(CH₂)₂—O—N(Rₘ)(Rₙ), O(CH₂)₂O(CH₂)₂N(CH₃)₂, and N-substituted acetamide (O—CH₂—C(═O)—N(Rₘ)(Rₙ) where each Rₘ and Rₙ is, independently, H, an amino protecting group or substituted or unsubstituted C₁-C₁₀ alkyl.

In certain embodiments, a 2′-substituted nucleoside comprises a sugar moiety comprising a 2′-substituent group selected from F, OCF₃, O—CH₃, OCH₂CH₂OCH₃, O(CH₂)₂SCH₃, O(CH₂)₂—O—N(CH₃)₂, —O(CH₂)₂O(CH₂)₂N(CH₃)₂, and O—CH₂—C(═O)—N(H)CH₃.

In certain embodiments, a 2′-substituted nucleoside comprises a sugar moiety comprising a 2′-substituent group selected from F, O—CH₃, and OCH₂CH₂OCH₃.

In certain embodiments, modified sugar moieties are sugar surrogates. In certain such embodiments, the oxygen atom of the naturally occurring sugar is substituted, e.g., with a sulfur, carbon or nitrogen atom. In certain such embodiments, such modified sugar moiety also comprises bridging and/or non-bridging substituents as described above. For example, certain sugar surrogates comprise a 4′-sulfur atom and a substitution at the 2′-position (see, e.g., published U.S. Patent Publication No. 20050130923, published on Jun. 16, 2005) and/or the 5′ position. By way of additional example, carbocyclic bicyclic nucleosides having a 4′-2′ bridge have been described (see, e.g., Freier et al., Nucleic Acids Research, 1997, 25 (22), 4429-4443 and Albaek et al., J. Org. Chem., 2006, 71, 7731-7740).

In certain embodiments, sugar surrogates comprise rings having other than 5-atoms. For example, in certain embodiments, a sugar surrogate comprises a six-membered tetrahydropyran. Such tetrahydropyrans may be further modified or substituted. Nucleosides comprising such modified tetrahydropyrans include, but are not limited to, hexitol nucleic acid (HNA), altritol nucleic acid (ANA), mannitol nucleic acid (MNA) (see Leumann, C J. Bioorg. & Med. Chem. (2002) 10:841-854), and fluoro HNA (F-HNA).

In certain embodiments, the present disclosure provides oligonucleotides comprising modified nucleosides. Those modified nucleotides may include modified sugars, modified nucleobases, and/or modified linkages. The specific modifications are selected such that the resulting oligonucleotides possess desirable characteristics. In certain embodiments, oligonucleotides comprise one or more RNA-like nucleosides. In certain embodiments, oligonucleotides comprise one or more DNA-like nucleotides.

In certain embodiments, nucleosides of the present disclosure comprise one or more unmodified nucleobases. In certain embodiments, nucleosides of the present disclosure comprise one or more modified nucleobases.

In certain embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and O-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine ([5,4-b][1,4]benzoxazin-2 (3H)-one), phenothiazine cytidine (1H-pyrimido[5,4-b][1,4]benzothiazin-2 (3H)-one), G-clamps such as a substituted phenoxazine cytidine (e.g., 9-(2-aminoethoxy)-H-pyrimido[5,4-13][1,4]benzoxazin-2 (3H)-one), carbazole cytidine (²H-pyrimido[4,5-b]indol-2-one), pyridoindole cytidine (H-pyrido[3′,2′: 4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; those disclosed by Englisch et al., Angewandte Chemie, International Edition, 1991, 30, 613; and those disclosed by Sanghvi, Y. S., Chapter 15, Antisense Research and Applications, Crooke, S. T. and Lebleu, B., Eds., CRC Press, 1993, 273-288.

Representative United States Patents that teach the preparation of certain of the above noted modified nucleobases as well as other modified nucleobases include without limitation, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096, each of which is herein incorporated by reference in its entirety.

In certain embodiments, the present disclosure provides oligonucleotides comprising linked nucleosides. In such embodiments, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, methylenephosphonates, vinylphosphonates, phosphonoacetates, thiophosphonoacetates, phosphoramidates, and phosphorothioates (P═S). Representative non-phosphorus containing internucleoside linking groups include, but are not limited to, amide, triazole, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotide. In certain embodiments, internucleoside linkages having a chiral atom can be prepared as a racemic mixture, or as separate enantiomers. Representative chiral linkages include, but are not limited to, alkylphosphonates and phosphorothioates. Methods of preparation of phosphorous-containing and non-phosphorous-containing internucleoside linkages are well known to those skilled in the art.

The oligonucleotides described herein contain one or more asymmetric centers and thus give rise to enantiomers, diastereomers, and other stereoisomeric configurations that may be defined, in terms of absolute stereochemistry, as (R) or(S), a or β such as for sugar anomers, or as (D) or (L) such as for amino acids, etc. Included in the antisense compounds provided herein are all such possible isomers, as well as their racemic and optically pure forms.

Neutral internucleoside linkages include without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), other amides, formacetal (3′-O—CH₂—O-5′), and thioformacetal (3′-S—CH₂—O-5′), and triazole linkages. Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

Additional modifications may also be made at other positions on the oligonucleotide, particularly the 3′ position of the sugar on the 3′ terminal nucleotide and the 5′ position of 5′ terminal nucleotide. For example, one additional modification of the ligand conjugated oligonucleotides of the present disclosure involves chemically linking to the oligonucleotide one or more additional non-ligand moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA, 1989, 86, 6553), cholic acid (Manoharan et al., Bioorg. Med. Chem. Lett., 1994, 4, 1053), a thioether, e.g., hexyl-5-tritylthiol (Manoharan et al., Ann. N.Y. Acad. Sci., 1992, 660, 306; Manoharan et al., Bioorg. Med. Chem. Let., 1993, 3, 2765), a thiocholesterol (Oberhauser et al., Nucl. Acids Res., 1992, 20, 533), an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651; Shea et al., Nucl. Acids Res., 1990, 18, 3777), a polyamine or a polyethylene glycol chain (Manoharan et al., Nucleosides & Nucleotides, 1995, 14, 969), or adamantane acetic acid (Manoharan et al., Tetrahedron Lett., 1995, 36, 3651), a palmityl moiety (Mishra et al., Biochim. Biophys. Acta, 1995, 1264, 229), or an octadecylamine or hexylamino-carbonyl-oxycholesterol moiety (Crooke et al., J. Pharmacol. Exp. Ther., 1996, 277, 923).

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned, and each of which is herein incorporated by reference.

### E. Oligonucleotide Delivery

There are a number of ways in which oligonucleotides may introduced into cells. In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the delivery of oligonucleotides are contemplated by the present disclosure. These include calcium phosphate precipitation, DEAE-dextran, electroporation, direct microinjection, DNA-loaded liposomes and lipofectamine-DNA complexes, cell sonication, gene bombardment using high velocity microprojectiles, and receptor-mediated transfection. Some of these techniques may be successfully adapted for in vivo or ex vivo use.

In a further embodiment of the disclosure, the oligonucleotides may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated are lipofectamine-DNA complexes.

Liposome-mediated nucleic acid delivery and expression of foreign DNA in vitro has been very successful. The feasibility of liposome-mediated delivery and expression of foreign DNA in cultured chick embryo, HeLa and hepatoma cells has been demonstrated, and successful liposome-mediated gene transfer in rats after intravenous injection has been achieved. A reagent known as Lipofectamine 2000™ is widely used and commercially available.

In certain embodiments of the disclosure, the liposome may be complexed with a hemagglutinating virus (HVJ). This has been shown to facilitate fusion with the cell membrane and promote cell entry of liposome-encapsulated DNA. In other embodiments, the liposome may be complexed or employed in conjunction with nuclear non-histone chromosomal proteins (HMG-1). In yet further embodiments, the liposome may be complexed or employed in conjunction with both HVJ and HMG-1. In that such expression constructs have been successfully employed in transfer and expression of nucleic acid in vitro and in vivo, then they are applicable for the present disclosure. Where a bacterial promoter is employed in the DNA construct, it also will be desirable to include within the liposome an appropriate bacterial polymerase.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin. A synthetic neoglycoprotein, which recognizes the same receptor as ASOR, has been used as a gene delivery vehicle and epidermal growth factor (EGF) has also been used to deliver genes to squamous carcinoma cells.

### 1. Nanoparticles

In some aspects, the oligonucleotides are delivered by nanoparticles. Particles having an average diameter on the nanometer scale (e.g., from about 0.1 nm to about 500 nm) are referred to as “nanoparticles.”

As used herein, the term “polymer” generally includes, but is not limited to, homopolymers; copolymers, such as, for example, block, graft, random and alternating copolymers; and terpolymers; and blends and modifications thereof. Furthermore, unless otherwise specifically limited, the term “polymer” shall include all possible geometrical configurations of the material. These configurations include, but are not limited to isotactic, syndiotactic, and random symmetries.

The nanoparticles may be dual-targeted and dual-responsive nanoparticles (DTDRN) as described in U.S. Patent Application No. US20160346208A1; incorporated herein by reference in its entirety.

In particular aspects, the present nanoparticles are suitable for delivery of materials across the blood brain barrier. More specifically, the nanoparticles include a biocompatible hydrophilic polymer and two (or more) types of surface ligands that can encourage transport across the blood brain barrier and then be detached from the nanoparticles by acidic pH and/or high redox potential as may be found in the lysosome or following crossing of the blood brain barrier so as to release the payload carried by the nanoparticles. The nanoparticles can also include a biologically active compound such as a drug, e.g., encapsulated in the nanoparticle or attached to the surface of the nanoparticle, for delivery following crossing of the blood brain barrier by the nanoparticles.

Also disclosed are methods of forming the nanoparticles and methods of using the nanoparticles. For instance, the dual responsive nanoparticles can be formed by conjugation of a hydrophilic biocompatible polymer with the two different ligands by formation of acid-sensitive and/or redox potential-sensitive bonds and by forming the polymer as a nanoparticle, for instance by crosslinking the hydrophilic biocompatible polymer. The dual responsive nanoparticles can also be loaded with a biologically active agent for delivery across the blood brain barrier either during or following particle formation.

The nanoparticles can be prepared from polymeric materials that can be biocompatible, provide long circulation life in a body, and that can be successfully ligated to at least two different ligands via an acidic responsive and/or redox potential-responsive bond formation. For instance, one or both of the functional ligands can be directly or indirectly bonded to the nanoparticle via an acid-sensitive bond such as, without limitation an ester bond, a hydrazone bond, or a cis-aconityl bond. Alternatively, one or both of the functional ligands can be directly or indirectly bonded to the nanoparticle via a redox potential-sensitive bond such as, without limitation, a disulfide bond. Of course, a ligating bond can be both acidic-sensitive and redox potential-sensitive.

As utilized herein, an acidic-sensitive bond can generally refer to a bond that will degrade or otherwise break in an environment of about pH 6.8 or less, for instance about pH 4 to about pH 6.8, and will be more stable in an environment at higher pH (e.g., about 7 or higher). A redox potential-sensitive bond can generally refer to a bond that will degrade in an environment having a redox potential equal to that of a glutathione concentration of from about 0.1 mM to about 10 mM).

Materials that can be ligated to the nanoparticles can include any material that exhibits blood brain barrier transport capabilities. As utilized herein, the term “blood brain barrier transporter” refers to a material that can naturally pass the blood brain barrier. Moreover, a blood brain barrier transporter can encompass a complete transporter as found in nature or a portion or fragment of the natural compound, e.g., only that portion of a transporter that binds a barrier protein as well as synthetic compounds that function as a blood brain barrier transporter. By way of example, and without limitation, suitable transporter functional ligands can include scopine, glutathione, transferrin, melanotransferrin, adenosine, insulin, low-density lipoprotein, leptin, thiamine, rabies virus glycoprotein, TAT peptide, encephalin, angiopep-2, diphtheria toxin, and tetanus toxin. In general, any combination of two (or more) of such transport-capable compounds ligated to a biocompatible nanoparticle as described is encompassed herein.

In one particular embodiment, the Dual Targeted and Dual Responsive Nanoparticles (DTDRN) can be functionalized to include scopine in conjunction with glutathione. Scopine is a tropane alkaloid found in a variety of plants including mandragora root, senecio mikanoides (Delairea odorata), scopolia carniolica, and scopolia lurida. Scopine can be prepared by the hydrolysis of scopolamine. Scopine HCI salt is the metabolite of anisodine, which is an α1-adrenergic receptor agonist and has shown activity as a brain targeting moiety (see, e.g., Wang, et al., Bioconjugate Chem., 2014, 25 (11), pp 2046-2054).

Glutathione (GSH) is an endogenous antioxidant. If its concentration in serum is insufficient, some nervous diseases, such as chronic fatigue syndrome, may occur. Research has found that a Na-dependent GSH transporter located on the luminal side of the blood brain barrier manages GSH uptake and a Na-independent GSH transporter located on the luminal side of the blood brain barrier manages efflux of GSH. Through conjugation of both scopine and glutathione to a nanoparticle delivery system, improved delivery of biologically active compounds across the blood brain barrier can be achieved.

The hydrophilic component of the polymer can be based upon any biocompatible polymer or oligomer capable of reacting with the desired pyridine-2-thiol monomers. By way of example and without limitation, the hydrophilic component can include one or more of polyethylene glycol, poly(N-isopropylacrylamide) (polyNIPAAm), poly(N-(2-hydroxypropyl) methacrylamide) (polyHPMA), poly(acrylic acid) (PAAc), poly(DL-lactic acid-co-glycolic acid) (PLGA), poly(L-histidine), etc.

In one representative embodiment, the nanoparticles can be prepared by initially forming a copolymer according to reaction of Poly[(2-(pyridin-2-yldisulfanyl)ethyl acrylate-co-[poly(ethylene glycol) (PDA-PEG) followed by functionalization (e.g., amine, acid, imide etc.) via, e.g., thiol-disulfide exchange reaction. Following, the functionalized polymer can be conjugated with one or both of the transporter ligands, e.g., scopine and glutathione, via the formation of bonds that are acid-sensitive and/or redox potential-sensitive bonds. For example, scopine can be conjugated to an acid-functionalized polymer to form an ester link between the polymer and the scopine ligand, while glutathione can be conjugated to a maleimide-functionalized polymer via a sulfur linkage and then conjugated to a nanoparticle component via carbodiimide chemistry to form an ester link between the nanoparticle component and the glutathione ligand. Therefore, both scopine and glutathione are indirectly conjugated to the nanoparticle through PDA segments which contain both esters bonds and disulfide bonds.

The particle form of the delivery system can be provided via crosslinking of the polymeric component. For instance, a PDA-PEG polymer can be subjected to disulfide bond cleavage followed by oxidation to crosslink the polymers and form a nanoparticle. In addition, the functional ligands can be conjugated to the nanoparticles either prior to or following crosslinking and particulate formation. For instance, one or both of the functional ligands can be surface conjugated to the nanoparticles following crosslinking and particle formation to form the delivery system that can facilitate nanoparticle penetrate through the blood brain barrier.

The dual targeted nanoparticles thus formed are labile in environments with low pH and/or high redox potential such as the brain (e.g., pH 6.5 and GSH 2.7 mM), which makes the carriers ideal for brain targeted delivery. Due to the unique dual targeted and dual responsive properties provided in certain embodiments, the disclosed systems can serve as a one-way shuttle for the delivery of drugs specifically to the brain.

The nanoparticle delivery system can be responsive to acidic pH and/or high glutathione environment, and the pH in the brain tissue is low and the GSH level is high, which makes the nanoparticle delivery system an ideal tool for brain targeted delivery. The payload (i.e., the drug compound to be delivered to the brain) can be encapsulated into the nanoparticle by hydrophobic interaction or chemically conjugated to the surface through, e.g., —S—S—, —CONH—, or —COO— bonds. Examples of biologically active compounds as may be delivered by use of a system can include, without limitation, n-acetyl cysteine, pyrrolidine dithiocarbamate, disulfiram, diethyldithiocarbamate, tangeritin, resveratrol, indometacin, paclitaxel, doxorubicin, temozolomide, curcumin, carboplatin, carmustine, cisplatin, cyclophosphamide, etoposide, irinotecan, lomustine, methotrexate, procarbazine, vincristine, sulindac, etc., as well as combinations of active agent.

### 2. Cholesterol Heterodimer

In some aspects, the gapmer may be delivered as a cholesterol RNA: DNA heteroduplex (e.g., described in U.S. Patent Publication No. US20160145614; incorporated herein by reference in its entirety). A double-stranded nucleic acid agent that can deliver a therapeutic oligonucleotide is provided which comprises a first and a second strand, wherein the therapeutic oligonucleotide (i.e., the present Fli-1 gapmer) is a portion of the first strand. The double-stranded nucleic acid agent may comprise a first strand comprising a first RNA region, and a second strand comprising a first DNA region, wherein said first RNA region and said first DNA region are hybridized as a RNA/DNA heteroduplex. In some aspects, the first strand comprises the therapeutic oligonucleotide region and a first complementary region. The first complementary region is complementary to a portion or the whole of the second strand. The second strand comprises a second complementary region. The second complementary region is complementary to a portion or the whole of the first complementary region, and optionally may be complementary to a portion of the therapeutic region.

In some embodiments, the first complementary region is designed to be resistant to nuclease degradation, or more generally, metabolic degradation, when put into a biological sample (cell, tissue, animal, human, etc.). For example, the first complementary region may comprise bases (modified nucleotides or nucleotide analogues) that are more resistant to nuclease cleavage than natural bases, or it may comprise PNA. When the first complementary region is designed to be resistant to nuclease degradation, this region will generally remain connected to the therapeutic oligonucleotide in the methods disclosed herein. In this case, when reference is made to the release of the therapeutic oligonucleotide, it may be properly understood to mean the release of the first strand (the therapeutic oligonucleotide connected to the first complementary region).

In other embodiments, the first complementary region is designed to be susceptible to cleavage by RNase H when the first strand is hybridized to the second strand. Thus, at least a portion of the first complementary region and the second complementary region are designed to be recognized by RNase H when the two strands are annealed as a duplex. Generally, for this purpose the first strand comprises RNA nucleotides and the second strand comprises DNA nucleotides in order to form a heteroduplex. In some embodiments, the first complementary region comprises 2, 3, 4, or 5 or more consecutive natural RNA bases, which may optionally be flanked on one or both sides by modified RNA nucleotides. The portion of the second complementary region that is complementary to such first complementary region may comprise natural DNA, modified DNA, DNA analogues, or other such bases that promote the recognition of the heteroduplex structure and cleavage of the first strand.

In some embodiments, the therapeutic oligonucleotide region is designed to be resistant to nuclease degradation, or more generally, metabolic degradation, when put into a biological sample (cell, tissue, animal, human, etc.). For example, the therapeutic oligonucleotide region may comprise bases (modified nucleotides or nucleotide analogues) that are more resistant to nuclease cleavage than natural bases, or it may comprise PNA. In particular, the therapeutic oligonucleotide region is generally designed to have at least 1, 2, 3, or at least 4 bases at the 3′ and the 5′ terminal portion of the region that are more resistant to cleavage or degradation than natural bases. As noted above, some of these bases at one of the end portions may also be part of the first complementary region.

When the first complementary region contains some bases that are susceptible to cleavage or degradation, such as by RNase H, and a cleavage reaction occurs, the portion of the first strand that contains the therapeutic oligonucleotide region may still yet contain a part of the first complementary region. Some of these bases that were part of the first complementary region may be susceptible to further cleavage or degradation reactions. For example, such bases may be subject to cleavage by exonucleases. Even if such bases may be present they may be removed in the sample environment, but the presence of bases at the terminal portion of the therapeutic nucleotide region that are more resistant to cleavage or degradation than natural bases are expected to prevent the cleavage or degradation of the therapeutic oligonucleotide, such as by further action by exonucleases.

In still other embodiments, additional nucleotides or analogues may be included added at the 5′ end, at the 3′ end, or at both ends of either the first or second strands, and/or at either or both ends of the therapeutic oligonucleotide region, that display nuclease resistance and low binding affinity for proteins and protein-like cellular components.

The first and second complementary regions may comprise natural and/or non-natural nucleotides. The type of nucleotides selected for the complementary regions largely determines the type of mechanism(s) by which the therapeutic oligonucleotide may be released. For example, including RNA and/or RNA-like nucleotides in the first strand and DNA and/or DNA like nucleotides in the second strand leads to the formation of DNA/RNA heteroduplex structures that can be recognized by RNase H. Thus, RNase H-dependent mechanism of action is possible in this case, in which case the first strand can be cleaved by RNase H.

The RNA-DNA may further comprise a lipid selected from cholesterol, a tocopherol, a tocotrienol, a fatty acid, a lipid-soluble vitamin, a glycolipid, and a glyceride. Examples of such a lipid include lipids such as cholesterol and fatty acids (for example, vitamin E (tocopherols, tocotrienols), vitamin A, and vitamin D); lipid-soluble vitamins such as vitamin K (for example, acylcarnitine); intermediate metabolites such as acyl-CoA; glycolipids, glycerides, and derivatives thereof. The lipid may be joined to the 3′-terminal nucleotide or the 5′-terminal nucleotide of the second nucleic acid strand, or to the 3′-terminal nucleotide or the 5′-terminal nucleotide of the first nucleic acid strand.

In an exemplary embodiment, the cholesterol of the RNA: DNA heteroduplex is cholesterol TEG (triethylene glycol spacer) with the below structure:

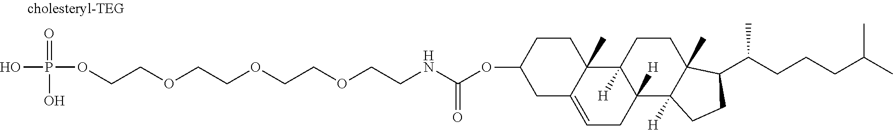

The DNA may have the sequence:

(SEQ ID NO: 23)
5′+C*+C*+G*T*G*C*A*C*T*T*T*G*G*+T*+C*+A-3′.

The cholesterol-RNA may comprise the below sequence, wherein a phosphorothioated RNA base is G*, U*, T*, or C*, a 2′-O-methyl RNA base is mG, mA, mU, or mC, and a LNA base is +G, +A, +T, or +C.

(SEQ ID NO: 68)
Cholesterol-5′-mU*mG*mA*CCAAAGUGCA*mC*mG*mG-3′

Thus, the RNA: DNA heteroduplex may comprise the sequence:

(SEQ ID NO: 69)
5′-+C*+C*+G*T*G*C*A*C*T*T*T*G*G*+T*+C*+A-3′
3′-mG*mG*mC*ACGUGAAACC*mA*mG*mU-5′-Cholesterol

### 3. Extracellular Vesicles

In some aspects, the present gapmers are delivered by extracellular vesicles. The cells for production of the EVs may be mesenchymal stem cells (MSCs) or endothelial progenitor cells (EPCs). The MSCs or EPCs may be human, which may be autologous or allogeneic.

The present gapmers may be introduced into the EVs using methods established in the art for introduction of cargo into cells. Thus, gapmers may be introduced into EVs, for example, using electroporation. Transfection using cationic lipid-based transfection reagents may also be used to introduce cargo into EVs. Examples of suitable transfection reagents include, but are not limited to, Lipofectamine MessengerMAX™ Transfection Reagent, Lipofectamine RNAiMAX Transfection Reagent, Lipofectamine 3000 Transfection Reagent, or Lipofectamine LTX Reagent with PLUS™ Reagent. For cargo loading, a suitable amount of transfection reagent is used and may vary with the reagent, the sample and the cargo. Other methods may also be utilized to introduce cargo into EVs, for example, the use of cell-penetrating peptides for protein introduction.

In an exemplary method, the present gapmers (e.g., 25 uM in 10 ul) may be mixed with EVs (e.g., 10 ul) derived from MSCs or EPCs. The gapmer can loaded into EVs by electroporation, freeze-thaw cycles, incubation at room temperature, sonication or extrusion. Droplet size (e.g., 1 ul) of EVs loaded with gapmers may be delivered intranasally, such as every 2 minutes for a total of 15 ul of EVs.

“Extracellular vesicles” and “EVs” are cell-derived and cell-secreted microvesicles which, as a class, include exosomes, exosome-like vesicles, ectosomes (which result from budding of vesicles directly from the plasma membrane), microparticles, microvesicles, shedding microvesicles (SMVs), nanoparticles and even (large) apoptotic blebs or bodies (resulting from cell death) or membrane particles.

As used herein, the terms “microvesicles” and “MVs” typically mean larger extracellular membrane vesicles or structures surrounded by a phospholipid bilayer that are about 100 nm to about 1,000 nm in diameter, or about 100 nm to about 400 nm in blood plasma. Microvesicles/MVs are formed by regulated release by budding or blebbing of the plasma membrane.

Within the class of extracellular vesicles, important components are “exosomes” themselves, which are preferably described as between about 40-120 nm, such as 50-100 nm in diameter and being membranous vesicles, i.e., vesicles surrounded by a phospholipid bilayer, of endocytic origin, which result from exocytic fusion, or “exocytosis” of multivesicular bodies (MVBs). Exosomes may be isolated from any suitable biological sample from a mammal, including but not limited to, whole blood, serum, plasma, urine, saliva, breast milk, cerebrospinal fluid, amniotic fluid, ascitic fluid, bone marrow and cultured mammalian cells (e.g. immature dendritic cells (wild-type or immortalized), induced and non-induced pluripotent stem cells, fibroblasts, platelets, immune cells, reticulocytes, tumor cells, mesenchymal stem cells, satellite cells, hematopoietic stem cells, pancreatic stem cells, white and beige pre-adipocytes and the like).

The term “mesenchymal stem cell” or “MSC”, as used herein, refers to a multipotent somatic stem cell derived from mesoderm, having self-regenerating and differentiating capacity to produce progeny cells with a large phenotypic variety, including connective tissues, stroma of bone marrow, adipocytes, dermis and muscle, among others. MSCs generally have a cell marker expression profile characterized in that they are negative for the markers CD19, CD45, CD14 and HLA-DR, and positive for the markers CD105, CD106, CD90 and CD73. MSCs may be isolated from any type of tissue. Generally, MSCs may be isolated from bone marrow, adipose tissue, umbilical cord, or peripheral blood.

The MSCs from which the EVs are derived can be autologous, allogeneic or xenogeneic. As used herein, the term “autologous” means that the donor of the MSCs and the recipient of the EVs derived from said MSCs are the same subject. The term “allogeneic” means that the donor of the MSCs and the recipient of EVs derived from said MSCs are different subjects. The term “xenogeneic” means that the donor of the MSCs and the recipient of the EVs derived from said MSCs are subjects of different species. In a particular embodiment, the MSC's from which the EVs are derived are allogeneic.

### F. Antibody

In some aspects, the Fli-1 inhibitor is a Fli-1 antibody or fragment thereof that binds to at least a portion of Fli-1 protein and inhibits Fli-1 signaling. The antibody may be selected from the group consisting of a chimeric antibody, an affinity matured antibody, a polyclonal antibody, a monoclonal antibody, a humanized antibody, a human antibody, or an antigen-binding antibody fragment or a natural or synthetic ligand. Preferably, the Fli-1 antibody is a monoclonal antibody or a humanized antibody. In some aspects, the Fli-1 antibody is a monoclonal antibody as described in Zhang et al. (Zhang et al., Hybridoma, 1995) or a Fli-1 polyclonal antibody (e.g., Proteintech Fli-1 Antibody 11347-1-AP).

As used herein, the term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. The antibody may be a bi-specific antibody. In exemplary embodiments, antibodies used with the methods and compositions described herein are derivatives of the IgG class. The term antibody also refers to antigen-binding antibody fragments. Examples of such antibody fragments include, but are not limited to, Fab, Fabÿ, F(abÿ)2, scFv, Fv, dsFv diabody, and Fd fragments. Antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 10 amino acids and more typically will comprise at least about 200 amino acids.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

Examples of antibody fragments suitable for the present embodiments include, without limitation: (i) the Fab fragment, consisting of VL, VH, CL, and CH1 domains; (ii) the “Fd” fragment consisting of the VH and CH1 domains; (iii) the “Fv” fragment consisting of the VL and VH domains of a single antibody; (iv) the “dAb” fragment, which consists of a VH domain; (v) isolated CDR regions; (vi) F(ab′)2 fragments, a bivalent fragment comprising two linked Fab fragments; (vii) single chain Fv molecules (“scFv”), wherein a VH domain and a VL domain are linked by a peptide linker that allows the two domains to associate to form a binding domain; (viii) bi-specific single chain Fv dimers (see U.S. Pat. No. 5,091,513); and (ix) diabodies, multivalent or multispecific fragments constructed by gene fusion (US Patent App. Pub. 20050214860). Fv, scFv, or diabody molecules may be stabilized by the incorporation of disulphide bridges linking the VH and VL domains. Minibodies comprising a scFv joined to a CH3 domain may also be made.

Antibody-like binding peptidomimetics are also contemplated in embodiments. Liu et al. (2003) describe “antibody like binding peptidomimetics” (ABiPs), which are peptides that act as pared-down antibodies and have certain advantages of longer serum half-life as well as less cumbersome synthesis methods.

Animals may be inoculated with an antigen, such as a Fli-1 extracellular domain (ECD) protein, in order to produce antibodies specific for Fli-1. Frequently an antigen is bound or conjugated to another molecule to enhance the immune response. As used herein, a conjugate is any peptide, polypeptide, protein, or non-proteinaceous substance bound to an antigen that is used to elicit an immune response in an animal. Antibodies produced in an animal in response to antigen inoculation comprise a variety of non-identical molecules (polyclonal antibodies) made from a variety of individual antibody producing B lymphocytes. A polyclonal antibody is a mixed population of antibody species, each of which may recognize a different epitope on the same antigen. Given the correct conditions for polyclonal antibody production in an animal, most of the antibodies in the animal's serum will recognize the collective epitopes on the antigenic compound to which the animal has been immunized. This specificity is further enhanced by affinity purification to select only those antibodies that recognize the antigen or epitope of interest.

A monoclonal antibody is a single species of antibody wherein every antibody molecule recognizes the same epitope because all antibody producing cells are derived from a single B-lymphocyte cell line. The methods for generating monoclonal antibodies (MAbs) generally begin along the same lines as those for preparing polyclonal antibodies. In some embodiments, rodents such as mice and rats are used in generating monoclonal antibodies. In some embodiments, rabbit, sheep, or frog cells are used in generating monoclonal antibodies. The use of rats is well known and may provide certain advantages. Mice (e.g., BALB/c mice) are routinely used and generally give a high percentage of stable fusions.

Hybridoma technology involves the fusion of a single B lymphocyte from a mouse previously immunized with a Fli-1 antigen with an immortal myeloma cell (usually mouse myeloma). This technology provides a method to propagate a single antibody-producing cell for an indefinite number of generations, such that unlimited quantities of structurally identical antibodies having the same antigen or epitope specificity (monoclonal antibodies) may be produced.

Plasma B cells (CD45+CD5−CD19+) may be isolated from freshly prepared rabbit peripheral blood mononuclear cells of immunized rabbits and further selected for Fli-1 binding cells. After enrichment of antibody producing B cells, total RNA may be isolated and cDNA synthesized. DNA sequences of antibody variable regions from both heavy chains and light chains may be amplified, constructed into a phage display Fab expression vector, and transformed into E. coli. Fli-1 specific binding Fab may be selected out through multiple rounds enrichment panning and sequenced. Selected Fli-1 binding hits may be expressed as full-length IgG in rabbit and rabbit/human chimeric forms using a mammalian expression vector system in human embryonic kidney (HEK293) cells (Invitrogen) and purified using a protein G resin with a fast protein liquid chromatography (FPLC) separation unit.

In one embodiment, the antibody is a chimeric antibody, for example, an antibody comprising antigen binding sequences from a non-human donor grafted to a heterologous non-human, human, or humanized sequence (e.g., framework and/or constant domain sequences). Methods have been developed to replace light and heavy chain constant domains of the monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see U.S. Pat. Nos. 5,091,513 and 6,881,557). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

Methods for producing polyclonal antibodies in various animal species, as well as for producing monoclonal antibodies of various types, including humanized, chimeric, and fully human, are well known in the art and highly predictable. For example, the following U.S. patents and patent applications provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024. All patents, patent application publications, and other publications cited herein and therein are hereby incorporated by reference in the present application.

Antibodies may be produced from any animal source, including birds and mammals. Preferably, the antibodies are ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by reference.

It is fully expected that antibodies to Fli-1 will have the ability to neutralize or counteract the effects of Fli-1 regardless of the animal species, monoclonal cell line, or other source of the antibody. Certain animal species may be less preferable for generating therapeutic antibodies because they may be more likely to cause allergic response due to activation of the complement system through the “Fc” portion of the antibody. However, whole antibodies may be enzymatically digested into “Fc” (complement binding) fragment, and into antibody fragments having the binding domain or CDR. Removal of the Fc portion reduces the likelihood that the antigen antibody fragment will elicit an undesirable immunological response, and thus, antibodies without Fc may be preferential for prophylactic or therapeutic treatments. As described above, antibodies may also be constructed so as to be chimeric or partially or fully human, so as to reduce or eliminate the adverse immunological consequences resulting from administering to an animal an antibody that has been produced in, or has sequences from, other species.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria. It is also contemplated that a bacteria containing such a variant may be implemented in compositions and methods. Consequently, a protein need not be isolated.

It is contemplated that in compositions there is between about 0.001 mg and about 10 mg of total polypeptide, peptide, and/or protein per ml. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% may be an antibody that binds Fli-1.

An antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

Embodiments provide antibodies and antibody-like molecules against Fli-1, polypeptides and peptides that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules that have been attached to antibodies include toxins, therapeutic enzymes, antibiotics, radio-labeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody. Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

The present antibody may be conjugated to an imaging or diagnostic agent.

A “therapeutic agent” as used herein refers to any agent that can be administered to a subject for the purpose of obtaining a therapeutic benefit of a disease or health-related condition. For example, antibodies conjugated to a therapeutic agent may be administered to a subject for the purpose of reducing the size of a tumor, reducing or inhibiting local invasiveness of a tumor, or reducing the risk of development of metastases.

A “diagnostic agent” or “imaging agent” (referred to interchangeably) as used herein refers to any agent that can be administered to a subject for the purpose of diagnosing a disease or health-related condition in a subject. Diagnosis may involve determining whether a disease is present, whether a disease has progressed, or any change in disease state.

The therapeutic or diagnostic agent may be a small molecule, a peptide, a protein, a polypeptide, an antibody, an antibody fragment, a DNA, or an RNA.

### G. Delivery

The Fli-1 antibody may be delivered as nanoparticles, liposomes, or polymeric nanocarriers. In some aspects, the antibody may be loaded in polymeric micelles or nanogels.

For example, the antibody may be delivered by employing polymer nanogels. The antibody may be encapsulated inside a nanogel by conjugation using a redox-sensitive traceless linker (Sui et al., Adv. Funct. Mater., 2021; incorporated herein by reference in its entirety).

The “Trim-Away” method, which utilizes antibodies to degrade endogenous proteins in mammalian cells without prior modification of the genome or mRNA (Clift et al., Nature Protocols, 2018; incorporated by reference in its entirety) may be used. The mechanism of Trim-Away involves the intracellular antibody receptor TRIM21, which is an E3 ubiquitin ligase that binds to the Fc domain of antibodies, and TRIM21 is commonly expressed in various cell types because of its indispensable physiological role. During Trim-Away, the target protein is bound by the antibody, followed by TRIM21-mediated ubiquitination to generate a protein complex, which is subsequently degraded in the proteasome.

In some aspects, Poly[(2-(pyridin-2-yldisulfanyl)ethyl acrylate)-co-[poly(ethylene glycol)]] (PDA-PEG) polymer bearing p-nitrophenylcarbonate (NPC) moieties in side chains may be synthesized by attaching NPC to beta-mercaptoethanol modified PDA-PEG (PDA-PEG-BME). PDA-PEG-BME polymer may be prepared through a thiol-disulfide exchange reaction between polymer PDA-PEG and 2-mercaptoethanol (BME). Briefly, PDA-PEG polymer, synthesized with the PDA:PEG at the ratio of 1:1 and characterized by 1H NMR and gel permeation chromatography (Mw: 21.5 kDa, PDI: 1.3), may be first dissolved in DCM supplemented with glacial acetic acid. After that, BME dissolved in DCM may be added dropwise under stirring at room temperature. The reaction may be stopped after being processed in the dark overnight. The reaction mixture may be purified by precipitating in ice-cold diethyl ether for three times. The protein/antibody may be reacted with PDA-PEG-NPC to produce protein/antibody conjugated polymers, in which the NPCs are replaced by the reactive lysine groups of the protein/antibody. The redox-sensitive linker between the antibody and the polymer backbone is self-immolative and readily cleaved by reducing agents such as GSH. The resulted polymers may be fabricated into polymer nanogels via the crosslinking reaction induced by tris (2-carboxyethyl) phosphine (TCEP). For example, to a pre-cooled solution of 33 mg polymer PDA-PEG-NPC in 1 mL DMSO, 11 mg protein/antibody dissolved in 1 mL PBS buffer (pH 8.5) may be added dropwise at 4° C. under vigorous stirring, and the resulted solution may be stirred for 48 h at 4° C. in the dark. The process of reaction may be monitored by measuring the absorbance of released side product 4-nitrophenol at 400 nm using UV-Vis spectroscopy. When the reaction is completed, 2.6 mg TCEP and 1.1 mg ethylenediamine dissolved in 0.2 mL pre-cooled deionized water may be added for crosslinking and the solution was stirred for 24 h at 4° C. Then the produced nanogels may be purified through dialysis in Spectra/Por® dialysis tube (regenerated cellulose, MWCO: 100 kDa) against PBS buffer for 48 h at 4° C. The final nanogels may be stored in PBS (pH 7.4) at 4° C. for use. For the nanogels modified with Cy3, polymer PDA-PEG-Cy3 was mixed into the reaction solution before the crosslinking step.

The antibody-loaded nanogel can enter cell through receptor mediated endocytosis and subsequently release its payload due to the elevated intracellular glutathione (GSH). After that, the antibody binds to its target protein and TRIM21 to yield a protein/antibody/TRIM21 complex, which can be degraded by a proteasome-mediated cellular protein degradation machinery.

### III. FORMULATION AND ADMINISTRATION

The present disclosure provides pharmaceutical compositions comprising inhibitors of Fli-1, such as antisense oligonucleotides or antibody. Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals. One will generally desire to employ appropriate salts, buffers, and lipids to render delivery of the oligonucleotides to allow for uptake by target cells. Such methods and compositions are well known in the art, for example, as disclosed in U.S. Pat. Nos. 6,747,014 and 6,753,423, incorporated by reference herein. Compositions of the present disclosure comprise an effective amount of the oligonucleotide to cells, dissolved or dispersed in a pharmaceutically acceptable carrier or medium.

The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents, liposomes, cationic lipid formulations, microbubble nanoparticles, and the like. Except insofar as any conventional media or agent is incompatible with the vectors or cells of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. Ocular administration includes topical eye drops, intracameral injection, or intravitreal injection to target the cornea including corneal endothelial cell layer. Alternatively, administration may be oral, nasal, buccal, or topical. Alternatively, administration may be by intradermal, subcutaneous, intramuscular, intraperitoneal or intravenous injection, or introduction into the CNS, such as into spinal fluid. Such compositions would normally be administered as pharmaceutically acceptable compositions, described supra.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, lipids, nanoparticles, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the oligonucleotides of the present disclosure may be incorporated with excipients. The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Of particular interest to the present disclosure is the use of lipid delivery vehicles. Lipid vehicles encompass micelles, microemulsions, macroemulsions, liposomes, and similar carriers. The term micelle refers to colloidal aggregates of amphipathic (surfactant) molecules that are formed at a well-defined concentration known as the critical micelle concentration. Micelles are oriented with the nonpolar portions at the interior and the polar portions at the exterior surface, exposed to water. The typical number of aggregated molecules in a micelle (aggregation number) is 50 to 100. Microemulsions are essentially swollen micelles, although not all micellar solutions can be swollen to form microemulsions. Microemulsions are thermodynamically stable, are formed spontaneously, and contain particles that are extremely small. Droplet diameters in microemulsions typically range from 10 100 nm. In contrast, the term macroemulsions refers to droplets with diameters greater than 100 nm. Liposomes are closed lipid vesicles comprising lipid bilayers that encircle aqueous interiors. Liposomes typically have diameters of 25 nm to 1 μm.

In one embodiment of a liposome formulation, the principal lipid of the vehicle may be phosphatidylcholine. Other useful lipids include various natural (e.g., tissue derived L-α-phosphatidyl: egg yolk, heart, brain, liver, soybean) and/or synthetic (e.g., saturated and unsaturated 1,2-diacyl-SN-glycero-3-phosphocholines, 1-acyl-2-acyl-SN-glycero-3-phosphocholines, 1,2-diheptanoyl-SN-glycero-3-phosphocholine) derivatives of the same. Such lipids can be used alone, or in combination with a secondary lipid. Such secondary helper lipids may be non-ionic or uncharged at physiological pH, including non-ionic lipids such as cholesterol and DOPE (1,2-dioleolylglyceryl phosphatidylethanolamine). The molar ratio of a phospholipid to helper lipid can range from about 3:1 to about 1:1, from about 1.5:1 to about 1:1, and about 1:1.

Exemplary amounts of lipid constituents used for the production of the liposome include, for instance, 0.3 to 1 mol or 0.4 to 0.6 mol of cholesterol; 0.01 to 0.2 mol or 0.02 to 0.1 mol of phosphatidylethanolamine; 0.0 to 0.4 mol or 0-0.15 mol of phosphatidic acid per 1 mol of phosphatidylcholine.

Liposomes can be constructed by well-known techniques. Lipids are typically dissolved in chloroform and spread in a thin film over the surface of a tube or flask by rotary evaporation. If liposomes comprised of a mixture of lipids are desired, the individual components are mixed in the original chloroform solution. After the organic solvent has been eliminated, a phase consisting of water optionally containing buffer and/or electrolyte is added and the vessel agitated to suspend the lipid. Optionally, the suspension is then subjected to ultrasound, either in an ultrasonic bath or with a probe sonicator, until the particles are reduced in size and the suspension is of the desired clarity. For transfection, the aqueous phase is typically distilled water and the suspension is sonicated until nearly clear, which requires several minutes depending upon conditions, kind, and quality of the sonicator. Commonly, lipid concentrations are 1 mg/ml of aqueous phase, but could be higher or lower by about a factor of ten.

Lipids, from which the solvents have been removed, can be emulsified by the use of a homogenizer, lyophilized, and melted to obtain multilamellar liposomes. Alternatively, unilamellar liposomes can be produced by the reverse phase evaporation method (Szoka and Papahadjopoulos, 1978). Unilamellar vesicles can also be prepared by sonication or extrusion. Sonication is generally performed with a bath-type sonifier, such as a Branson tip sonifier (G. Heinemann Ultrashall and Labortechnik, Schwabisch Gmund, Germany) at a controlled temperature as determined by the melting point of the lipid. Extrusion may be carried out by biomembrane extruders, such as the Lipex Biomembrane Extruder (Northern Lipids Inc, Vancouver, British Columbia, Canada). Defined pore size in the extrusion filters may generate unilamellar liposomal vesicles of specific sizes. The liposomes can also be formed by extrusion through an asymmetric ceramic filter, such as a Ceraflow Microfilter (commercially available from the Norton Company, Worcester, Mass.).

Liposomes can be extruded through a small-pore polycarbonate membrane or an asymmetric ceramic membrane to yield a well-defined size distribution. Typically, a suspension is cycled through the membrane one or more times until the desired liposome size distribution is achieved. The liposomes may be extruded through successively smaller-pore membranes, to achieve a gradual reduction in liposome size. For use in the present disclosure, liposomes have a size of about 0.05 microns to about 0.5 microns, or having a size of about 0.05 to about 0.2 microns.

The term conjugate as used herein refers to an oligonucleotide which is covalently linked to a non-nucleotide moiety (conjugate moiety or region C or third region). Conjugation of the present oligonucleotides to one or more non-nucleotide moieties may improve the pharmacology of the oligonucleotide, e.g. by affecting the activity, cellular distribution, cellular uptake or stability of the oligonucleotide. In some embodiments the conjugate moiety modify or enhance the pharmacokinetic properties of the oligonucleotide by improving cellular distribution, bioavailability, metabolism, excretion, permeability, and/or cellular uptake of the oligonucleotide. In particular, the conjugate may target the oligonucleotide to a specific organ, tissue or cell type and thereby enhance the effectiveness of the oligonucleotide in that organ, tissue or cell type. At the same time the conjugate may serve to reduce activity of the oligonucleotide in non-target cell types, tissues or organs, e.g. off target activity or activity in non-target cell types, tissues or organs.

WO 93/07883 and WO 2013/033230 provides suitable conjugate moieties, which are hereby incorporated by reference. Further suitable conjugate moieties are those capable of binding to the asialoglycoprotein receptor (ASGPR). In particular, tri-valent N-acetylgalactosamine conjugate moieties are suitable for binding to the ASGPR, see for example WO 2014/076196, WO 2014/207232 and WO 2014/179620 (hereby incorporated by reference). Such conjugates serve to enhance uptake of the oligonucleotide to the liver while reducing its presence in the kidney, thereby increasing the liver/kidney ratio of a conjugated oligonucleotide compared to the unconjugated version of the same oligonucleotide.

Oligonucleotide conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103, each of which is incorporated herein by reference in its entirety.

In an embodiment, the non-nucleotide moiety (conjugate moiety) is selected from the group consisting of carbohydrates, cell surface receptor ligands, drug substances, hormones, lipophilic substances, polymers, proteins, peptides, toxins (e.g., bacterial toxins), vitamins, viral proteins (e.g., capsids) or combinations thereof.

### IV. METHODS OF TREATMENT

In particular, compositions that may be used in preventing and/or treating inflammatory disease in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., retarding pericyte loss, ameliorating cognitive deficits, reducing Aβ deposition, and decelerating BBB breakdown). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms of the infection or cancer and other drugs being administered concurrently.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

In certain embodiments, oligonucleotide compounds and compositions are delivered to the brain of a subject. In certain embodiments, an oligonucleotide composition is delivered once every week, every two weeks, every month, every two months, every 90 days, every 3 months, every 6 months, twice a year or once a year.

In one embodiment, the subject has an inflammatory condition or autoimmune disease. Non-limiting examples of inflammatory diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or membranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis.

The term “neurodegenerative disease or disorder” and “neurological disorders” encompass a disease or disorder in which the peripheral nervous system or the central nervous system is principally involved. The compounds, compositions, and methods provided herein may be used in the treatment of neurological or neurodegenerative diseases and disorders. As used herein, the terms “neurodegenerative disease”, “neurodegenerative disorder” “neurological disease”, and “neurological disorder” are used interchangeably.

Examples of neurological disorders or diseases include, but are not limited to chronic neurological diseases such as diabetic peripheral neuropathy (including third nerve palsy, mononeuropathy, mononeuropathy multiplex, diabetic amyotrophy, autonomic neuropathy and thoracoabdominal neuropathy), Alzheimer's disease, age-related memory loss, senility, age-related dementia, Pick's disease, diffuse Lewy body disease, progressive supranuclear palsy (Steel-Richardson syndrome), multisystem degeneration (Shy-Drager syndrome), motor neuron diseases including amyotrophic lateral sclerosis (“ALS”), degenerative ataxias, cortical 0 basal degeneration, ALS-Parkinson's-Dementia complex of Guam, subacute sclerosing panencephalitis, Huntington's disease, Parkinson's disease, multiple sclerosis (“MS”), synucleinopathies, primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Wernicke-15 Korsakoff's related dementia (alcohol induced dementia), Werdnig-Hoffmann disease, Kugelberg-Welander disease, Tay-Sach's disease, Sandhoff disease, familial spastic disease, Wohifart-Kugelberg-Welander disease, spastic paraparesis, progressive multifocal leukoencephalopathy, and prion diseases (including Creutzfeldt-Jakob, Gerstmann-Straussler-Scheinker disease, Kuru and fatal familial insomnia). Other conditions also included within the methods of the present disclosure include age-related dementia and other dementias, and conditions with memory loss including vascular dementia, diffuse white matter disease (Binswanger's disease), dementia of endocrine or metabolic origin, dementia of head trauma and diffuse brain damage, dementia pugilistica, and frontal lobe dementia. Also other neurodegenerative disorders resulting from cerebral ischemia or infarction including embolic occlusion and thrombotic occlusion as well as intracranial hemorrhage of any type (including, but not limited to, epidural, subdural, subarachnoid, and intracerebral), and intracranial and intravertebral lesions (including, but not limited to, contusion, penetration, shear, compression, and laceration). Thus, the term also encompasses acute neurodegenerative disorders such as those involving stroke, traumatic brain injury, schizophrenia, peripheral nerve damage, hypoglycemia, spinal cord injury, epilepsy, and anoxia and hypoxia.

### V. COMBINATION THERAPIES

It is common in many fields of medicine to treat a disease with multiple therapeutic modalities, often called “combination therapies.” In the present application, combination therapies may be used to treat inflammatory or neurodegenerative disorders. Such combinations will include the oligonucleotides according to the present disclosure, along with one or more “standard” therapeutic modalities.

Thus, to treat inflammatory or neurodegenerative disorders, one may contact a target cell in subject with an oligonucleotide and at least one other therapy. These therapies would be provided in a combined amount effective to achieve a reduction in one or more disease parameter. This process may involve contacting the subjects with the both agents/therapies at the same time, e.g., using a single composition or pharmacological formulation that includes both agents, or by contacting the cell/subject with two distinct compositions or formulations, at the same time, wherein one composition includes the oligonucleotide and the other includes the other agent.

Alternatively, the oligonucleotide may precede or follow the other treatment by intervals ranging from minutes to weeks. One would generally ensure that a significant period of time did not expire between each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the oligonucleotide or the other therapy will be desired. Various combinations may be employed, where the Fli-1 inhibitor is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. Therapies to suppress brain inflammation or increase removal of amyloid β-peptide can be used in combination with the oligonucleotide therapies described herein.

### VI. KITS

In various aspects of the embodiments, a kit is envisioned containing therapeutic agents and/or other therapeutic and delivery agents. In some embodiments, the present embodiments contemplate a kit for preparing and/or administering a composition (e.g., a Fli-1 inhibitor, such as a gapmer or antibody) of the embodiments. The kit may comprise one or more sealed vials containing any of the pharmaceutical compositions of the present embodiments. The kit may include, for example, engineered antibodies as well as reagents to prepare, formulate, and/or administer the components of the embodiments or perform one or more steps of the inventive methods. In some embodiments, the kit may also comprise a suitable container, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### VII. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Suppression of Fli-1 Protects Against Pericyte Loss and Cognitive Deficits in Alzheimer's Disease

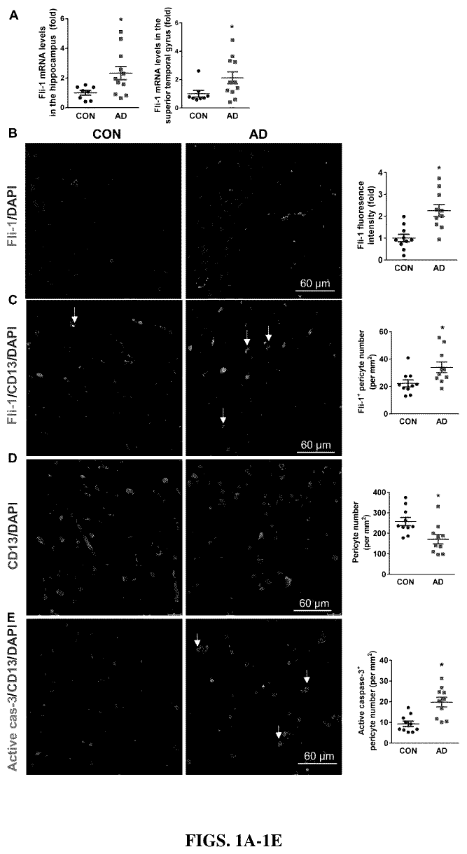

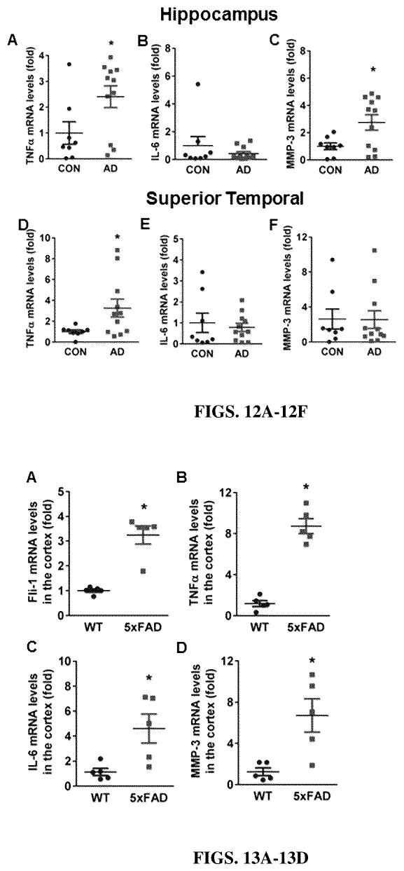

Increased Fli-1 corresponded with pericyte loss in the hippocampus from postmortem brains of AD patients. To investigate the possible role of Fli-1 in AD, Fli-1 expression levels were first detected in the patients with AD. The demographics for the human subjects in the study are shown in Table 1. Fli-1 mRNA levels determined by real-time PCR were significantly increased in the hippocampus and superior temporal gyrus of AD patients (N=8-11, FIG. 1A; p<0.05); these data are from a subcohort of human subjects with AD diagnosis (age mean±SD: 83.7±6.7 years; 7 females/4 males; PMI mean±SD: 5.6±1.7 h) and compared to cognitively normal controls (age mean±SD: 73.2±19.8 years; 4 females/4 males; PMI mean±SD: 9.2±3.3 h). Similarly, the Fli-1 levels determined by immunostaining in the hippocampus were also significantly increased in AD patients (N=10) compared with cognitively normal controls (N=10, FIG. 1B; p<0.05). The immunostaining was performed on postmortem brains isolated from another subcohort of human subjects with AD diagnosis (age mean±SD: 77.2±2.7 years; 4 females/6 males; PMI mean±SD: 14.4±6.3 h) and compared to cognitively normal controls (age mean±SD: 76.2±11.5 years; 5 females/5 males; PMI mean±SD: 15.2±9.1 h). Expression of TNFα and MMP3 but not IL-6 was increased in the hippocampus; however, increased TNFα expression was only observed in the superior temporal gyrus of AD patients (FIG. 12; same cohort as in FIG. 1A). In addition, Fli-1 levels were significantly (p<0.05) upregulated within pericytes in the hippocampus of AD patients (N=10, FIG. 1C; same cohort as in FIG. 1B) as evidenced by increased Fli-1⁺ pericyte number. However, the absolute number of pericytes (CD13-positive) in the hippocampus was significantly (p<0.05) decreased by 34% in AD patients compared with cognitively normal controls (N=10, FIG. 1D; same cohort as in FIG. 1B). Quantification of active caspase-3⁺ pericyte number confirmed pericyte apoptosis in the hippocampus, which was significantly (p<0.05) higher in AD patients (N=10, FIG. 1E; same cohort as in FIG. 1B).

TABLE 1
Demographic data for all human brain donors

| N = 38. | Cognitively normal controls | | Alzheimer's disease | |
|---|---|---|---|---|
| Subject size | 17 | | 21 | |
| Females (%) | 9F | (52.9%) | 11F | (52.4%) |
| Age ± SD (years) | 73.9 ± 15.2 | | 80.6 ± 6.1 | |
| Race (%) | 15W | (88.2%) | 19W | (90.5%) |
| | 1B | (5.9%) | 2 N/A | (9.5%) |
| | 1A | (5.9%) | | |

| | | |
|---|---|---|
| PMI ± SD (h) | 12.2 ± 7.7 | 9.8 ± 6.3 |

F female, M male, SD standard deviation, W White, B Black, A Asian, N/A not available, PMI post-mortem interval, Values are presented as mean ± SD.

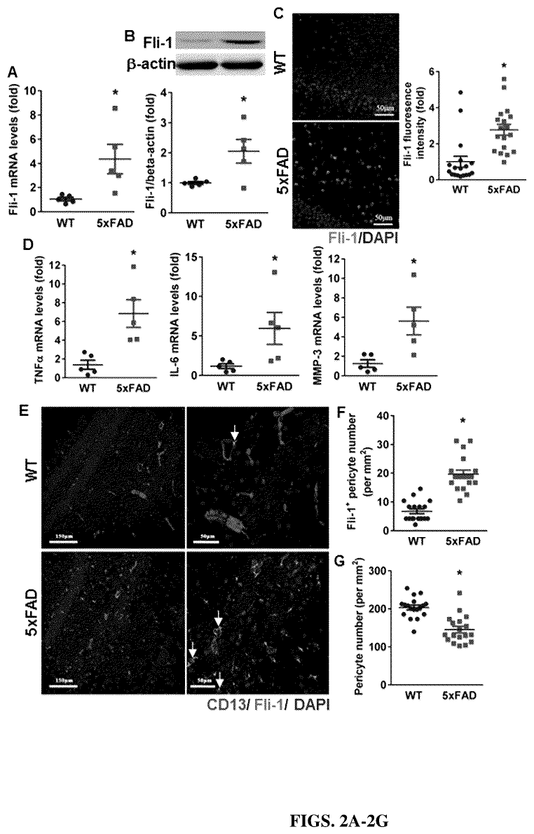

Increased Fli-1 and pericyte loss in the hippocampus of 5×FAD mice. Fli-1 levels and pericyte loss in the hippocampus was further investigated in a transgenic AD-mice model (5×FAD) compared to their age- and sex-matched WT littermates (6.5-months-old). A significant increase of Fli-1 mRNA and protein levels was observed in the hippocampus of 5×FAD mice compared with the WT mice (FIGS. 2A, 2B and 2C; p<0.05). Fli-1 mRNA levels were also significantly increased in the cortex of AD mice (FIG. 13A; p<0.05). Increased expression of inflammatory mediators and MMPs including TNFα, IL-6 and MMP3, which are implicated in AD development 23.24, was observed in the hippocampus and cortex of 5×FAD mice (FIGS. 2D, 13B, 13C, and 13D; p<0.05). Increased Fli-1 within pericytes, and decreased pericyte number by 29% in the hippocampus of 5×FAD mice was confirmed by immunostaining (FIGS. 2E, 2F and 2G; p<0.05) analogous to observations in human brain tissues.

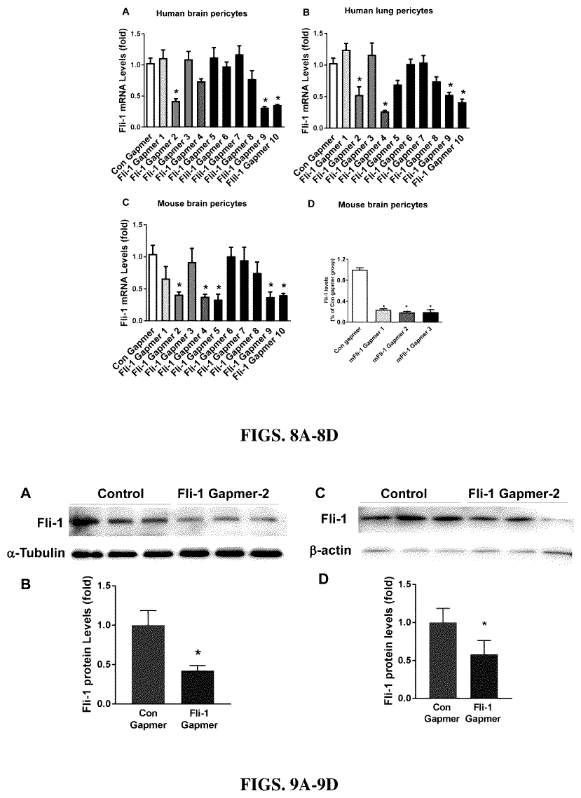

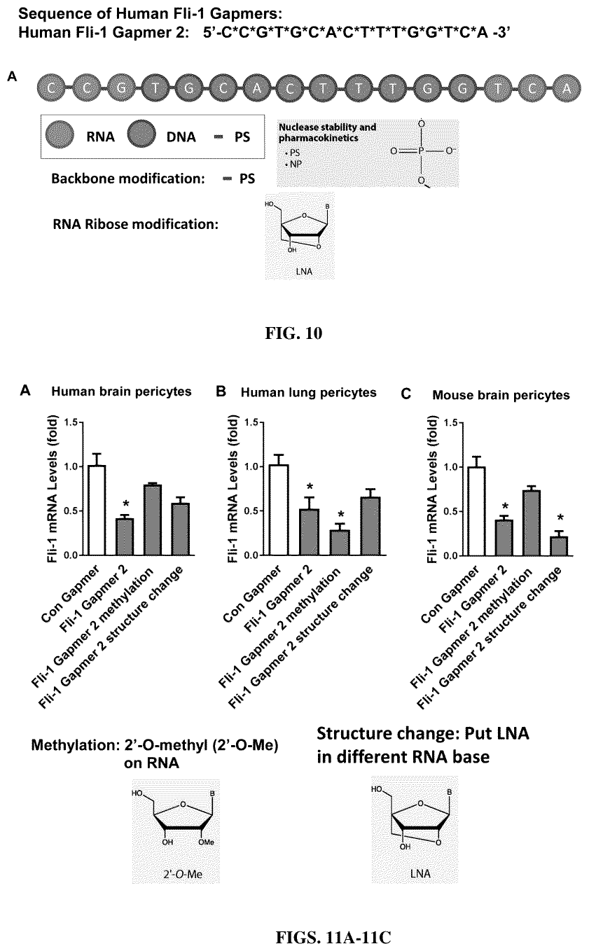

To investigate the role of Fli-1 in pericyte dysfunction and AD development, five antisense oligonucleotide Gapmers targeting human Fli-1 and three Gapmers targeting mouse Fli-1 were designed. Antisense Gapmers are based on locked nucleic acid (LNA) technology, which are stable in circulation, can be taken up by cells without the need for transfection reagents, and last at least 14 days in vivo. It was demonstrated that human Fli-1 Gapmers 2, 9, and 10 effectively suppressed Fli-1 levels in human brain and lung pericytes and mouse brain pericytes (FIGS. 8A-8C), and Fli-1 Gapmer 2 also suppresses Fli-1 protein levels in human and mouse pericytes (FIG. 9). Mouse Fli-1 Gapmers 1, 2, and 3 suppressed Fli-1 levels in mouse brain pericytes. (FIG. 8D).

Details of human Fli-1 Gapmer design. The sequence of human Fli-1 Gapmer 2 (SEQ ID NO: 2) is shown in FIG. 11. The details of human Fli-1 Gapmer 2 are also shown. In DNA and RNA, nucleotides are linked through a phosphodiester backbone, which is rapidly cleaved by endo- and exonucleases in serum and in cells. The engineered Gapmer has a phosphorothioate (PS) backbone modification, which confers its resistance to degradation by nucleases. PS also improves the pharmacokinetic properties that allow them to bind to serum albumin and enter into cells. The DNA sequence allows the Gapmer to bind to the target and maintain RNase H-mediated cleavage of RNA. The RNA sequence was subjected to locked nucleic acid (LNA) modifications. LNA oligonucleotides exhibit unprecedented thermal stability when hybridized to a complementary DNA or RNA strand.

Modification of Fli-1 Gapmers. The Fli-1 Gapmer 2 was modified with 2′-O-methyl (2′-O-Me) modification and the LNA was put on different RNA base. It was found that the 2′-O-Me modification was more effective in human lung pericytes and different LNA position worked better in mouse brain pericyte (FIG. 11). Other modifications of Fli-1 Gapmer 2 such as 2′-O-methoxyethyl (2′-O-MOE) and 2′-fluoro (2′-F) modifications may be used.

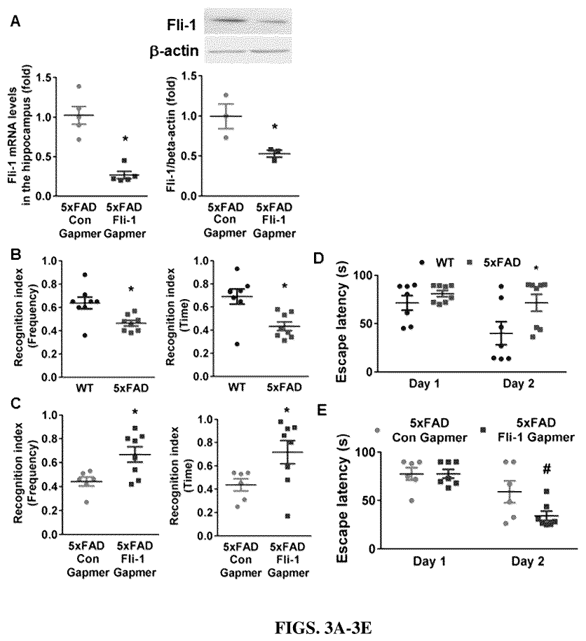

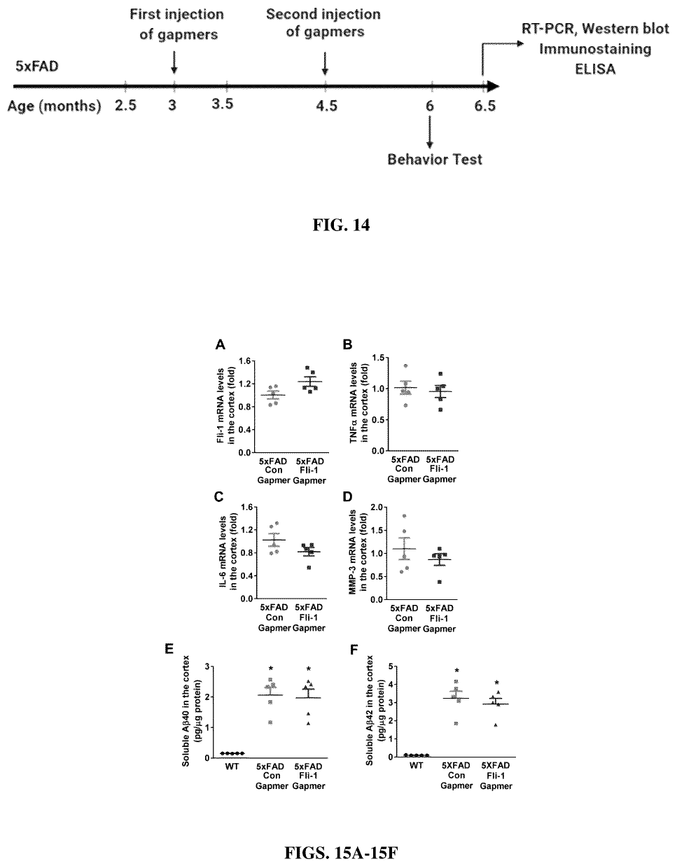

Fli-1 Gapmer treatment ameliorates cognitive deficits in 5×FAD mice. To further investigate the possible association between Fli-1 and cognitive deficits in AD, Fli-1 was knocked down by intrahippocampal injection of Fli-1 antisense oligonucleotide Gapmers in 5×FAD mice at ages 3 and 4.5-months-old (FIG. 14). Decreased Fli-1 mRNA and protein levels in the hippocampus but not in the cortex of 6.5-month-old 5×FAD mice were confirmed in the Fli-1 Gapmer group (FIGS. 3A and 15). Significant impairment in working memory and spatial memory has been observed in 5×FAD mice (Maiti et al., 2021) (Jawhar et al., 2012). To investigate the impairment in working memory, novel object recognition (NOR) test was performed in 6-month-old 5×FAD mice. It was demonstrated that 6-month-old 5×FAD mice had significantly less interactions with the novel object (frequency recognition index) and spent significantly less time exploring the novel object (time recognition index) than WT mice (FIG. 3B; p<0.05), indicating the impairment in working memory of 5×FAD mice. However, Fli-1 Gapmer administration significantly improved the recognition index of frequency and time in 5×FAD mice compared with control Gapmer group (FIG. 3C; p<0.05). Morris water maze (MWM) test was used to explore spatial memory limitations in 6-month-old 5×FAD mice. After training trials on Day 1, the escape latency time on Day 2 of the 5×FAD mice was significantly longer than that of the WT mice (FIG. 3D; p<0.05), indicating the impairment in spatial memory of 5×FAD mice. However, the escape latency of the 5×FAD mice on Day 2 of the Fli-1 Gapmer group was significantly reduced compared with that of the control Gapmer group (FIG. 3E; p<0.05).

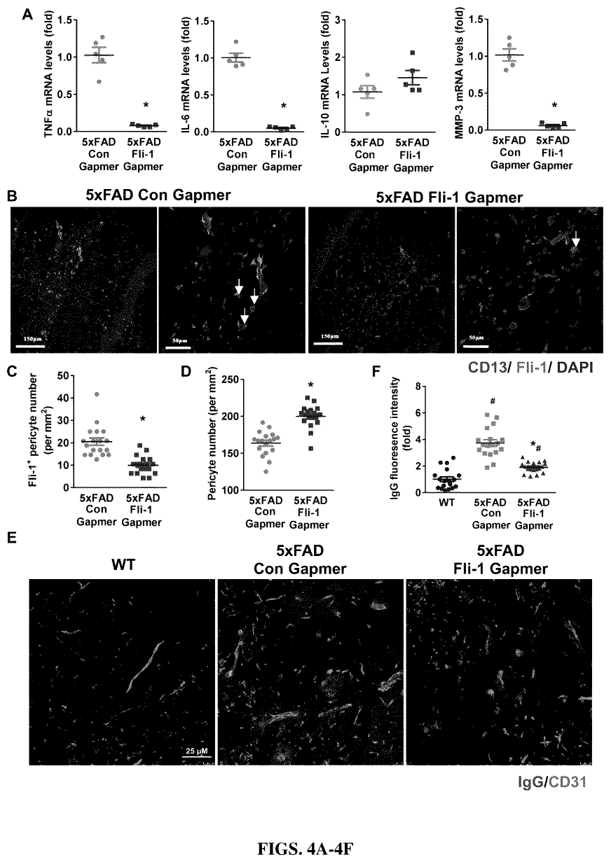

Fli-1 Gapmer treatment reduces pericyte loss and improves BBB dysfunction in the hippocampus of 5×FAD mice. To further investigate the possible role of Fli-1 in AD pathology, inflammatory mediators, MMP, pericyte loss and BBB dysfunction were assessed in 5×FAD mice treated with Fli-1 Gapmer or control Gapmer at ages 6.5-months-old. As revealed in FIG. 4A, TNFα, IL-6 and MMP3 expression in the hippocampus of 5×FAD mice were significantly (p<0.05) decreased in the Fli-1 Gapmer group. As intrahippocampal injection of Fli-1 Gapmer did not affect the Fli-1 levels in the cortex, unchanged TNFα, IL-6 and MMP3 expression was consistently observed in the cortex of the Fli-1 Gapmer group (FIGS. 15B, 15C, and 15D). Fli-1 expression decreased within pericytes although the overall pericyte number increased by 22% in the hippocampus of 5×FAD mice treated with Fli-1 Gapmer as compared to the control Gapmer group (FIGS. 4B, 4C and 4D; p<0.05). In addition, inhibition of Fli-1 significantly attenuated the vascular leakage of IgG in the hippocampus of 5×FAD mice suggesting that Fli-1 Gapmer treatment improved BBB dysfunction (FIGS. 4E and 4F; p<0.05).

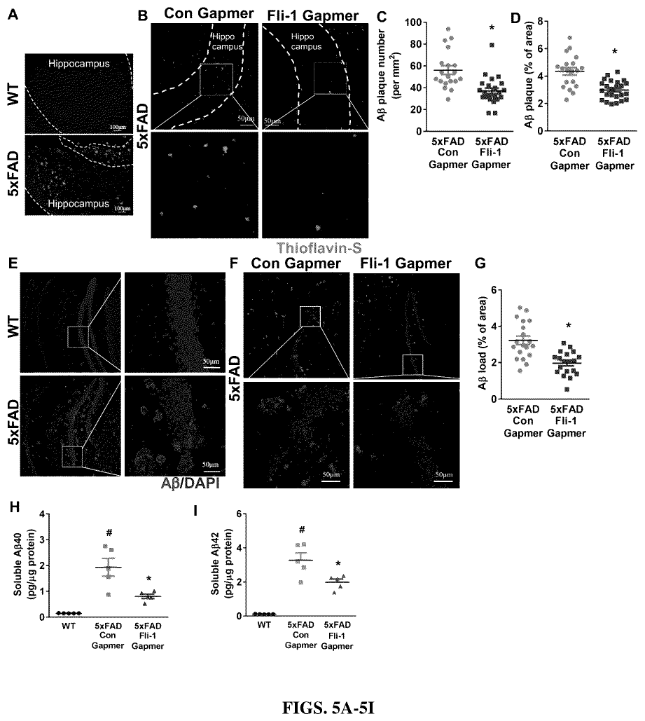

Fli-1 Gapmer treatment attenuates Aβ accumulation in the hippocampus of 5×FAD mice. Pericyte loss has been associated with increased BBB breakdown and Aβ deposition in the hippocampus from AD patients and animal models (Wu et al., 2019) (Sengillo et al., 2013). Thus, the effect of Fli-1 Gapmer via intrahippocampal injection on Aβ deposition was further determined in 5×FAD mice. By 6.5 months, significant Aβ plaques were observed by Thioflavin-S staining (FIG. 5A) in the hippocampus of 5×FAD mice but were barely found in their WT littermates. Fli-1 Gapmer treatment significantly reduced both the number and area of amyloid plaque deposition in the hippocampus (FIGS. 5B, 5C and 5D; p<0.05). Moreover, Aβ load by human Aβ antibody staining (FIG. 5E) was observed in the hippocampus of 5×FAD mice but was barely found in their WT littermates. 5×FAD mice treated with Fli-1 Gapmer had less Aβ load in the hippocampus compared to the control Gapmer group (FIGS. 5F and 5G; p<0.05). To strengthen these results, soluble human Aβ40 and Aβ42 levels were further determined in the hippocampus by ELISA assay. Consistently, it was found that soluble Aβ40 and Aβ42 levels in the hippocampus were significantly increased in 5×FAD mice but reduced in those injected with Fli-1 Gapmer (FIG. 5H, p<0.05). However, soluble Aβ40 and Aβ42 levels in the cortex were not affected by intrahippocampal injection of Fli-1 Gapmers due to the undisrupted Fli-1 levels (FIG. 15E, F).

Fli-1 Gapmer treatment prevents Aβ accumulation-induced pericyte apoptosis. To investigate the role of Fli-1 in pericyte apoptosis, human brain pericytes were cultured in vitro. Aβ40 or TNFα treatment significantly increased Fli-1 mRNA levels in human brain pericytes (FIG. 6A; p<0.05). Prolonged accumulation of Aβ40 has been reported to cause cell apoptosis of brain pericytes (Wu et al., 2019) (Sagare et al., 2013). Consistently, cell viability assay showed that 7 days of continuous treatment with freshly aggregated Aβ40 treatment significantly decreased pericyte cell viability, and transfection with Fli-1 Gapmer treatment significantly decreased Fli-1 levels (FIG. 6B; p<0.05) and increased cell viability in brain pericytes (FIG. 6C; p<0.05). Knockdown of Fli-1 significantly reduced Aβ40-induced caspase-3 mRNA levels (FIG. 6D; p<0.05). To assess whether Fli-1 Gapmer-prevented pericyte death is attributed to inhibited apoptosis, TUNEL and active caspase-3 staining, two typical markers of apoptosis induction, were next performed. Indeed, the Aβ40-induced increase of fluorescence intensity of TUNEL and active caspase-3 expression in human brain pericytes was significantly suppressed in Fli-1 Gapmer group compared to the control Gapmer group (FIGS. 6E, 6F and 6G; p<0.05).

AD is the most common neurodegenerative disorder and a leading cause of dementia among elderly people. It accounted for approximately 70% of the 50 million people suffering from dementia worldwide in 2018, and this figure is anticipated to triple by 2050 with associated costs approaching 4 trillion dollars (Iadecola et al., 2019). Pericyte loss in AD and its central role in regulating BBB integrity, Aβ accumulation and cognitive function have been recognized recently and hold promise to therapeutic interventions (Halliday et al., 2016) (Ma et al., 2018) (Tachibana et al., 2018) (Brown et al., 2019) (Wu et al., 2019). In the present study, it was demonstrated for the first time that the antisense oligonucleotide Gapmer-mediated inhibition of Fli-1 alleviates AD progression via its protective effects against pericyte loss. This notion can be supported by our several novel discoveries. First, Fli-1 expression level is increased with pericyte loss in postmortem brains from AD patients and in 5×FAD mice. Second, knockdown of Fli-1 via intrahippocampal injection of antisense Gapmers ameliorates cognitive deficits, pericyte loss, BBB dysfunction and Aβ deposition in 5×FAD mice. Lastly, Fli-1 Gapmer treatment inhibits Aβ accumulation-induced pericyte apoptosis in cultured human brain pericytes.

Inflammatory response is a factor in the development of AD (Cai et al., 2014). As a regulator of inflammation, Fli-1 belongs to the ETS transcription factor family and is expressed in macrophages, B cells, T cells, endothelial cells and pericytes (Li et al., 2018) (Takahashi et al., 2017) (Theisen et al., 2016) (Sato et al., 2014) (Lou et al., 2017). Abnormal Fli-1 expression has been observed in several diseases including ewing sarcoma, systemic lupus erythematosus (SLE), systemic sclerosis and sepsis, and plays a role in the pathological development of these diseases (Li et al., 2018) (Taniguchi et al., 2015) (Kubo et al., 2003) (Zampeli et al., 2017) (Parikh et al., 2020) (Elzi et al., 2015). It has been demonstrated that Fli-1 expression is significantly increased in lung pericytes and contributes to the inflammatory response and vascular leak in a murine model of sepsis (Li et al., 2018). In this study, it was shown that pericyte Fli-1 levels were elevated in the hippocampus of AD patients and in 5×FAD mice along with increased inflammatory mediators TNFα and IL-6 (FIGS. 1, 2 and 12).

Fli-1 Gapmer treatment suppressed BACE1 levels in the hippocampus in 5×FAD mice. Next, Fli-1 or control Gapmers (1 nmol/kg, 1 ul) were injected into both sides of the hippocampus of 5×FAD mice at 3 and 4.5 months of age. WT, 5×FAD, and 5×FAD mice treated with Fli-1 or control Gapmers were sacrificed at 6.5 months of age and beta-site amyloid precursor protein cleaving enzyme 1 (BACE1) levels in the hippocampus were determined by RT-PCR. The data demonstrated that BACE1 mRNA levels were not significantly different between WT and 5×FAD mice, however Fli-1 Gapmer treatment significantly decreased BACE1 levels (FIG. 16). These data suggest that Fli-1 Gapmers effectively suppressed BACE1 levels, which may result in decreased Aβ levels.

Fli-1 Gapmer treatment decreased ICAM1 and VCAM1 levels in the hippocampus in 5×FAD mice. Fli-1 or control Gapmers (1 nmol/kg) were injected into both sides of the hippocampus of 5×FAD mice at 3 and 4.5 months of age. WT, 5×FAD, and 5×FAD mice treated with Fli-1 or control Gapmers were sacrificed at 6.5 months of age. ICAM1 and VCAM1 mRNA levels in the hippocampus were determined by RT-PCR. It was demonstrated that ICAM1 and VCAM1 levels were significantly increased in 5×FAD mice compared to WT mice, suggesting that neutrophil arrest in capillaries, which might be responsible for the reduction in blood flow, occurs in 5×FAD mice (FIG. 17A,B). Interestingly, injection of Fli-1 Gapmers significantly decreased ICAM1 and VCAM1 levels in the hippocampus, suggesting that neutrophil may not arrest in capillaries and cerebral blood flow may be restored (FIG. 17C,D). These data suggest that suppression of Fli-1 restores cerebral blood flow by decreasing adhesion molecule expression.

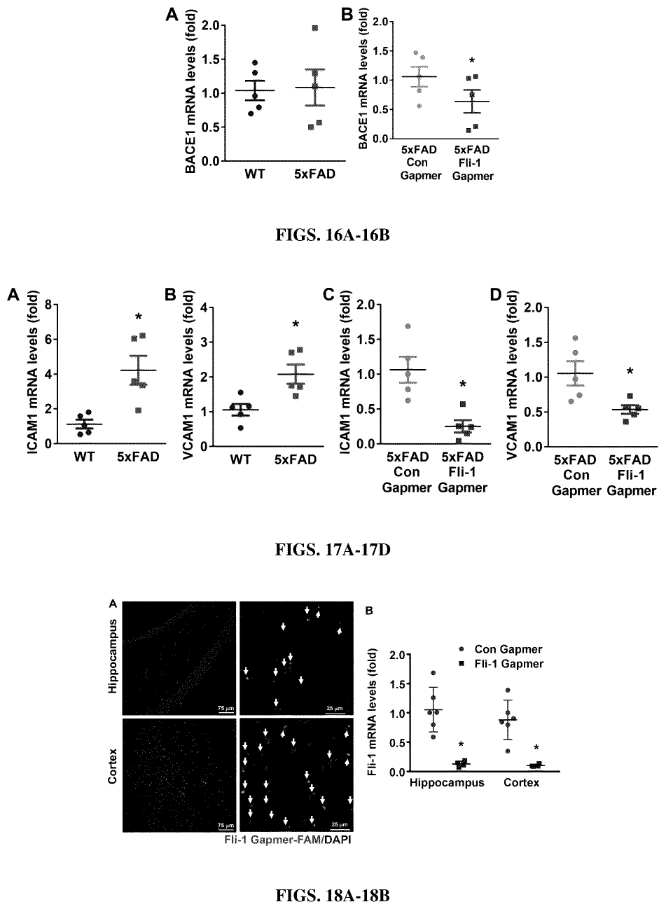

Intrathecal injection of Fli-1 Gapmer decrease Fli-1 levels in the brain. It was also demonstrated that intrathecal injection of green fluorescence FAM-labeled Fli-1 Gapmer entered the brain 24 hours after injection (FIG. 18A), and Fli-1 Gapmers (5 nmol/kg) effectively suppressed Fli-1 levels at 7 days after injection (FIG. 18B).

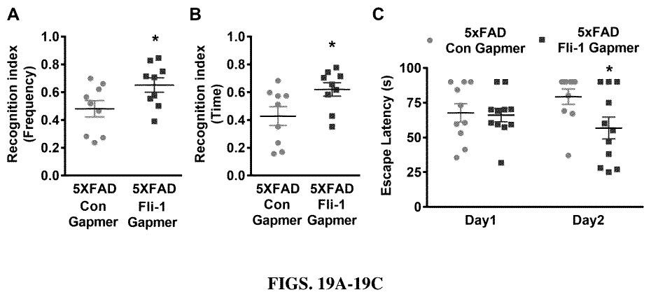

Intrathecal injection of Fli-1 Gapmer ameliorated spatial learning and memory impairment in 5×FAD mice. Fli-1 and control Gapmers (10 nmol/kg) were injected into the intrathecal space of 5×FAD mice at 3, 4, and 5 months of age. 5×FAD mice treated with Fli-1 or control Gapmers were subjected to Novel Object Recognition (NOR) and Morris Water Maze (MWM) tests at 6 months of age. The data demonstrated that 5×FAD mice treated with control Gapmers developed memory impairment similarly to 5×FAD mice, but impairment was significantly reduced in Fli-1 Gapmer treated mice, as evidenced by visiting novel objects more frequently and spending more time with novel objects compared to control Gapmer treated mice (FIG. 19A, B). With the MWM test, we found that after training four times on the first day, 5×FAD mice still used a longer time to find the hidden platform on the second day's test (FIG. 19C). 5×FAD mice treated with Fli-1 Gapmers used significantly less time compared to 5×FAD mice treated with control Gapmers (FIG. 19C). These data demonstrated that intrathecal injection of Fli-1 Gapmer ameliorates spatial learning and memory impairment in 5×FAD mice.

In summary, the present studies identified Fli-1 as a novel regulator of pericyte dysfunction in AD in association with protection against BBB dysfunction, Aβ accumulation and cognitive decline. Thus, it was shown that signaling pathways reducing Fli-1 expression or its downstream mediators represent therapeutic strategies for preventing or mitigating AD-induced cognitive dysfunction. In particular, the data support the concept that the Fli-1 Gapmer represents a novel therapeutic approach for the treatment of Alzheimer's disease

### Example 2—Materials and Methods

Human brain donors. Brain samples were obtained from Carroll A. Campbell, Jr. Neuropathology Laboratory (brain bank) at the Department of Pathology and Laboratory Medicine at the Medical University of South Carolina. These studies were in accordance with the 1964 Helsinki declaration and its later amendments or comparable ethical standards, and approved by the Institutional Review Board at the Medical University of South Carolina. Brain tissues were collected from 21 clinically and neuropathologically confirmed AD patients, 17 cognitively normal individuals (females and males showing neither clinical cognitive impairment/dementia nor brain pathology). The entire human cohort demographics are listed in Table 1. The groups had no significant differences in age, sex, or post-mortem interval (PMI) hours. All samples were previously de-identified.

Animals. The 5×FAD transgenic mice on C57BL/6J background were purchased from the Jackson Laboratory and bred in the animal facility at the Medical University of South Carolina. All animals were allowed free access to food and water and maintained under a facility with 12 h light/dark cycle and constant temperature. All procedures complied with the standards for care and use of animal subjects as stated in the Guide for the Care and Use of Laboratory Animals (Institute of Laboratory Resources, National Academy of Sciences, Bethesda, MD). The protocol for all animal studies was approved by the Institutional Animal Care and Use Committee at the Medical University of South Carolina. The in vivo experimental design can be found in FIG. 14.

Intra-hippocampal injection of control or Fli-1 Gapmer. Control or Fli-1 antisense oligonucleotide Gapmers (Qiagen, Germantown, MD, Germantown, MD) were injected into the hippocampus of 5×FAD transgenic mice as previously described (Butler et al., 2019). Each mouse received two injections at 3 and 4.5 months of age (FIG. 14). Briefly, mice were anesthetized with vaporized isoflurane and fixed in a stereotaxic apparatus. Then, a 2 μl volume of control or Fli-1 Gapmers (25 μM) were injected bilaterally into the hippocampus [anterior-posterior (AP), −2.1 mm: medial-lateral (ML), ±1.6 mm; dorsal-ventral (DV), −1.4 mm] using a Hamilton 5 μl syringe and a 27-gauge needle at 0.2 μl/min for a 10 min duration.

Novel object recognition (NOR). NOR was performed as described previously (Watson et al., 2020) (Stover et al., 2015) in 6-month-old mice. The NOR test Plexiglas box was located in an isolated and illuminated animal testing room. Mice were first given a 10 minute habituation trial with no objects in the test box, and the NOR test was performed 24 h later. In the first trial of NOR, two identical objects were placed in diagonally opposite corners of the test box and the mice were allowed to explore them for 6 minutes. 30 minutes later one of the objects was replaced with a novel object and the mice were again placed in the test box for a second 6 minute trial. Mouse behavior/activity and time spent near each object was recorded by live video tracking and EthoVision XT 13 software (Noldus Information Technology, Leesburg, VA). Both the objects and the box were cleaned with alcohol and dried after each trial to remove olfactory cues. Recognition index (RI) of time was calculated based on the total time (second trial) spent exploring novel object (T_{N}) and familiar (T_{F}) object: RIₜᵢₘₑ=T_{N}/(T_{N}+T_{F}); Recognition index of frequency was calculated based on the number of visits (second trial) to the novel object (F_{N}) and familiar (F_{F}) object: RI_{Frequency}=F_{N}/(F_{N}+F_{F}).

Morris water maze (MWM). MWM test was performed as described previously (Min et al., 2017) in 6-month-old mice. The MWM consists of a raised tank filled halfway with water, which was warmed to a temperature of 25° C. and made opaque with the addition of non-toxic, white tempera paint. A plexiglass escape platform on a raised base was placed in one of four quadrants of the maze, to be slightly submerged (1 cm) under water. Visual cues were placed on the surrounding walls of the maze (N, S, E, W) and remained in place for each day of testing. In brief, each mouse was trained 4 times at 2-minute intervals on day 1. In each trial, mice were placed in water at randomly selected 1 of 4 starting positions and given 90 s to find the platform; the elapsed time for finding the platform was defined as the escape latency for each trial. Latency to reach the location of the platform was monitored and recorded using a high-speed camera and EthoVision XT v13 software (Noldus Information Technology, Leesburg, VA). If the mouse did not locate the platform within 90 s, the mouse was guided gently by hand to the platform and allowed to rest there for 10 s; the escape latency was recorded as 90 s. Following the trial, each mouse was placed in a dry container and allowed to rest for 2 minutes. At day 2, one trial was performed for each mouse to test the spatial learning and memory ability, and the mice were allowed to swim for 90 s at the starting position opposite to the platform and the escape latency was recorded. The test was performed blindly. Mean swim latency for all the trials on each day in each group was calculated.

Human brain pericyte culture and stimulation. Primary human brain pericytes were purchased from ScienCell Research Laboratories and cultured in human pericyte medium (#1201, ScienCell Research Laboratories, Carlsbad, CA). Pericytes were transfected with control or Fli-1 antisense Gapmers (Qiagen, Germantown, MD) for 48 h and cultured with or without freshly aggregated Aβ40 (10 μM, rPeptide, Watkinsville, GA) for 5 consecutive days. Total RNA was collected for further analysis. In another set of experiments, pericytes were transfected with control or Fli-1 antisense Gapmers (Qiagen, Germantown, MD) for 48 h and cultured with or without freshly aggregated Aβ40 (10 μM, rPeptide, Watkinsville, GA) for 7 consecutive days. Immunostaining and cell viability assay were performed. Fresh medium with or without 10 μM Aβ40 was replaced every 2 days until the end of the experiment.

Cell Viability. Cell viability was detected by PrestoBlue™ Cell Viability Reagent (Invitrogen, Waltham, MA) according to the instructions.

Immunocytochemistry. After deparaffinization, postmortem paraffin-embedded human hippocampus sections (4 μm) were treated with antigen retrieval solution at 98° C. for 20 minutes and washed in PBS. Frozen mouse brain tissues were cut into 8-μm sections, fixed with 4% PFA for 15 minutes at room temperature and then washed with PBS. The sections from human or mouse were then incubated in blocking buffer followed by primary antibody overnight in 4° C. with the following combinations: Fli-1 (1:400; Proteintech, Rosemont, IL), CD13 (1:2000; Cell Signaling, Danvers, MA), Aβ (82E1, 1:2000; IBL America), Fli-1 (1:400; Proteintech, Rosemont, IL)/CD13 (1:2000; Cell Signaling, Danvers, MA), cleaved caspase-3 (1:400; Cell Signaling, Danvers, MA)/CD13 (1:2000; Cell Signaling, Danvers, MA). Sections were washed three times by PBS and incubated with Alexa Fluor 488 goat anti-rabbit IgG (H+L) secondary antibody (Invitrogen, Waltham, MA) or Alexa Fluor 594 goat anti-mouse IgG (H+L) secondary antibody (Invitrogen, Waltham, MA), diluted 1:200 in PBS for 1 h at room temperature. For thioflavin-S staining, brain sections were incubated with 0.2% thioflavin-S (T1892, Sigma-Aldrich, St. Louis, MO) diluted in PBS for 10 minutes. After washing with PBS, images were acquired using a keyence BZ-X800 microscope and processed using the keyence software package.

Human brain pericytes grown on 12-well plates coated with 0.2% gelatin were fixed with 4% PFA for 15 minutes at room temperature and then washed with DPBS. After blocking with 1% BSA for 1 h at room temperature, cells were incubated overnight at 4° C. with cleaved caspase-3 antibody (1:400; Cell Signaling, Danvers, MA). Cells were washed with DPBS and incubated for 1 h at room temperature with Alexa Fluor 594 goat anti-rabbit IgG (H+L) secondary antibody (Invitrogen, Waltham, MA), diluted 1:200 in DPBS. After washing with DPBS, images were acquired using a keyence BZ-X800 microscope and processed using the keyence software package.

TUNEL staining. TUNEL staining was performed using a TUNEL kit according to the manufacturer's instructions (Roche, Indianapolis, IN).

Immunostaining quantification. For each biomarker, the fluorescence of specific signals was captured using the same setting and exposure time. For FIG. 1 of postmortem paraffin-embedded human hippocampus sections, quantification was performed from 10 AD donors and 10 age- and sex-matched cognitive normal controls. For FIG. 1B, 4-6 images at 20× objective were taken randomly from each subject. The fluorescence intensity of acquired images was analyzed in the NIH ImageJ software, averaging the value per subject. For FIG. 1C, 4-6 random fields at 20× objective per subject were analyzed for Fli-1⁺ pericyte number, averaging the number per mm² per subject. For FIG. 1D, 3 images at 20× objective were taken randomly from each subject and CD13⁺ pericyte number in each acquired image was analyzed, averaging the number per mm² per subject. For FIG. 1E, 5-6 random fields at 20× objective per subject were analyzed for active caspase-3⁺ pericyte number, averaging the number per mm² per subject. For FIG. 2 of frozen brain tissues, quantification was performed from WT and 5×FAD mice (3 mice/per group). For FIG. 2C, 6 images at 20× objective were taken randomly from the hippocampus area per mouse and the fluorescence intensity in each acquired image was analyzed in the NIH ImageJ software. For FIGS. 2F-G, 6 random images in the hippocampus area per mouse at 20× objective were taken, Fli-1⁺ pericyte number and CD13⁺ pericyte number in each acquired image was analyzed. For FIG. 4 of frozen brain tissues, quantification was performed from WT mice, and 5×FAD mice treated with control or Fli-1 Gapmers (3 mice/per group). For FIGS. 4C-D, 6 random images in the hippocampus area per mouse at 20× objective were taken and Fli-1⁺ pericyte number and CD13⁺ pericyte number in each acquired image was analyzed. For FIG. 4F, 6 random images in the hippocampus area per mouse at 20× objective were taken and the fluorescence intensity of acquired images was analyzed in the NIH ImageJ software. For FIG. 5 of frozen brain tissues, quantification was performed from 5×FAD mice treated with control or Fli-1 Gapmers (3 mice/per group). For 0 FIGS. 5C-D, 6-9 images at 20× objective were taken randomly from the hippocampus area per mouse. The plaque number in each image was counted, and the plaque area in each image was analyzed in the NIH ImageJ software. For FIG. 5G, 6-7 images at 20× objective were taken randomly from the hippocampus area per mouse. The Aβ area in each image was analyzed in the NIH ImageJ software. For FIG. 6 of human brain pericyte immunostaining, quantification was performed from three independent experiments. For FIGS. 6F-G, 10 random images per group at 10× objective were taken and the fluorescence intensity in each acquired image was analyzed in the NIH ImageJ software.

ELISA. The concentration of soluble human Aβ40 and Aβ42 in the hippocampus or cortex from WT mice, and 5×FAD mice treated with con or Fli-1 Gapmers at age of 6.5 months of age was detected. In brief, mice were anesthetized, and hippocampal and cortex tissues were removed and homogenized in RIPA buffer (Cell Signaling, Danvers, MA) on ice for 30 minutes. Samples were then centrifuged for 10 minutes at 4° C. at 12,000×g to obtain the supernatant. Protein concentration was determined by BCA assay and equal amount of proteins were used to detect the concentration of soluble Aβ40 or Aβ42 using an Aβ40 or Aβ42 Human ELISA Kit (Invitrogen, Waltham, MA) according to the manufacturer's instructions. Results are presented as pg Aβ40 or Aβ42 per μg total protein.

Real-time reverse transcription-polymerase chain reaction (RT-PCR). Total RNA was extracted from cultured human brain pericytes, human brain species or mouse brain tissues using the RNeasy Plus Mini kit (Qiagen, Germantown, MD). cDNA was synthesized with High Capacity cDNA Reverse Transcription Kit (Applied Biosystems, Waltham, MA). Quantitative real-time PCR was performed using the SYBR Green PCR Kit (Qiagen, Germantown, MD) and CFX96 Real-Time PCR system (Bio-Rad, Hercules, CA). Primers used in this study were purchased from Qiagen (Germantown, MD). Data were analyzed with 2^{-ΔΔCt} value calculation using GAPDH for normalization.

Western blot analysis. Mouse brain tissues were lysed with ice-cold RIPA lysis buffer (Cell Signaling, Danvers, MA). All lysed samples were kept on ice for 30 minutes and centrifuged for 10 minutes at 4° C. at 12,000×g. Cell lysates were subjected to 12% SDS-PAGE and transferred onto a polyvinylidene difluoride membrane. The membranes were blocked with 7% milk in TBST (20 mM Tris, 500 mM NaCl, and 0.1% Tween 20) for 1 h. After washing with TBST twice, membranes were incubated with primary antibody overnight at 4° C. Primary antibodies to β-actin was obtained from Cell Signaling (Danvers, MA). Fli-1 primary antibody was obtained from Proteintech (Rosemont, IL). The membranes were washed twice with TBST and incubated with HRP conjugated secondary antibody in blocking buffer for 1 h. After washing three times with TBST, immunoreactive bands were visualized by incubation with ECL plus detection reagents (GE Healthcare, Chicago, IL). Images were acquired by ChemiDoc™ Touch Imaging System (Bio-Rad, Hercules, CA) and the densitometry of bands was quantified with Image J2 software.

Intrathecal injection. The mice were anesthetized with isoflurane. The site of injection was prepared by shaving 2 cm² fur and clean with betadine solution and 75% alcohol. A 25 μl Hamilton syringe attached to a 30 G 0.5 in needle will be used for the injection. The spinous process of the L6 was located. The needle was carefully inserted between the groove of L5 and L6 vertebrae. Observation for a tail flick as a sign indicates a successful entry of the needle in the intradural space. Once tail flick was observed the needle position was, immediately, but carefully, secured with one hand and the desired volume of substance (5-10 μl) was injected with the other hand slowly. Once injection is performed, the mouse was moved back to the cage to recover from anesthesia.

Data analysis. Statistical significance was determined by analysis of variance (ANOVA) or Student's t-test using GraphPad Prism software. A value of p<0.05 was considered statistically significant.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Akamata et al., “Endothelin receptor blockade ameliorates vascular fragility in endothelial cell-specific Fli-1-knockout mice by increasing Fli-1 DNA binding ability.” Arthritis Rheumatol, 67, 1335-1344. 2015.
Armulik et al., “Pericytes regulate the blood-brain barrier.” Nature, 468, 557-561. 2010.
Brown et al., “Pericytes and Neurovascular Function in the Healthy and Diseased Brain.” Front Cell Neurosci, 13, 282. 2019.
Butler et al., “Long noncoding RNA NEAT1 mediates neuronal histone methylation and age-related memory impairment.” Sci Signal, 12, 588. 2019.
Cai et al., “Microglia, neuroinflammation, and beta-amyloid protein in Alzheimer's disease.” Int J Neurosci, 124, 307-321. 2014.
Elzi et al., “The role of FLI-1-EWS, a fusion gene reciprocal to EWS-FLI-1, in Ewing sarcoma.” Genes Cancer, 6, 452-461. 2015.
Halliday et al., “Accelerated pericyte degeneration and blood-brain barrier breakdown in apolipoprotein E4 carriers with Alzheimer's disease.” J Cereb Blood Flow Metab, 36, 216-227. 2016.
Ho & Ivashkiv “Downregulation of Friend leukemia virus integration 1 as a feedback mechanism that restrains lipopolysaccharide induction of matrix metalloproteases and interleukin-10 in human macrophages.” J Interferon Cytokine Res, 30, 893-900. 2010.
Iadecola et al., “Vascular Cognitive Impairment and Dementia: JACC Scientific Expert Panel.” J Am Coll Cardiol, 73, 3326-3344. 2019.
Jack et al., “NIA-AA Research Framework: Toward a biological definition of Alzheimer's disease,” Alzheimers Dement, 14, 535-562. 2018.
Jawhar et al., “Motor deficits, neuron loss, and reduced anxiety coinciding with axonal degeneration and intraneuronal Abeta aggregation in the 5×FAD mouse model of Alzheimer's disease.” Neurobiol Aging, 33, 196 e129-140. 2012.
Kisler et al., “Cerebral blood flow regulation and neurovascular dysfunction in Alzheimer disease.” Nat Rev Neurosci, 18, 419-434. 2017.
Kubo et al., “Persistent down-regulation of Fli1, a suppressor of collagen transcription, in fibrotic scleroderma skin.” Am J Pathol, 163, 571-581. 2003.
Li et al., “Fli-1 Governs Pericyte Dysfunction in a Murine Model of Sepsis.” J Infect Dis, 218, 1995-2005. 2018.
Li et al., “Fli-1 transcription factor regulates the expression of caspase-1 in lung pericytes.” Mol Immunol, 108, 1-7. 2019.
Liu et al., “Identification of diterpenoid compounds that interfere with Fli-1 DNA binding to suppress leukemogenesis.” Cell Death Dis, 10, 117. 2019.
Lou et al., “The Fli-1 transcription factor is a critical regulator for controlling the expression of chemokine C—X—C motif ligand 2 (CXCL2).” Mol Immunol, 81, 59-66. 2017.
Ma et al., “Blood-brain barrier-associated pericytes internalize and clear aggregated amyloid-beta42 by LRP1-dependent apolipoprotein E isoform-specific mechanism.” Mol Neurodegener, 13, 57. 2018.
Maiti et al., “Preservation of dendritic spine morphology and postsynaptic signaling markers after treatment with solid lipid curcumin particles in the 5×FAD mouse model of Alzheimer's amyloidosis.” Alzheimers Res Ther, 13, 37. 2021.
Medrano-Jimenez et al., “Malva parviflora extract ameliorates the deleterious effects of a high fat diet on the cognitive deficit in a mouse model of Alzheimer's disease by restoring microglial function via a PPAR-gamma-dependent mechanism.” J Neuroinflammation, 16, 143. 2019.
Min et al., “Administration of bovine casein-derived peptide prevents cognitive decline in Alzheimer disease model mice.” PLoS One, 12, e0171515. 2017.
Parikh et al., “Update on Lupus Nephritis: Core Curriculum 2020.” Am J Kidney Dis, 76, 265-281. 2020.
Pillai et al., “Key inflammatory pathway activations in the MCI stage of Alzheimer's disease.” Ann Clin Transl Neurol, 6, 1248-1262. 2019.
Sagare et al., “Pericyte loss influences Alzheimer-like neurodegeneration in mice.” Nat Commun, 4, 2932. 2013.
Sato et al., “A critical role of the transcription factor fli-1 in murine lupus development by regulation of interleukin-6 expression.” Arthritis Rheumatol, 66, 3436-3444. 2014.
Sengillo et al., “Deficiency in mural vascular cells coincides with blood-brain barrier disruption in Alzheimer's disease.” Brain Pathol, 23, 303-310. 2013.
Shi et al., “Retinal capillary degeneration and blood-retinal barrier disruption in murine models of Alzheimer's disease.” Acta Neuropathol Commun, 8, 202. 2020a.
Shi et al., “Identification of early pericyte loss and vascular amyloidosis in Alzheimer's disease retina.” Acta Neuropathol, 139, 813-836. 2020b.
Sohn et al., “EWS/FLI1 oncogene activates caspase 3 transcription and triggers apoptosis in vivo.” Cancer Res, 70, 1154-1163. 2010.
Stover et al., “Early detection of cognitive deficits in the 3×Tg-AD mouse model of Alzheimer's disease.” Behav Brain Res, 289, 29-38. 2015.
Suie et al., “Nanogel-Facilitated Protein Intracellular Specific Degradation through Trim-Away.” Adv. Funct. Mater., 31, 2021.
Sweeney et al., “Blood-brain barrier breakdown in Alzheimer disease and other neurodegenerative disorders.” Nat Rev Neurol, 14, 133-150. 2018.
Tachibana et al., “Pericyte implantation in the brain enhances cerebral blood flow and reduces amyloid-beta pathology in amyloid model mice.” Exp Neurol, 300, 13-21. 2018.
Takahashi et al., “Epithelial Fli1 deficiency drives systemic autoimmunity and fibrosis: Possible roles in scleroderma.” J Exp Med, 214, 1129-1151. 2017.
Taniguchi et al., “Fibrosis, vascular activation, and immune abnormalities resembling systemic sclerosis in bleomycin-treated Fli-1-haploinsufficient mice.” Arthritis Rheumatol, 67, 517-526. 2015.
Theisen et al., “Therapeutic opportunities in Ewing sarcoma: EWS-FLI inhibition via LSD1 targeting.” Oncotarget, 7, 17616-17630. 2016.
Watson et al., “High-Fat Diet Impairs Tactile Discrimination Memory in the Mouse.” Behav Brain Res, 382, 112454. 2020.
Wu et al., “MicroRNA-181a protects against pericyte apoptosis via directly targeting FOXO1: implication for ameliorated cognitive deficits in APP/PS1 mice.” Aging (Albany NY), 11, 6120-6133. 2019.
Zampeli et al., “A comprehensive evaluation for the treatment of lupus nephritis.” J Autoimmun, 78, 1-10. 2017.
U.S. Patent Publication No. US20160346208A1
U.S. Patent Publication No. US20160145614

1. A method of preventing and/or treating an inflammatory disease in a subject comprising administering an effective amount of friend leukemia virus integration 1 (Fli-1) inhibitor to the subject.

2. The method of claim 1, wherein the Fli-1 inhibitor is a small molecule.

3. (canceled)

4. The method of claim 1, wherein the Fli-1 inhibitor comprises an oligonucleotide.

5. The method of claim 4, wherein the oligonucleotide is double-stranded.

6. (canceled)

7. The method of claim 4, wherein the oligonucleotide comprises at least one modified nucleotide.

8. (canceled)

9. The method of claim 4, wherein the oligonucleotide is a single-stranded oligonucleotide.

10-13. (canceled)

14. The method of claim 4, wherein the oligonucleotide comprises one or more chemically-modified nucleobases.

15-17. (canceled)

18. The method of claim 4, wherein the oligonucleotide comprises one or more locked nucleic acids (LNAs).

19. The method of claim 4, wherein the oligonucleotide comprises DNA and/or RNA nucleobases.

20. (canceled)

21. The method of claim 4, wherein the oligonucleotide comprises a phosphorothioate (PS) backbone modification.

22. The method of claim 1, wherein the single-stranded antisense oligonucleotide is gapmer.

23-47. (canceled)

48. The method of claim 1, wherein the Fli-1 inhibitor is a Fli-1 antibody.

49-56. (canceled)

57. The method of claim 1, wherein the inflammatory disease is Alzheimer's disease, systemic lupus erythematosus, sepsis, or multiple sclerosis.

58. (canceled)

59. The method of claim 1, wherein administering the Fli-1 inhibitor results in decreased pericyte loss, improvement in cognitive deficits, reduced Aβ deposition, and/or decreased BBB breakdown, in decreased BACE1, ICAM1 and/or VCAM1 levels in the hippocampus of the subject as compared to expression prior to administering, in decreased expression of Fli-1 in the brain of the subject as compared to Fli-1 expression prior to administering, or in improved spatial learning and/or memory impairment.

60-62. (canceled)

63. The method of claim 1, wherein the Fli-1 inhibitor is administered more than once.

64. (canceled)

65. The method of claim 1, wherein the Fli-1 inhibitor is administered systemically.

66. (canceled)

67. The method of claim 1, wherein the Fli-1 inhibitor is administered to the brain.

68. (canceled)

69. The method of claim 1, wherein the Fli-1 inhibitor is administered by a liposomal, exosomal, or nanoparticle formulation, or by an extracellular vesicle.

70-76. (canceled)

77. The method of claim 1, further comprising administering a second therapeutic agent to the subject.

78. A composition comprising an antisense gapmer oligonucleotide, wherein the gapmer oligonucleotide is 11-25 nucleotide units in length and able to recruit RNaseH when hybridized to Fli-1.

79-100. (canceled)
